(19) United States
(12) Patent Application Publication
(10) Pub. No.: US 2024/0287105 A1
(43) Pub. Date: Aug. 29, 2024

(54) TREPROSTINIL ANALOGS AND RELATED METHODS OF MAKING AND USING

(71) Applicant: United Therapeutics Corporation, Silver Spring, MD (US)

(72) Inventors: Hitesh Batra, Herndon, VA (US); Sudersan Tuladhar, Silver Spring, MD (US); Sri Harsha Tummala, Owings Mills, MD (US); Yousheng Guan, Fairfax, VA (US); Mark Farquharson, Vienna, VA (US)

(73) Assignee: United Therapeutics Corporation, Silver Spring, MD (US)

(21) Appl. No.: 18/416,389

(22) Filed: Jan. 18, 2024

Related U.S. Application Data
(60) Provisional application No. 63/440,034, filed on Jan. 19, 2023.

(51) Int. Cl. C07F 9/09; A61K 31/222; A61K 31/661; C07C 69/712

(52) U.S. Cl. CPC C07F 9/095; A61K 31/222; A61K 31/661; C07C 69/712

(57) ABSTRACT

Provided are novel treprostinil derivatives, including treprostinil prodrugs and treprostinil analogs, as well as methods of making and using these compounds.

### RELATED APPLICATIONS

The present application claims priority to U.S. Provisional Patent Application No. 63/440,034 filed Jan. 19, 2023, which is incorporated herein by reference in its entirety.

### FIELD

The present disclosure generally relates to prostacyclins and more particularly, to treprostinil, its prodrugs and analogs as well as to related methods of making and using.

### SUMMARY

One embodiment is a compound of formula (1), an enantiomer thereof or a pharmaceutically acceptable salt thereof:

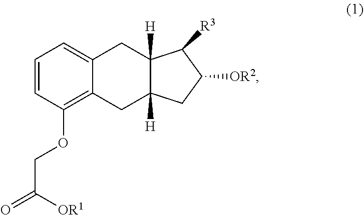

wherein R¹ is H, a C₁-C₃ alkyl group or an acid protecting group, such as a carboxylic acid protecting group; R² is H or an alcohol protecting group; and R³ is

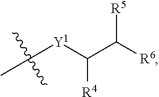

wherein Y¹ is —C≡C—; —CH═CH—; or —(CH₂)ₘ—, m is an integer from 0 to 5; R⁴ is H, OH or ═O; R⁵ is H, OH, a substituted or unsubstituted alkyl group, a substituted or unsubstituted aryl, a substituted or unsubstituted heterocyclic group or a substituted or unsubstituted carbocyclic group; R⁶ is a substituted or unsubstituted alkyl group, a substituted or unsubstituted alkenyl group, a substituted or unsubstituted alkynyl group, a substituted or unsubstituted aryl group, a substituted or unsubstituted cycloalkyl group, wherein for R¹ and R² each being H, R³ is not

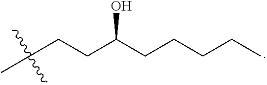 [SMILES: *CC[C@@H](O)CCCCC]

Another embodiment is A compound having formula (2), an enantiomer thereof or a pharmaceutically acceptable salt thereof:

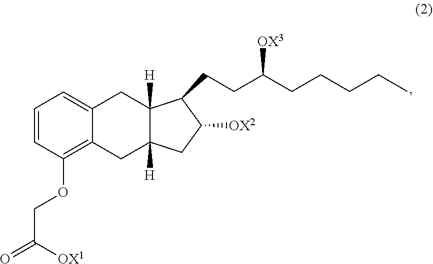

wherein X¹ is hydrogen,

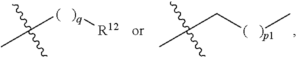

wherein q is 1, 2 or 3, p1 is an integer from 1 to 20, R¹² is a phosphate group or COOH; each of X² and X³ is independently hydrogen,

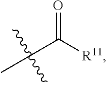

a phosphate, or

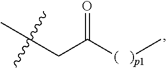

wherein p2 is an integer from 1 to 20, R¹¹ is a C₁-C₈ alkyl group, one or more carbon atoms of said C₁-C₈ alkyl group may be optionally replaced with O, one or more hydrogen atoms of said C₁-C₈ alkyl group may be optionally replaced with halogen; X² is hydrogen, wherein X¹, X² and X³ are not all hydrogen; for X¹ being hydrogen and one of X² and X³ being a phosphate, the other of X² and X³ is not a phosphate or hydrogen; for X¹ being hydrogen and one of X² and X³ being

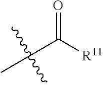

with R¹¹ being an unsubstituted C₁-C₈ alkyl group, the other of X² and X³ is not hydrogen and X² or X³ are not the same.

Yet another embodiments is a compound of formula (3):

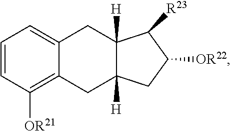

wherein R²¹ is a phenolic protecting group or CH₂COOR²⁴; R²² is an alcohol protecting group; R²³ is a hydroxy terminated alkyl, —C═CH₂, —C≡CH or

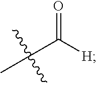

R²⁴ is a carboxylic acid protecting group.

### FIGURES

FIG. 1A-B present Scheme 1, which may be used for synthesizing intermediate(s), which may be further used for synthesizing treprostinil, its prodrugs and analogs.

FIG. 2A-B present Scheme 2, which may be used for synthesizing further intermediate(s) from the intermediate(s) synthesized based on Scheme 1. The further intermediate(s) may be used for synthesizing treprostinil, its prodrugs and analogs.

FIG. 3A-B present Scheme 3, which may be used for synthesizing intermediate(s), which may be further used for synthesizing treprostinil, its prodrugs and analogs.

FIG. 4A-B present Scheme 4, which may be used for synthesizing treprostinil from the intermediate(s) synthesized based on Scheme 3.

FIG. 5A-B present Scheme 5, which may be used for synthesizing a treprostinil analog according to one embodiment from the intermediate(s) synthesized based on Scheme 3.

FIG. 6 shows exemplary treprostinil analogs according to one embodiment.

FIG. 7A-B present Scheme 6, which may be used for synthesizing a treprostinil analog according to one embodiment from the intermediate(s) synthesized based on Scheme 1.

FIG. 8A-B present Scheme 7, which may be used for synthesizing a treprostinil analog according to one embodiment from the intermediate(s) synthesized based on Scheme 1.

FIG. 9A-B present Scheme 8, which may be used for synthesizing a treprostinil analog according to one embodiment from the intermediate(s) synthesized based on Scheme 1.

FIG. 10A-B present Scheme 9, which may be used for synthesizing a treprostinil analog according to one embodiment from the intermediate(s) synthesized based on Scheme 1.

FIG. 11A-B present Scheme 10, which may be used for synthesizing a treprostinil analog according to one embodiment from treprostinil.

FIG. 12A-B present Scheme 11, which may be used for synthesizing a treprostinil analog according to one embodiment from treprostinil.

FIG. 13 shows chemical formulas of treprostinil and selected treprostinil analogs.

FIG. 14A-B show selected treprostinil prodrugs.

FIG. 15 shows exemplary fatty acid conjugates with treprostinil.

### DETAILED DESCRIPTION

As used herein and in the claims, the singular forms “a,” “an,” and “the” include the plural reference unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either.” Thus, unless context indicates otherwise, the word “or” means any one member of a particular list and also includes any combination of members of that list. Other than in the operating examples, or where otherwise indicated, all numbers expressing quantities of ingredients or reaction conditions used herein should be understood as modified in all instances by the term “about.”

Headings are provided for convenience only and are not to be construed to limit the invention in any way. Unless defined otherwise, all technical and scientific terms used herein have the same meaning as those commonly understood to one of ordinary skill in the art. The terminology used herein is for the purpose of describing particular embodiments only, and is not intended to limit the scope of the present invention, which is defined solely by the claims. In order that the present disclosure can be more readily understood, certain terms are first defined. Additional definitions are set forth throughout the detailed description.

All numerical designations, e.g., pH, temperature, time, concentration, and molecular weight, including ranges, are approximations which are varied (+) or (−) by increments of 0.05%, 1%, 2%, 5%, 10% or 20%. It is to be understood, although not always explicitly stated that all numerical designations are preceded by the term “about.” It also is to be understood, although not always explicitly stated, that the reagents described herein are merely exemplary and that equivalents of such are known in the art.

“Pharmaceutically acceptable salt” refers to salts of a compound, which salts are suitable for pharmaceutical use and are derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “Handbook of Pharmaceutically Acceptable Salts,” (2002), Verlag Helvetica Chimica Acta, Zurich, Switzerland), for a discussion of pharmaceutical salts, their selection, preparation, and use.

“Pulmonary hypertension” refers to all forms of pulmonary hypertension, WHO Groups 1-5. Pulmonary arterial hypertension, also referred to as PAH, refers to WHO Group 1 pulmonary hypertension. PAH includes idiopathic, heritable, drug- or toxin-induced, and persistent pulmonary hypertension of the newborn (PPHN).

Generally, pharmaceutically acceptable salts are those salts that retain substantially one or more of the desired pharmacological activities of the parent compound and which are suitable for in vivo administration. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Pharmaceutically acceptable salts include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

Treprostinil, the active ingredient in Remodulin® (treprostinil) Injection, Tyvaso® (treprostinil) Inhalation Solution, and Orenitram® (treprostinil) Extended Release Tablets, was described in U.S. Pat. No. 4,306,075. Methods of making treprostinil and other prostacyclin derivatives are described, for example, in Moriarty, et al., J. Org. Chem. 2004, 69, 1890-1902, Drug of the Future, 2001, 26(4), 364-374, U.S. Pat. Nos. 6,441,245, 6,528,688, 6,700,025, 6,809,223, 6,756,117, 8,461,393, 8,481,782; 8,242,305, 8,497,393, 8,940,930, 9,029,607, 9,156,786 and 9,388,154 9,346,738; U.S. Published Patent Application Nos. 2012-0197041, 2013-0331593, 2014-0024856, 2015-0299091, 2015-0376106, 2016-0107973, 2015-0315114, 2016-0152548, and 2016-0175319; PCT Publication No. WO2016/0055819 and WO2016/081658.

Various uses and/or various forms of treprostinil are disclosed, for examples, in U.S. Pat. Nos. 5,153,222, 5,234,953, 6,521,212, 6,756,033, 6,803,386, 7,199,157, 6,054,486, 7,417,070, 7,384,978, 7,879,909, 8,563,614, 8,252,839, 8,536,363, 8,410,169, 8,232,316, 8,609,728, 8,350,079, 8,349,892, 7,999,007, 8,658,694, 8,653,137, 9,029,607, 8,765,813, 9,050,311, 9,199,908, 9,278,901, 8,747,897, 9,358,240, 9,339,507, 9,255,064, 9,278,902, 9,278,903, 9,758,465; 9,422,223; 9,878,972; 9,624,156; U.S. Published Patent Application Nos. 2009-0036465, 2008-0200449, 2008-0280986, 2009-0124697, 2014-0275616, 2014-0275262, 2013-0184295, 2014-0323567, 2016-0030371, 2016-0051505, 2016-0030355, 2016-0143868, 2015-0328232, 2015-0148414, 2016-0045470, 2016-0129087, 2017-0095432; 2018-0153847; 2021-0121433; 2021-0054009; 2021-0330621; 2021-0378996 and PCT Publications Nos. WO00/57701, WO2016/0105538, WO2016/038532, WO2018/058124, WO2021/041320, WO2022/132655.

Treprostinil has the following chemical formula:

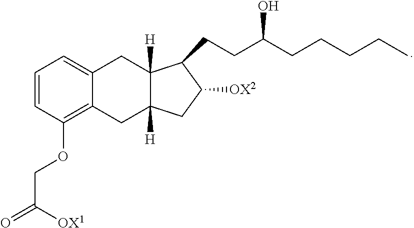

The term “effective amount” may mean an amount of a compound (e.g. a treprostinil analog, a treprostinil prodrug and/or a treprostinil conjugate), which may be necessary to treat the disease or condition. In some embodiments, an effective amount of a treprostinil analog, a treprostinil prodrug and/or a treprostinil conjugate may be the same or similar to an effective amount of treprostinil for treating the same disease or condition. In some embodiments, an effective amount of a treprostinil analog, a treprostinil prodrug and/or a treprostinil conjugate may be different from an effective amount of treprostinil for treating the same disease or condition. A person of ordinary skill in the art would be able to determine and “effective amount” of the treprostinil analog, the treprostinil prodrug and/or the treprostinil conjugate based, for example, on the relevant disease or condition, the amount of treprostinil known to treat, ameliorate, or prevent the disease or condition, and the rate at which the prodrug converts to treprostinil in vivo.

As used herein, Cₘ-Cₙ, such as C₁-C₁₂, C₁-C₈, or C₁-C₆ when used before a group refers to that group containing m to n carbon atoms.

“Optionally substituted” refers to a group selected from that group and a substituted form of that group. Substituents may include any of the groups defined below. In one embodiment, substituents are selected from C₁-C₁₀ or C₁-C₆ alkyl, substituted C₁-C₁₀ or C₁-C₆ alkyl, C₂-C₆ alkenyl, C₂-C₆ alkynyl, C₆-C₁₀ aryl, C₃-C₈ cycloalkyl, C₂-C₁₀ heterocyclyl, C₁-C₁₀ heteroaryl, substituted C₂-C₆ alkenyl, substituted C₂-C₆ alkynyl, substituted C₆-C₁₀ aryl, substituted C₃-C₈ cycloalkyl, substituted C₂-C₁₀ heterocyclyl, substituted C₁-C₁₀ heteroaryl, halo, nitro, cyano, —CO₂H or a C₁-C₆ alkyl ester thereof.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂), and neopentyl ((CH₃)₃CCH₂—).

“Alkenyl” refers to monovalent straight or branched hydrocarbyl groups having from 2 to 10 carbon atoms and preferably 2 to 6 carbon atoms or preferably 2 to 4 carbon atoms and having at least 1 and preferably from 1 to 2 sites of vinyl (>C═C<) unsaturation. Such groups are exemplified, for example, by vinyl, allyl, and but 3-en-1-yl. Included within this term are the cis and trans isomers or mixtures of these isomers.

“Alkynyl” refers to straight or branched monovalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Heteroalkyl” refers to an alkyl group one or more carbons is replaced with —O—, —S—, SO₂, a P containing moiety as provided herein, —NR^{Q}—,

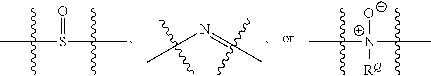

moieties where R^{Q} is H or C₁-C₆ alkyl. Substituted heteroalkyl refers to a heteroalkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxyl, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to a vinyl (unsaturated) carbon atom.

“Heteroalkenyl” refers to an alkenyl group one or more carbons is replaced with —O—, —S—, SO₂, a P containing moiety as provided herein, —NR^{Q}—,

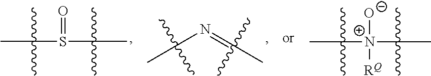

moieties where R^{Q} is H or C₁-C₆ alkyl. Substituted heteroalkenyl refers to a heteroalkenyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Heteroalkynyl” refers to an alkynyl group one or more carbons is replaced with —O—, —S—, SO₂, a P containing moiety as provided herein, —NR^{Q}—,

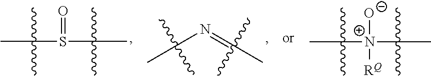

moieties where R^{Q} is H or C₁-C₆ alkyl. Substituted heteroalkynyl refers to a heteroalkynyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Alkylene” refers to divalent saturated aliphatic hydrocarbyl groups having from 1 to 10 carbon atoms, preferably having from 1 to 6 and more preferably 1 to 3 carbon atoms that are either straight chained or branched. This term is exemplified by groups such as methylene (—CH₂—), ethylene (—CH₂CH₂—), n-propylene (—CH₂CH₂CH₂—), iso-propylene (—CH₂CH(CH₃)— or —CH(CH₃)CH₂—), butylene (—CH₂CH₂CH₂CH₂—), isobutylene (—CH₂CH(CH₃—)CH₂—), sec-butylene (—CH₂CH₂(CH₃₋)CH—), and the like. Similarly, “alkenylene” and “alkynylene” refer to an alkylene moiety containing respective 1 or 2 carbon carbon double bonds or a carbon carbon triple bond.

“Substituted alkylene” refers to an alkylene group having from 1 to 3 hydrogens replaced with substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl ester, cycloalkyl, substituted cycloalkyl, heteroaryl, substituted heteroaryl, heterocyclic, substituted heterocyclic, and oxo wherein said substituents are defined herein. In some embodiments, the alkylene has 1 to 2 of the aforementioned groups or having from 1-3 carbon atoms replaced with —O—, —S—, or —NR^{Q}— moieties where R^{Q} is H or C₁-C₆ alkyl. It is to be noted that when the alkylene is substituted by an oxo group, 2 hydrogens attached to the same carbon of the alkylene group are replaced by “═O”. “Substituted alkenylene” and “substituted alkynylene” refer to alkenylene and substituted alkynylene moieties substituted with substituents as described for substituted alkylene.

“Alkynylene” refers to straight or branched divalent hydrocarbyl groups having from 2 to 10 carbon atoms and preferably 2 to 6 carbon atoms or preferably 2 to 3 carbon atoms and having at least 1 and preferably from 1 to 2 sites of acetylenic (—C≡C—) unsaturation. Examples of such alkynylene groups include C≡C— and CH₂C≡C—.

“Substituted alkynylene” refers to alkynylene groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein and with the proviso that any hydroxyl or thiol substitution is not attached to an acetylenic carbon atom.

“Heteroalkylene” refers to an alkylene group wherein one or more carbons is replaced with —O—, —S—, SO₂, a P containing moiety as provided herein, —NR^{Q}—,

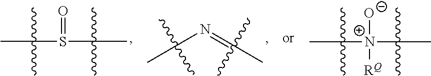

moieties where R^{Q} is H or C₁-C₆ alkyl. “Substituted heteroalkylene” refers to heteroalkynylene groups having from 1 to 3 substituents, and preferably 1 to 2 substituents, selected from the substituents disclosed for substituted alkylene.

“Heteroalkenylene” refers to an alkenylene group wherein one or more carbons is replaced with —O—, —S—, SO₂, a P containing moiety as provided herein, —NR^{Q}—,

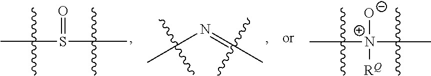

moieties where R^{Q} is H or C₁-C₆ alkyl. “Substituted heteroalkenylene” refers to heteroalkynylene groups having from 1 to 3 substituents, and preferably 1 to 2 substituents, selected from the substituents disclosed for substituted alkenylene.

“Heteroalkynylene” refers to an alkynylene group wherein one or more carbons is replaced with —O—, —S—, SO₂, a P containing moiety as provided herein, —NR^{Q}—,

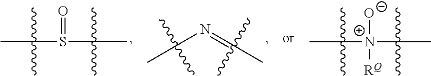

moieties where R^{Q} is H or C₁-C₆ alkyl. “Substituted heteroalkynylene” refers to heteroalkynylene groups having from 1 to 3 substituents, and preferably 1 to 2 substituents, selected from the substituents disclosed for substituted alkynylene.

“Alkoxy” refers to the group 0 alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group 0 (substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR⁴⁷C(O)alkyl, —NR⁴⁷C(O)substituted alkyl, —NR⁴⁷C(O)cycloalkyl, —NR⁴⁷C(O)substituted cycloalkyl, —NR⁴⁷C(O)cycloalkenyl, —NR⁴⁷C(O)substituted cycloalkenyl, —NR⁴⁷C(O)alkenyl, —NR⁴⁷C(O)substituted alkenyl, —NR⁴⁷C(O)alkynyl, —NR⁴⁷C(O)substituted alkynyl, —NR⁴⁷C(O)aryl, —NR⁴⁷C(O)substituted aryl, —NR⁴⁷C(O)heteroaryl, —NR⁴⁷C(O)substituted heteroaryl, —NR⁴⁷C(O)heterocyclic, and NR⁴⁷C(O)substituted heterocyclic wherein R⁴⁷ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl —C(O)O, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group NH₂.

“Substituted amino” refers to the group —NR⁴⁸R⁴⁹ where R⁴⁸ and R⁴⁹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R⁴⁸ and R⁴⁹ are optionally joined, together with the nitrogen bound thereto to form a heterocyclic or substituted heterocyclic group, provided that R⁴⁸ and R⁴⁹ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R⁴⁸ is hydrogen and R⁴⁹ is alkyl, the substituted amino group is sometimes referred to herein as alkylamino. When R⁴⁸ and R⁴⁹ are alkyl, the substituted amino group is sometimes referred to herein as dialkylamino. When referring to a monosubstituted amino, it is meant that either R⁴⁸ or R⁴⁹ is hydrogen but not both. When referring to a disubstituted amino, it is meant that neither R⁴⁸ nor R⁴⁹ are hydrogen.

“Aminocarbonyl” refers to the group —C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR⁴⁷C(O)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic, and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR⁴⁷C(S)NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR⁵⁰R⁵¹ where R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR⁴⁷SO₂NR⁵⁰R⁵¹ where R⁴⁷ is hydrogen or alkyl and R⁵⁰ and R⁵¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR⁵²)NR⁵⁰R⁵¹ where R⁵⁰, R⁵¹, and R⁵² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R⁵⁰ and R⁵¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2 benzoxazolinone, 2H 1,4 benzoxazin 3(4H) one 7 yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Arylene” refers to a divalent aromatic carbocyclic group of from 6 to 14 carbon atoms having a single ring or multiple condensed rings. “Substituted arylene” refers to an arylene having from 1 to 5, preferably 1 to 3, or more preferably 1 to 2 substituents as defined for aryl groups.

“Heteroarylene” refers to a divalent aromatic group of from 1 to 10 carbon atoms and 1 to 4 heteroatoms selected from the group consisting of oxygen, nitrogen and sulfur within the ring. “Substituted heteroarylene” refers to heteroarylene groups that are substituted with from 1 to 5, preferably 1 to 3, or more preferably 1 to 2 substituents selected from the group consisting of the same group of substituents defined for substituted aryl.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group S (substituted aryl), where substituted aryl is as defined herein.

“Carbonyl” refers to the divalent group —C(O)— which is equivalent to —C(═O)—.

“Carboxyl” or “carboxy” refers to COOH or salts thereof.

“Carboxyl ester” or “carboxy ester” refers to the group —C(O)(O)-alkyl, —C(O)(O)-substituted alkyl, —C(O)O-alkenyl, —C(O)(O)-substituted alkenyl, —C(O)(O)-alkynyl, —C(O)(O)-substituted alkynyl, —C(O)(O)-aryl, —C(O)(O)-substituted-aryl, —C(O)(O)-cycloalkyl, —C(O)(O)-substituted cycloalkyl, —C(O)(O)-cycloalkenyl, —C(O)(O)-substituted cycloalkenyl, —C(O)(O)-heteroaryl, —C(O)(O)-substituted heteroaryl, —C(O)(O)-heterocyclic, and —C(O)(O)-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino refers to the group —NR⁴⁷C(O)(O)-alkyl, —NR⁴⁷C(O)(O)-substituted alkyl, —NR⁴⁷C(O)O-alkenyl, —NR⁴⁷C(O)(O)-substituted alkenyl, —NR⁴⁷C(O)(O)-alkynyl, —NR⁴⁷C(O)(O)-substituted alkynyl, —NR⁴⁷C(O)(O)-aryl, —NR⁴⁷C(O)(O)-substituted-aryl, —NR⁴⁷C(O)(O)-cycloalkyl, —NR⁴⁷C(O)(O)-substituted cycloalkyl, —NR⁴⁷C(O)(O)-cycloalkenyl, —NR⁴⁷C(O)(O)-substituted cycloalkenyl, —NR⁴⁷C(O)(O)-heteroaryl, —NR⁴⁷C(O)(O)-substituted heteroaryl, —NR⁴⁷C(O)(O)-heterocyclic, and —NR⁴⁷C(O)(O)-substituted heterocyclic wherein R⁴⁷ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)(O)-substituted alkynyl, —O—C(O)O-aryl, —O—C(O)O-substituted-aryl, —O—C(O)O-cycloalkyl, —O—C(O)O— substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O— substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group CN.

“Cycloalkyl” refers to cyclic alkyl groups of from 3 to 10 carbon atoms having single or multiple cyclic rings including fused, bridged, and spiro ring systems. The fused ring can be an aryl ring provided that the non aryl part is joined to the rest of the molecule. Examples of suitable cycloalkyl groups include, for instance, adamantyl, cyclopropyl, cyclobutyl, cyclopentyl, and cyclooctyl.

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thioxo, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Cyclopropano” refers to:

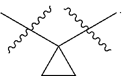 [SMILES: *C1(*)CC1]

“Cyclobutano” refers to

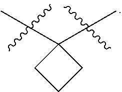 [SMILES: *C1(*)CCC1]

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR⁵³C(═NR⁵³)N(R⁵³)₂ where each R⁵³ is independently selected from the group consisting of hydrogen, alkyl, substituted alkyl, aryl, substituted aryl, heteroaryl, substituted heteroaryl, cycloalkyl, substituted cycloalkyl, heterocyclic, and substituted heterocyclic and two R⁵³ groups attached to a common guanidino nitrogen atom are optionally joined together with the nitrogen bound thereto to form a heterocyclic or substituted heterocyclic group, provided that at least one R⁵³ is not hydrogen, and wherein said substituents are as defined herein.

“Halo” or “halogen” refers to fluoro, chloro, bromo and iodo.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N oxide (N→O), sulfinyl, or sulfonyl moieties. Certain non-limiting examples include pyridinyl, pyrrolyl, indolyl, thiophenyl, oxazolyl, thizolyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more of the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, furan, thiophene, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4 tetrahydroisoquinoline, 4,5,6,7 tetrahydrobenzo[b]thiophene, thiazole, thiazolidine, thiophene, benzo[b]thiophene, morpholinyl, thiomorpholinyl (also referred to as thiamorpholinyl), 1,1 dioxothiomorpholinyl, piperidinyl, pyrrolidine, and tetrahydrofuranyl.

“Nitro” refers to the group —NO₂.

“Oxo” refers to the atom (═O).

Phenylene refers to a divalent aryl ring, where the ring contains 6 carbon atoms.

Substituted phenylene refers to phenylenes which are substituted with 1 to 4, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, substituted sulfonyloxy, thioacyl, thiol, alkylthio, and substituted alkylthio, wherein said substituents are as defined herein.

“Spirocycloalkyl” and “spiro ring systems” refers to divalent cyclic groups from 3 to 10 carbon atoms having a cycloalkyl or heterocycloalkyl ring with a spiro union (the union formed by a single atom which is the only common member of the rings) as exemplified by the following structure:

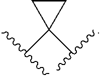 [SMILES: *C1(*)CC1]

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl —SO₂—, phenyl —SO₂—, and 4-methylphenyl-SO₂—.

“Substituted sulfonyloxy” refers to the group —OSO₂-alkyl, —OSO₂-substituted alkyl, —OSO₂— alkenyl, —OSO₂-substituted alkenyl, OSO₂-cycloalkyl, —OSO₂-substituted cycloalkyl, —OSO₂— cycloalkenyl, —OSO₂-substituted cylcoalkenyl, —OSO₂-aryl, —OSO₂-substituted aryl, —OSO₂— heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group SH.

“Thiocarbonyl” refers to the divalent group —C(S)— which is equivalent to —C(═S)—.

“Thioxo” refers to the atom (═S).

“Alkylthio” refers to the group S-alkyl wherein alkyl is as defined herein.

“Substituted alkylthio” refers to the group —S-(substituted alkyl) wherein substituted alkyl is as defined herein.

A substituted ring can be substituted with one or more fused and/or spiro cycles. Such fused cycles include a fused cycloalkyl, a fused heterocyclyl, a fused aryl, a fused heteroaryl ring, each of which rings can be unsubstituted or substituted. Such spiro cycles include a fused cycloalkyl and a fused heterocyclyl, each of which rings can be unsubstituted or substituted.

It is understood that the above definitions are not intended to include impermissible substitution patterns (e.g., methyl substituted with 5 fluoro groups). Such impermissible substitution patterns are well known to the skilled artisan.

The present application discloses a number of novel treprostinil analogs, treprostinil prodrugs and/or treprostinil conjugates. The present application also discloses novel methods for synthesizing treprostinil, its analogs, its prodrugs and/or its conjugates. In addition, the present applications novel intermediate(s), which may be used in those methods.

One embodiment is a compound of formula (1), its enantiomer or its pharmaceutically acceptable salt

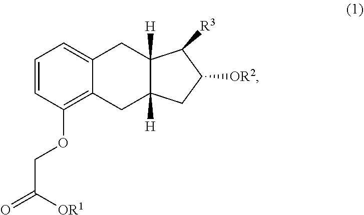

where R¹ may be H, a lower alkyl, such as a C₁-C₃ alkyl group, or an acid protecting group, such as a carboxylic acid protecting group; R² may be H or an alcohol protecting group; and R³ may be

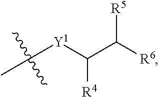

wherein Y¹ is —C≡C—; —CH═CH—; or —(CH₂)ₘ—, m may be an integer from 0 to 5; R⁴ may be H, OH or ═O; R⁵ is H, OH, a substituted or unsubstituted alkyl group, a substituted or unsubstituted aryl, a substituted or unsubstituted heterocyclic group or a substituted or unsubstituted carbocyclic group; R⁶ may be a substituted or unsubstituted alkyl group, a substituted or unsubstituted alkenyl group, a substituted or unsubstituted alkynyl group, a substituted or unsubstituted aryl group, a substituted or unsubstituted cycloalkyl group, where for R¹ and R² each being H, R³ is not

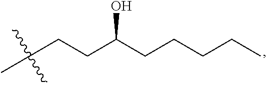 [SMILES: *CC[C@@H](O)CCCCC]

which corresponds to treprostinil.

In some embodiments, R¹ may be H or a lower alkyl, such as C₁-C₃ alkyl, such as methyl, ethyl or propyl. For example, R¹ may be H or methyl.

In some embodiments, R² may be H.

In some embodiments, R³ may be

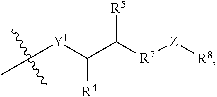

where R⁷ may be, for example, an alkyl group, an alkenyl group or an alkynyl group; Z may be O, CH₂, NH or S; R⁸ may be a heterocyclic group; and R⁴ may be H, OH or ═O.

In some embodiments, R³ may be

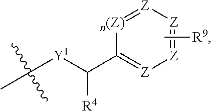

where each Z may be, for example, independently selected from CH, N, O or S, n is 0 or 1, while R⁹ may be an alkyl group, an aryl group, an electron withdrawing group, an electron donating group, a heterocycle or a carbocycle; and R⁴ may be H, OH or ═O.

In some embodiments, R³ is

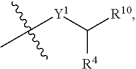

wherein R¹⁰ is a cycloalkyl group having from 3 to 8 carbon atoms, wherein one or more carbon atoms in the cycloalkyl group may be optionally replaced with a heteroatom selected from O, N and S; and R⁴ may be H, OH or ═O.

In some embodiments, R³ may be selected from

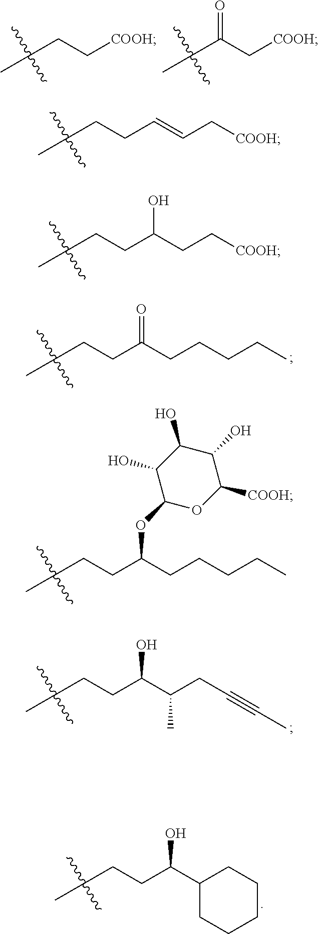

Another embodiment may be a compound having formula (2), its enantiomer or its pharmaceutically acceptable salt:

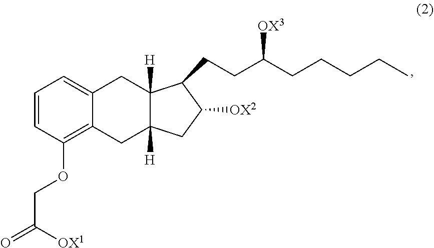

where X¹ may be hydrogen,

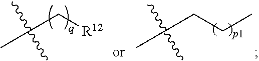

wherein q may be 1, 2 or 3, p1 may be an integer from 1 to 20, R¹² may be a phosphate group or COOH; each of X² and X³ may be independently hydrogen,

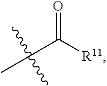

a phosphate, or

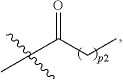

where p2 may be an integer from 1 to 20, R¹¹ is a C₁-C₈ alkyl group, one or more carbon atoms of the C₁-C₈ alkyl group may be optionally replaced with O, one or more hydrogen atoms of the C₁-C₈ alkyl group may be optionally replaced with halogen; X² may be hydrogen, with a proviso that (a) X¹, X² and X³ are not all hydrogen; (b) for X¹ being hydrogen and one of X² and X³ being a phosphate, the other of X² and X³ is not a phosphate or hydrogen; (c) for X¹ being hydrogen and one of X² and X³ being

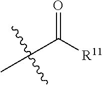

with R¹¹ being an unsubstituted C₁-C₈ alkyl group, the other of X² and X³ is not hydrogen and X² or X³ are not the same.

In some embodiments, X¹ may be

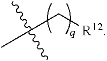

In some embodiments, X² and X³ may be the same group. For example, in some embodiments, each of X² and X³ may be hydrogen. Yet, in some embodiments, each of X² and X³ may be

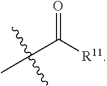

In some embodiments, X¹ may be hydrogen.

In some embodiments, one of X² and X³ is a phosphate and the other of X² and X³ is

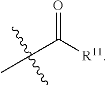

In some embodiments, at least one of X² and X³ is

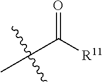

with R¹¹ being a C₁-C₈ alkyl group having one or more carbon atoms of said C₁-C₈ alkyl group replaced with O or having one or more hydrogen atoms replaced with halogen.

In some embodiments, at least one of X² and X³ is

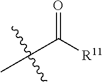

with R¹¹ being a C₁-C₈ alkyl group having one or more hydrogen atoms replaced with halogen. In some embodiments, each of X² and X³ is

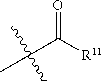

with R¹¹ being a C₁-C₈ alkyl group having one or more hydrogen atoms replaced with halogen.

In some embodiments, X² and X³ are the same.

In some embodiments, at least one of X² and X³ being

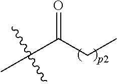

with p2 being an integer from 12 to 16 or X¹ being

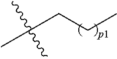

with p1 being an integer from 12 to 16.

In some embodiments, each of X² and X³ is hydrogen and X¹ is

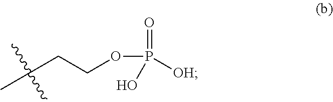 [SMILES: *CCOP(=O)(O)O]

### X₃ is

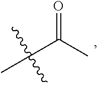 [SMILES: *C(C)=O]

### X₂ is

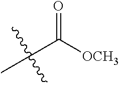 [SMILES: *C(=O)OC]

and X¹ is hydrogen; (c) X³ is

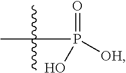 [SMILES: *P(=O)(O)O]

### X² is

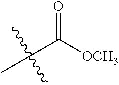 [SMILES: *C(=O)OC]

and X¹ is hydrogen; (d) X³ is

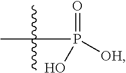 [SMILES: *P(=O)(O)O]

### X² is

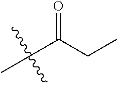 [SMILES: *C(=O)CC]

and X¹ is hydrogen; (e) each of X² and X³ is

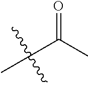 [SMILES: *C(C)=O]

### and X¹ is

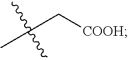 [SMILES: *CC(=O)O]

(f) each of X² and X³ is

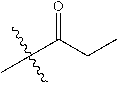 [SMILES: *C(=O)CC]

### and X¹ is

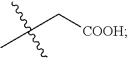 [SMILES: *CC(=O)O]

(g) each X¹ and X² is H and X³ is

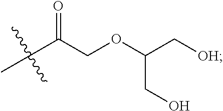 [SMILES: *C(=O)COC(CO)CO]

### (h) X³ is

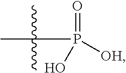 [SMILES: *P(=O)(O)O]

### X² is

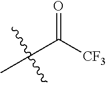 [SMILES: *C(=O)C(F)(F)F]

and X¹ is hydrogen; (i) X² is

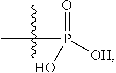 [SMILES: *P(=O)(O)O]

### X³ is

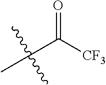 [SMILES: *C(=O)C(F)(F)F]

and X¹ is hydrogen; (j) each of X¹ and X² is hydrogen and X³ is

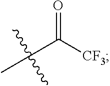 [SMILES: *C(=O)C(F)(F)F]

(k) each of X¹ and X² is hydrogen and X³ is

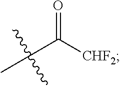 [SMILES: *C(=O)C(F)F]

(l) each of X² and X³ is

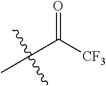 [SMILES: *C(=O)C(F)(F)F]

and X¹ is hydrogen; (m) each of X² and X³ is

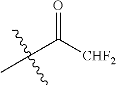 [SMILES: *C(=O)C(F)F]

and X¹ is hydrogen; (n) each of X¹ and X³ is hydrogen and X³ is

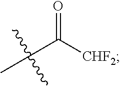 [SMILES: *C(=O)C(F)F]

(o) X¹ is hydrogen; X² is

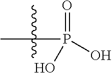 [SMILES: *P(=O)(O)O]

### and X³ is

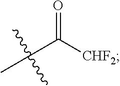 [SMILES: *C(=O)C(F)F]

(p) X¹ is hydrogen; X² is

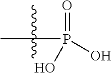 [SMILES: *P(=O)(O)O]

### and X³ is

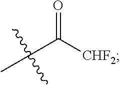 [SMILES: *C(=O)C(F)F]

(q) each of X² and X³ is hydrogen and X¹ is

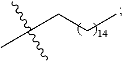

(r) each of X¹ and X² is hydrogen and X³ is

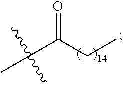

(s) each of X¹ and X³ is hydrogen and X² is

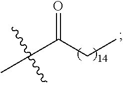

### or (t) X¹ is

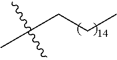

and each X² and X³ is

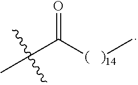

Yet another embodiment is a compound of formula (3):

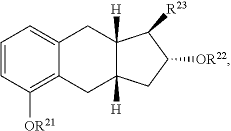

where R²¹ is an phenol protecting group or CH₂COOR²⁴; R²² is an alcohol protecting group; R²³ is a hydroxy terminated alkyl, —C═CH₂, —C≡CH or

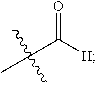

R²⁴ is an alcohol protecting group.

In some embodiments, R²³ is a hydroxy terminated alkyl, such as C₁-C₈ alkyl or C₁-C₄ alkyl terminated by a hydroxy group.

In some embodiments, the compound of formula (3) may be a compound having formula (31):

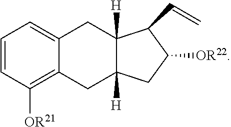

In some embodiments, the compound of formula (3) may be a compound having formula (32):

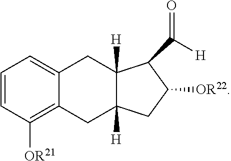

In some embodiments, the compound of formula (3) may be a compound having formula (33):

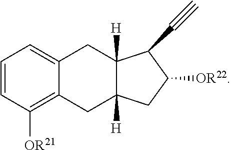

In some embodiments, R²¹ is C₁-C₄ alkyl, a substituted or unsubstituted benzyl or CH₂COOR²⁴, wherein R²⁴ is C₁-C₄ alkyl or a substituted or unsubstituted benzyl.

In some embodiments, R²² is an acetyl group or a silyl containing group.

The compound of formula (3), such the compound of formula (31), (32) or (33) may serve as an intermediate for synthesizing treprostinil analog(s), treprostinil prodrug(s) and/or treprostinil conjugate(s), such as the compound of formula (1) or formula (2) above.

Treprostinil analogs, treprostinil prodrugs and/or treprostinil conjugates may be used for treating any disease or condition that can be treated with treprostinil or its pharmaceutical salts. The treprostinil analogs, treprostinil prodrugs and/or treprostinil conjugates can be formulated into an appropriate pharmaceutical composition depending on the intended application and route of administration (e.g., parenteral, oral, or inhaled). In some embodiments, the disease or condition is one or more selected from the group consisting of pulmonary hypertension, congestive heart failure, peripheral vascular disease, Raynaud's phenomenon, Scleroderma, renal insufficiency, peripheral neuropathy, digital ulcers, intermittent claudication, ischemic limb disease, peripheral ischemic lesions, pulmonary fibrosis and asthma. In some embodiments, the disease is pulmonary hypertension. The pulmonary hypertension can be any form of pulmonary hypertension, e.g., pulmonary arterial hypertension (WHO Group 1 pulmonary hypertension).

Administration may be performed via a route described above, or, for example, orally, intravenously, intra-arterial, intramuscularly, intranasally, rectally, vaginally, or subcutaneously. In some embodiments, the composition is administered by an injection. In some embodiments, the administering is performed orally. In some embodiments, the administering is performed subcutaneously. some embodiments, the administering is performed intravenously.

The subject treated may be a human, canine, feline, aves, non-human primate, bovine, or equine. In some embodiments, the subject is a human.

A compound (e.g. a treprostinil analog, a treprostinil prodrug and/or a treprostinil conjugate) may be provided in a form of a pharmaceutical composition, which may also comprise a pharmaceutically acceptable carrier, excipient, binder, diluent or the like. Such pharmaceutical composition may be manufactured by methods known in the art such as granulating, mixing, dissolving, encapsulating, lyophilizing, emulsifying or levigating processes, among others. The composition may be in the form of, for example, granules, powders, tablets, capsules, syrup, suppositories, injections, emulsions, elixirs, suspensions and solutions. The composition may be formulated for a number of different administration routes, such as, for oral administration, transmucosal administration, rectal administration, transdermal or subcutaneous administration, as well as intrathecal, intravenous, intramuscular, intraperitoneal, intranasal, intraocular or intraventricular injection. The compound (e.g. the treprostinil analog, the treprostinil prodrug and/or the treprostinil conjugate) may be administered by any of the above routes, for example in a local rather than a systemic administration, including as an injection or as a sustained release formulation.

In one embodiment, the pharmaceutical composition can compromise a compound (e.g. a treprostinil analog, a treprostinil prodrug and/or a treprostinil conjugate) and a carrier, such as sterile water. In some embodiments, the compound (e.g. the treprostinil analog, the treprostinil prodrug and/or the treprostinil conjugate) is formulated for subcutaneous administration, and such formulation may or may not include m-cresol or another preservative.

The treprostinil analogs, the treprostinil prodrugs and/or the treprostinil conjugates described herein can be used to treat pulmonary hypertension. In some embodiments, the compound (e.g. the treprostinil analog, the treprostinil prodrug and/or the treprostinil conjugate) can be used to treat pulmonary arterial hypertension (PAH). In some embodiments, the compound (e.g. the treprostinil analog, the treprostinil prodrug and/or the treprostinil conjugate) can be used to treat one or more of WHO Groups 1-5 pulmonary hypertension. Likewise, the treprostinil analogs, the treprostinil prodrugs and/or the treprostinil conjugates described herein can be used to treat any disease or condition for which treprostinil is indicated or useful. The treprostinil analogs, the treprostinil prodrugs and/or the treprostinil conjugates can be administered as the sole therapeutic agent or in addition to other active agents, including treprostinil.

For oral, buccal, and sublingual administration, powders, suspensions, granules, tablets, pills, capsules, gelcaps, and caplets may be acceptable as solid dosage forms. These can be prepared, for example, by mixing one or more compounds, such as treprostinil analogs, treprostinil prodrugs and/or treprostinil conjugates, or pharmaceutically acceptable salts thereof, with at least one additive or excipient such as a starch or other additive. Suitable additives or excipients may be sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms may contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Additionally, dyestuffs or pigments may be added for identification. Tablets may be further treated with suitable coating materials known in the art.

Liquid dosage forms for oral administration may be in the form of pharmaceutically acceptable emulsions, syrups, elixirs, suspensions, slurries and solutions, which may contain an inactive diluent, such as water. Pharmaceutical formulations may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers. Besides those representative dosage forms described above, pharmaceutically acceptable excipients and carriers are generally known to those skilled in the art and can be employed. Such excipients and carriers are described, for example, in “Remingtons Pharmaceutical Sciences” Mack Pub. Co., New Jersey (1991), which is incorporated herein by reference.

A compound (e.g. a treprostinil analog, a treprostinil prodrug and/or a treprostinil conjugate) may be formulated in a formulation suitable for parenteral administration that may comprise sterile aqueous preparations of the compound, or a pharmaceutically acceptable salt thereof, where the preparations may be isotonic with the blood of the intended recipient. These preparations may be administered by means of subcutaneous injection, although administration may also be effected intravenously or by means of intramuscular or intradermal injection. Such preparations may conveniently be prepared by admixing the compound with water or a glycine or citrate buffer and rendering the resulting solution sterile and isotonic with the blood. Injectable formulations may contain from 0.1 to 5% w/v based on weight of treprostinil in the prodrug, the analog and/or the conjugate and may be administered at a rate of 0.1 ml/min/kg. Alternatively, the prodrug, the analog and/or the conjugate may be administered at a rate of 0.625 to 50 ng/kg/min based on weight of treprostinil in the prodrug. Alternatively, the prodrug, the analog and/or the conjugate may be administered at a rate of 10 to 15 ng/kg/min based on weight of treprostinil in the prodrug.

In some embodiments, a concentration of a treprostinil prodrug, a treprostinil analog and/or a treprostinil conjugate in a formulation for parenteral administration, such as intravenous infusion or subcutaneous infusion (including continuous subcutaneous infusion), may be from 0.0005 to 30 mg/mL or from 0.0007 to 50 mg/mL or from 0.001 to 15 mg/mL or any value or subrange within these ranges. Exemplary concentrations may include 0.1 mg/mL, 1 mg/mL, 2.5 mg/mL, 5 mg/mL or 10 mg/mL.

In some embodiments, a formulation of a treprostinil prodrug, a treprostinil analog and/or a treprostinil conjugate for parenteral administration, such as intravenous infusion or subcutaneous infusion (including continuous subcutaneous infusion), may be prepared by admixing the prodrug with a vehicle, such as a buffer. In certain embodiments, the vehicle may be a phosphate containing vehicle, i.e. at least one phosphate salt, which may be for example, dibasic phosphate, such as sodium dibasic phosphate or potassium dibasic phosphate, or tribasic phosphate, such as sodium tribasic phosphate or potassium phosphate. In certain embodiments, the vehicle may also contain a halogen salt, such as a chloride salt, which may be, for example, sodium chloride or potassium chloride. The halogen salt, such as sodium chloride may be used to adjust tonicity of the vehicle. In certain embodiments, it may be preferred that a phosphate and a halogen salt have the same cation. For example, when a phosphate is sodium phosphate, such as sodium tribasic phosphate or sodium tribasic phosphate, a halogen salt may a sodium halogen salt such as sodium chloride. Similarly, when a phosphate is potassium phosphate, such as potassium tribasic phosphate or potassium tribasic phosphate, a halogen salt may a potassium halogen salt such as potassium chloride.

A solvent in the vehicle may contain water. In certain embodiments, water may be the only solvent in the vehicle. Yet in certain embodiments, the vehicle may contain one or more additional solvent in addition to water. In some embodiments, an additional solvent may be a preservative, such as m-cresol.

Preferably, the vehicle is isotonic with blood of a patient, such as a human being. The term isotonic may mean that the osmolarity and ion concentrations of the vehicle match those of the patient, such as human being. Non-limiting example of vehicles include phosphate-buffered saline, which is a water-based salt solution containing disodium hydrogen phosphate, sodium chloride and, in some formulations, potassium chloride and potassium dihydrogen phosphate. Other examples may include a vehicle containing 20 mM disbasic sodium phosphate with 125 mM sodium chloride and a vehicle containing 15 mM sodium phosphate tribasic, 125 mM sodium chloride and 0.3% w/w m-cresol.

### Scheme 1

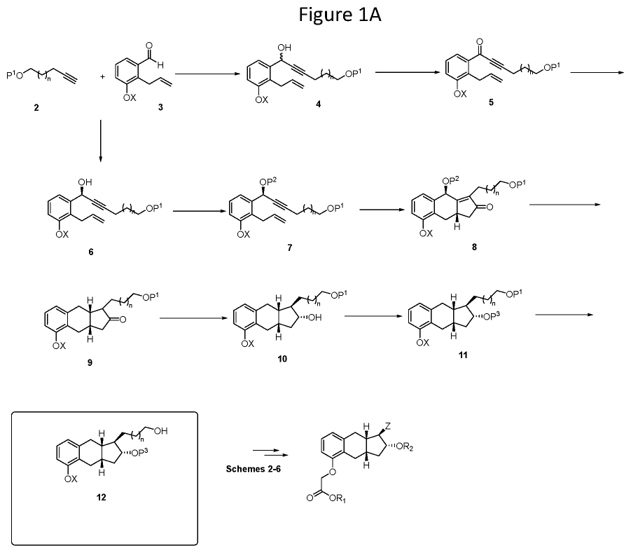

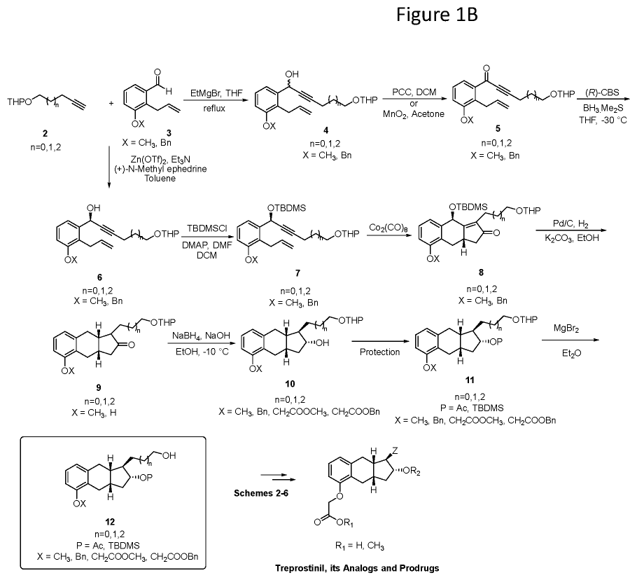

FIG. 1A-B present Scheme 1 which may be used for synthesizing intermediate 12, which may be further used for synthesizing treprostinil, its prodrugs and analogs. FIG. 1A presents a general flow for Scheme 1, while FIG. 1B shows Scheme 1 with exemplary conditions. In Scheme, n is a non-zero integer, such as 0, 1, 2, 3, 4, 5. Each of P¹, P² and P³ is an alcohol (hydroxyl) protecting group. Various protecting groups, including but not limited to, hydroxyl protecting groups and phenol protecting groups are disclosed, for example, in Greene's Protective Groups in Organic Synthesis 5th Edition, Wiley; 5th edition, 2014. Non-limiting examples of hydroxyl protecting groups include 2-tetrahydropyranyl (THP), acetyl (Ac) and silyl ether hydroxyl protecting groups, such as tert-butyldimethylsilyl ether (TBDMS/TBS), trimethylsilyl (TMS); triethylsilyl (TES), tert-butyldiphenylsilyl (TBDPS), triisopropylsilyl (TIPS). X is an phenol protecting group, such as an alkyl, e.g. C₁-C₄ alkyl, or a substituted or unsubstituted benzyl, or CH₂COOR²⁴, where R²⁴ is an alcohol protecting group, such as an alkyl, e.g. C₁-C₄ alkyl, or a substituted or unsubstituted benzyl. The substituted benzyl group in X may be optionally substituted at one or more meta, ortho or para positions with one or more substituents, which may be independently selected from the group consisting of —NO₂, —CN, halogen (e.g., —F, —Cl, —Br or —I), (C₁-C₃)alkyl, halo(C₁-C₃)alkyl, (C₁-C₃)alkoxy and halo(C₁-C₃)alkoxy.

Scheme 1 includes a chiral addition of protected alkyne compound 2 to aldehyde compound 3 to obtain chiral alcohol 6. Alkyne compound 2 may vary in length. In some embodiments, the chiral addition may be performed in a presence of a chiral catalyst, such as one or more of (+)-N-methylephedrine, Zn(OTf)₂/Et₃N or using (1S,2S)-3-(tertiary-butyldimethylsilyloxy)-2-N,N-dimethylamino-L-(para-nitrophenyl)-propane-1-ol. Yet in some embodiments, the chiral addition may be performed through intermediate compounds 4 and 5.

Chiral alcohol 6 may react with an alcohol (hydroxyl) group protecting agent to form protected alcohol compound 7. An “alcohol protecting reagent” is a reagent that converts a —OH group to —OP². In one embodiment, the alcohol protecting reagent is TBDMSCl.

In some embodiments, the reaction of chiral alcohol 6 with an alcohol (hydroxyl) group protecting agent may be carried out in the presence of a base. Suitable base can be used includes, but is not limited to, an alkali carbonate, an alkali hydroxide, an amine and an ammonium hydroxide. In some embodiments, the base may comprise an amine. In some embodiments, the base may comprise dimethylaminopyridine (DMAP).

In some embodiments, the reaction of chiral alcohol 6 with an alcohol (hydroxyl) group protecting agent may be carried out in a suitable solvent or a solvent mixture. For example, the reaction may be carried out in an organic solvent, such as an ethereal solvent (e.g., diethyl ether, methyl tert-butyl ether, tetrahydrofuran, 1,4-dioxane and dimethoxyethane), an aromatic solvent (e.g., benzene and toluene), a chlorinated solvent (e.g., methylene chloride and 1,2-dichloroethane), dimethylformamide, dimethyl sulfoxide, acetonitrile or any mixture of these solvents. In one embodiment, the solvent may include one or more of dimethylformamide (DMF) and dichloromethane (DCM).

Protected alcohol compound 7 may be converted to tricyclic compound 8 through a cyclization reaction. The cyclization reaction may be performed in the presence of a cyclization catalyst, which may be a cobalt-containing cyclization catalyst such as Co₂(CO)₈.

In some embodiments, the cyclization reaction is carried out in an organic solvent or a mixture of organic solvents. Suitable organic solvents include, but are not limited to, ethereal solvents (e.g., diethyl ether, methyl tert-butyl ether, tetrahydrofuran, 1,4-dioxane and dimethoxyethane), aromatic solvents (e.g., benzene and toluene), chlorinated solvents (e.g., methylene chloride and 1,2-dichloroethane), dimethylformamide, dimethyl sulfoxide, acetonitrile or a mixture of any of those solvents. In some embodiments, the cyclization reaction may be carried out in CH₂Cl₂ followed by removal of the solvent by distillation. The reaction may be subsequently carried out in acetonitrile.

Tricyclic compound 8 may be hydrogenated with H₂ to form compound 9. In some embodiments, the hydrogenation reaction is carried out in the presence of a hydrogenation catalyst, such as Pd, C or a combination thereof. In some embodiments, the hydrogenation reaction may be carried in the presence of Pd/C hydrogenation catalyst. In some embodiments, the hydrogenation reaction may carried out in the presence of a base, such as a alkali carbonate (e.g., K₂CO₃). In some embodiments, the hydrogenation reaction is carried out the presence of a hydrogenation catalyst, such as Pd/C, and a base, such as a alkali carbonate (e.g., K₂CO₃).

The hydrogenation reaction may be carried out in an organic solvent, such as ethereal solvents (e.g., diethyl ether, methyl tert-butyl ether, tetrahydrofuran, 1,4-dioxane and dimethoxyethane), aromatic solvents (e.g., benzene and toluene), chlorinated solvents (e.g., methylene chloride and 1,2-dichloroethane), alcohol solvents (e.g., methanol, ethanol, 2-propanol), dimethylformamide, or a combination of any of these solvents. In some embodiments, the hydrogenation reaction is carried out in EtOH.

Compound 9 may be reacted with a reducing agent to form compound 10. A “reducing agent” is a reagent that can convert a carbonyl functional group to an alcohol (hydroxyl) functional group. Suitable reducing agents include, but are not limited to, NaBH₄ and LiAlH₄. In some embodiments, the reaction may be carried out in the presence of a base, such as an alkali hydroxide (e.g. NaOH). The reaction may be carried out in an organic solvent, such as those discussed above for the earlier reactions. In some embodiments, the reaction may be carried out in EtOH.

Compound 10 may be reacted with an alcohol (or hydroxyl) group protecting agent to form compound 11. This reaction protects the cyclopentyl hydroxyl group by replacing hydrogen in the cyclopentyl hydroxyl group in compound 10 with an alcohol (or hydroxyl) group protecting group P³ in compound 11. In some embodiments, P³ may be acetyl (Ac) or silyl ether hydroxyl protecting groups, such as tert-butyldimethylsilyl ether (TBDMS/TBS), trimethylsilyl (TMS); triethylsilyl (TES), tert-tutyldiphenylsilyl (TBDPS), triisopropylsilyl (TIPS). The protecting reaction may be performed in one or more organic solvents discussed above for the earlier reactions.

Compound 11 may be deprotected to form compound 12 by replacing alcohol protecting group P¹ with hydrogen. When P¹ is THP, the deprotecting reaction may be performed in the presence of MgBr₂. The deprotecting reaction may be carried out in an organic solvent, such as those discussed above for the earlier reactions. In some embodiments, the deprotecting reaction may be carried out in diethyl ether.

Compound 12 may be used as an intermediate for synthesizing treprostinil, its analogs, its prodrugs and/or its conjugates.

### Scheme 2

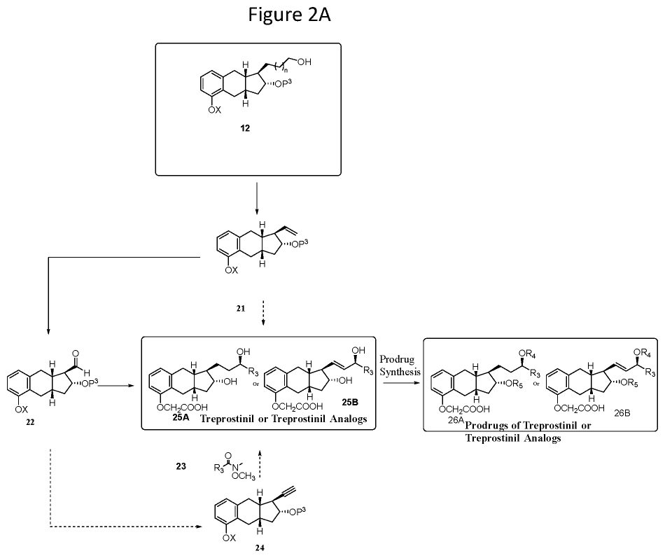

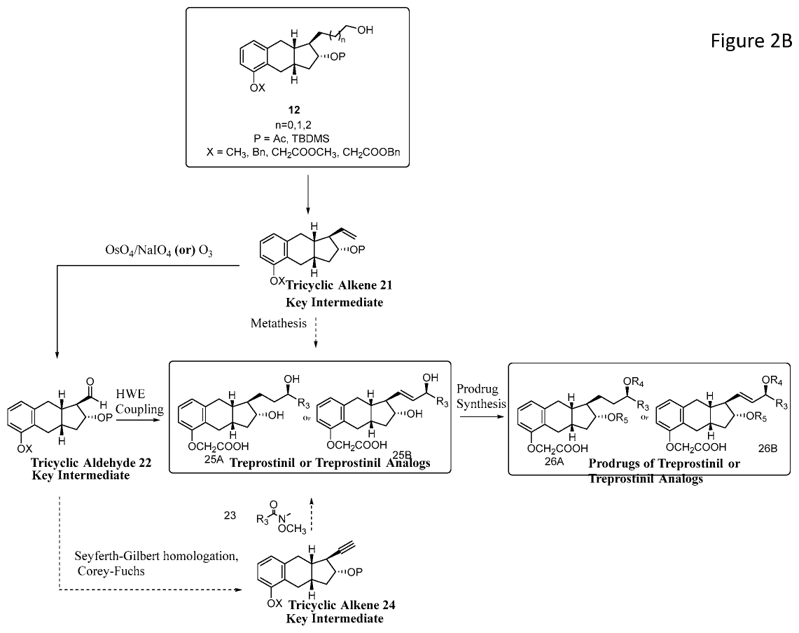

FIG. 2A-B present Scheme 2, which shows how Compound 12 may be used for synthesizing treprostinil, its analogs, its prodrugs and/or its conjugates. FIG. 2A presents a general flow for Scheme 2, while FIG. 2B shows Scheme 2 with exemplary conditions.

Compound 12 may be converted into tricyclic alkene 21, which may also be used as an intermediate for synthesizing treprostinil, its analogs, its prodrugs and/or its conjugates. In some embodiments, tricyclic alkene 21 may be converted directly into treprostinil, treprostinil analog or treprostinil prodrug, such as compounds 25A or 25B via, for example, metathesis.

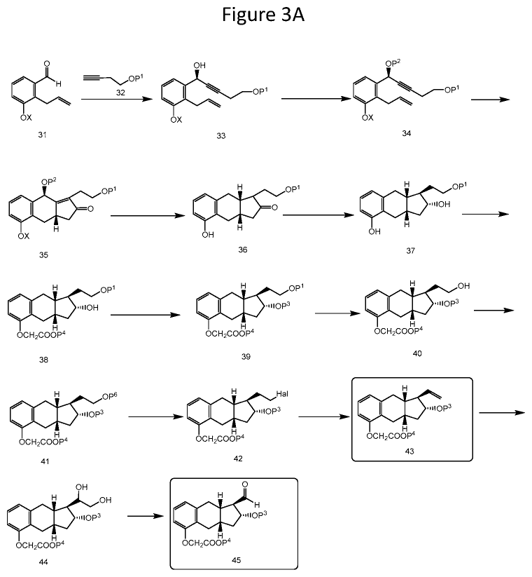

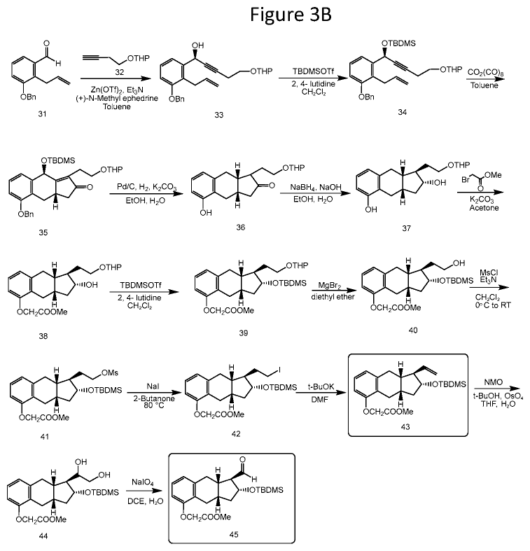

Yet in some other embodiments, tricyclic alkene 21 may be converted into tricyclic aldehyde 22, which may also be used as an intermediate for synthesizing treprostinil, its analogs, its prodrugs and/or its conjugates. Synthesis of tricyclic alkene 21 and tricyclic aldehyde 22 is illustrated in Scheme 3 in FIGS. 3A-B. In some embodiments, tricyclic alkene 21 may be converted into tricyclic aldehyde 22 in the presence of an oxidizer such as OsO₄, NaIO₄, O₃ or any combination of these. For example, the oxidizer may be OsO₄/NaIO₄ or O₃.

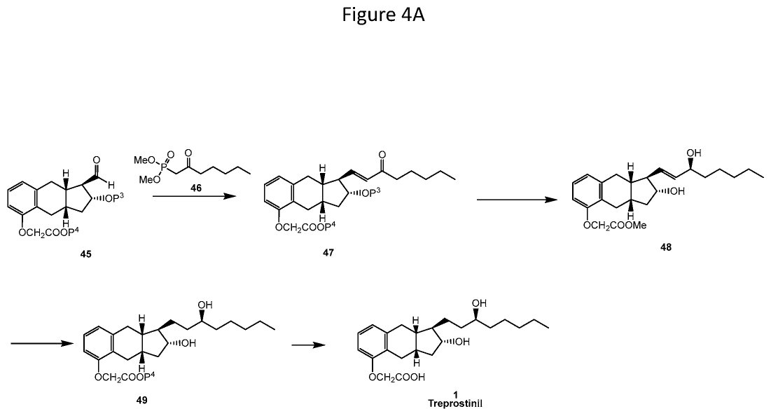

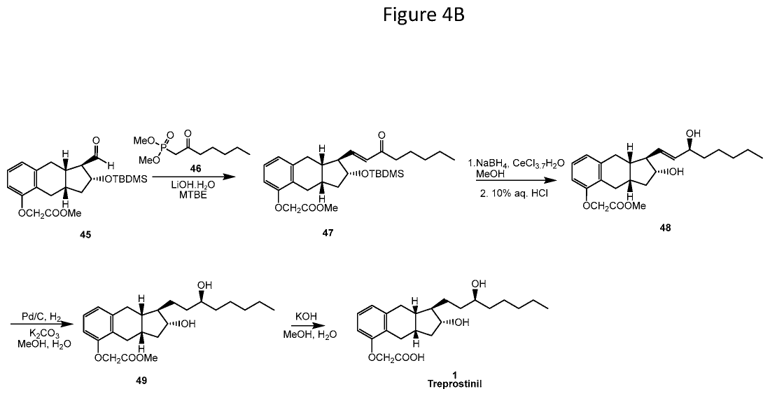

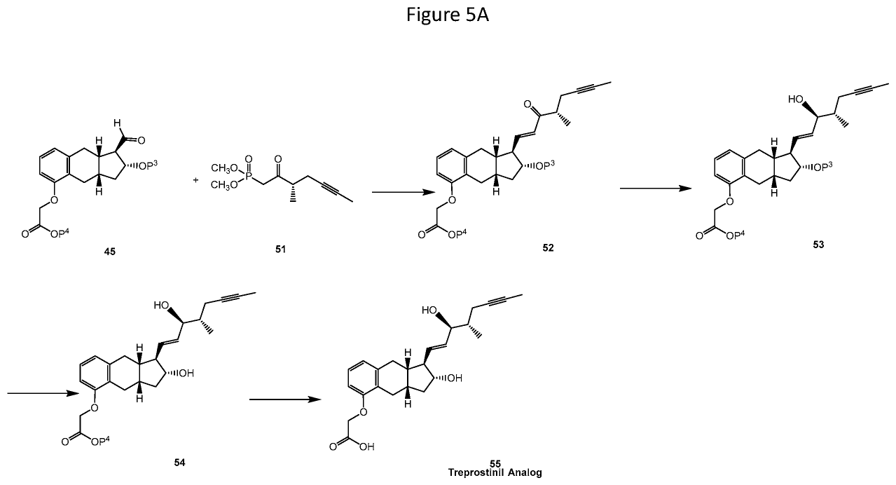

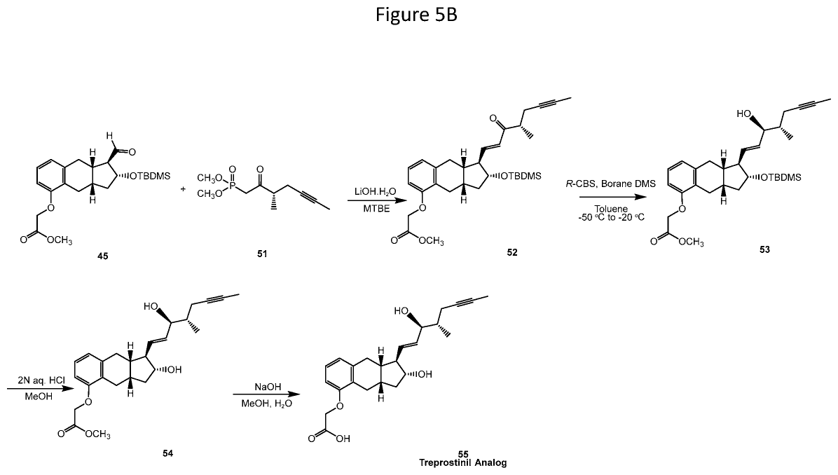

In some embodiments, tricyclic aldehyde 22 may be converted directly into treprostinil, treprostinil analog or treprostinil prodrug, such as compounds 25A or 25B via, as illustrated for example, in Scheme 4 (FIG. 4A-B) or Scheme 5 (FIG. 5A-B). Such conversion may involve a coupling reaction, such as Horner-Wadsworth-Emmons (HWE) coupling reaction.

Yet in some other embodiments, tricyclic aldehyde 22 may be converted into tricyclic alkyne 24, which may also be used as an intermediate for synthesizing treprostinil, its analogs, its prodrugs and/or its conjugates. In some embodiments, tricyclic aldehyde 22 may be converted into tricyclic alkyne 24 via Seyferth-Gilbert homologation reaction and/or Corey-Fuchs reaction. Tricyclic alkyne 24 may be converted directly into treprostinil, treprostinil analog or treprostinil prodrug, such as compounds 25A or 25B, for example, via a reaction with compound 23. Treprostinil, treprostinil analog or treprostinil prodrug, such as compounds 25A or 25B, may be further converted into treprostinil analog or treprostinil prodrug such as compounds 26A or 26B. Various combinations of R³, R⁴, R⁵ (in terms of FIG. 2A-2B) may allow for a large variety of treprostinil analogs and prodrugs as compounds 25A, 25B, 26A and 26B.

### Scheme 3

Scheme 3 in FIG. 3A-B schematically illustrate an exemplary route for synthesizing tricyclic alkene, such as tricyclic alkene 21 in Scheme 2, and tricyclic aldehyde, such as tricyclic aldehyde 22 in Scheme 2. FIG. 3A presents a general flow for Scheme 3, while FIG. 3B shows Scheme 3 with exemplary conditions. Initial reactions in Scheme 3 are similar to those in Scheme 1.

Scheme 3 includes a chiral addiction of protected alkyne compound 32 (compound 2 in Scheme 1 for n=0 to aldehyde compound 31 (Compound 3 in Scheme 1) to obtain chiral alcohol 33 (Compound 6 in Scheme 1 for n=0)

Chiral alcohol 33 may react with an alcohol (hydroxyl) group protecting agent to form protected alcohol compound 34 (Compound 7 in Scheme 1 for n=0).

Protected alcohol compound 34 may be converted to tricyclic compound 35 (Compound 8 in Scheme 1 for n=0) through a cyclization reaction.

Tricyclic compound 35 may be hydrogenated with H₂ to form compound 36, which may be Compound 9 in Scheme 1 for n=0, and X═H when X is Bn in the earlier compounds of that Scheme or Compound 9 in Scheme for n=0 and X being CH₃ when X is CH₃. During the hydrogenolysis, Benzyl group as X will be cleaved and replaced H, only CH₃ will not cleave under the hydrogenolysis conditions.

Compound 36 may be reacted with a reducing agent to form compound 37 (Compound 10 in Scheme 1 for n=0 and X═H.

Compound 37 is converted into compound 38 (Compound 10 in Scheme 1 for n=0 and X ═CH₂COOP⁴ with P⁴ being an carboxylic acid protecting group, such as C₁-C₄ alkyl or a substituted or unsubstituted benzyl).

Compound 38 may be reacted with an alcohol (or hydroxyl) group protecting agent to form compound 39 (Compound 11 in Scheme 1 with n=0 and X═CH₂COOP⁴.

Compound 39 may be deprotected to form compound 40 (Compound 12 in Scheme 1 with n=0 and X═CH₂COOP⁴) by replacing alcohol protecting group P¹ with hydrogen.

Compound 40 may reacted an alcohol (hydroxyl) protecting agent to replace terminal hydrogen with alcohol protecting group P⁶, which may be different from alcohol protecting group P¹, to form compound 41. For example, in some embodiments, P⁶ may be a sulfonated alcohol, such as mesyl or tosyl. The reaction with a sulfonated alcohol, such as mesyl chloride or tosyl chloride, may be performed in one or more solvents discussed above. In some embodiments, the solvent may comprise trimethylamine, dichloromethane or their combination.

Compound 41 may be converted into compound 42 by replacing OP⁶ (with P⁶ being a sulfonated alcohol) with a halogen, such as Br or I. Such conversion may be performed for example, by reacting compound 41 with a halogen salt of an alkali metal, such as Na or K. For example, compound 41 may be reacted with NaI, NaBr, KI or KBr. The reaction may be performed in one or more of the solvents discussed above. In some embodiments, the reaction may be carried out in 2-butanone.

Compound 42 may be converted into tricyclic alkene compound 43 (Compound 21 in Scheme 2 with R¹ being P³ and R² being CH₂COOP⁴). Such conversion may be performed, for example, by reacting compound 42 with a base, such as potassium tert-butoxide. The reaction may be performed in one or more of the solvents discussed above. In some embodiments, the reaction may be carried out in DMF.

Compound 43 may be converted into compound 44. Such conversion may be carried out, for example, by reacting compound 43 with an oxidizing agent, which may comprise, for example, 4-methylmorpholine 4-oxide (NMO), OSO₄ or their combination. The reaction may be performed in one or more of the solvents discussed above. In some embodiments, the reaction may be carried out in tetrahydrofuran (THF), water or their combination.

Compound 44 may be converted into tricyclic aldehyde compound 45 (Compound 22 in Scheme 2 with R¹ being P³ and R² being CH₂COOP⁴. Such conversion may be carried out, for example, by reacting compound 43 with an oxidizing agent, which may comprise, for example, such as OsO₄, NaIO₄, O₃ or a combination of two or more of them. The reaction may be performed in one or more of the solvents discussed above. In some embodiments, the reaction may be carried out in 1,2-dichloroethane (DCE), water or their combination.

Although not shown in FIG. 3A-B, in some embodiments, tricyclic alkene compound 43 may be converted into tricyclic aldehyde compound 45 via ozonolysis.

Tricyclic alkene compound 43 and tricyclic aldehyde compound 45 may be used for synthesizing treprostinil, treprostinil analogs and treprostinil prodrugs.

### Scheme 4

Scheme 4 in FIG. 4A-B schematically illustrate an exemplary route for synthesizing treprostinil from tricyclic aldehyde compound 45, which may be synthesized, for example, according to Scheme 3. FIG. 4A presents a general flow for Scheme 4, while FIG. 4B shows Scheme 4 with exemplary conditions.

Tricyclic aldehyde compound 45 may be converted into compound 47 via a side chain coupling reaction. The side chain coupling reaction may be a phosphonate chain coupling reaction in which tricyclic aldehyde compound may be reacted with a phosphonate compound 46 to form compound 47. The phosphonate chain coupling reaction may be performed in a solvent, such as methyl tert-butyl ether, in the presence of a base, such as lithium hydroxide monohydrate, e.g. LiOH·H₂O.

Compound 47 may be converted into compound 48 in a reduction reaction. The reduction reaction may be a Luche reduction reaction, which may be performed, for example, in the presence of sodium borohydride (NaBH₄) and a lanthanide chloride, such as cerium(III) chloride (CeCl₃), in an alcohol, such as methanol or ethanol.

Compound 48 may be further converted into compound 49 in a reduction reaction. The reduction reaction may be a hydrogenation reaction performed in the present of a hydrogenation catalyst, such as Palladium on carbon (Pd/C).

Compound 49 may be converted into treprostinil 1 via a deprotection reaction.

### Scheme 5

Scheme 5 in FIG. 5A-B schematically illustrate an exemplary route for synthesizing treprostinil analog 55 from tricyclic aldehyde compound 45, which may be synthesized, for example, according to Scheme 3. FIG. 5A presents a general flow for Scheme 5, while FIG. 5B shows Scheme 5 with exemplary conditions.

Tricyclic aldehyde compound 45 may be converted into compound 52 by a side chain coupling reaction. The side chain coupling reaction may be a phosphonate chain coupling reaction in which tricyclic aldehyde compound may be reacted with a phosphonate compound 51 to form compound 52. The phosphonate chain coupling reaction may be performed in a solvent, such as methyl tert-butyl ether, in the presence of a base, such as lithium hydroxide monohydrate, e.g. LiOH·H₂O.

Compound 52 may be converted into compound 53 via a reduction reaction. The reduction reaction may be an enantioselective reduction reaction performed in a presence of a chiral catalyst, such as a chiral oxazaborolidine catalyst, e.g. (R)-2-Methyl-CBS-oxazaborolidine.

Compound 53 may be converted into compound 54 in a cyclopentyl deprotecting reaction.

Compound 54 may be converted into compound 55 via a deprotection reaction, such as a base hydrolysis deprotection reaction.

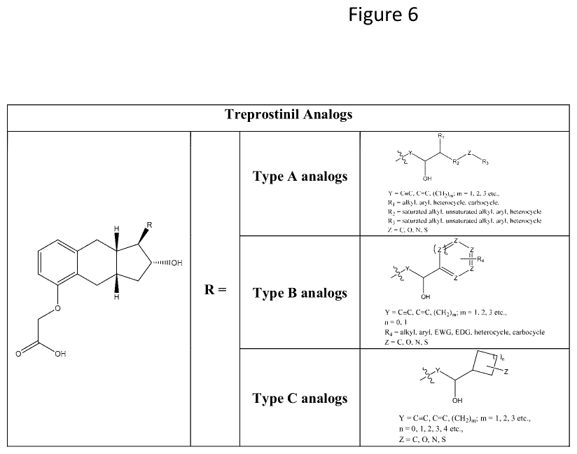

FIG. 6 illustrates treprostinil analogs, which may be produced using the discussed above and below methods.

### Scheme 6

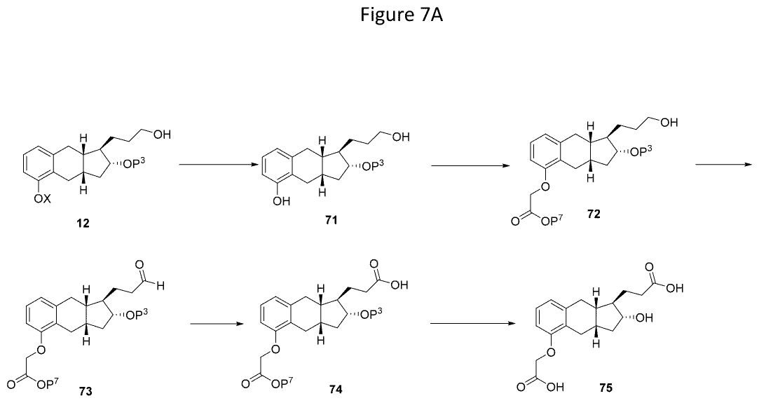

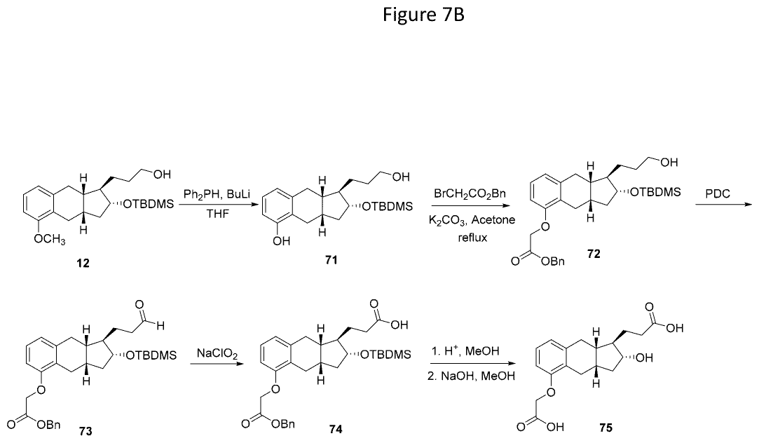

Scheme 6 in FIG. 7A-B schematically illustrates an exemplary route for synthesizing compound 75 from compound 12, which may be synthesized, for example, according to Scheme 1. FIG. 7A presents a general flow for Scheme 6, while FIG. 7B shows Scheme 6 with exemplary conditions.

Compound 12 may be converted into compound 71 via a deprotection reaction, which may be a selective deprotection reaction. The deprotection reaction may be demethylation reaction, which may be performed in the presence of a demethylation condition, such as a combination of Ph₂PH and BuLi or a reaction product of such combination, e.g. Ph₂PLi.

Compound 71 may be converted into compound 72 via a reaction with a protected haloacetate, such as benzylbromoacetate.

Compound 72 may be converted into aldehyde compound 73. This reaction may be performed in a presence of an oxidizing reagent, such as pyridinium dichromate (PDC).

Aldehyde compound 73 may be converted into compound 74 via an oxidation reaction. The oxidation reaction may be the Pinnick oxidation performed in the presence of NaClO₂.

Compound 74 may be converted into treprostinil analog 75 via a deprotection reaction.

### Scheme 7

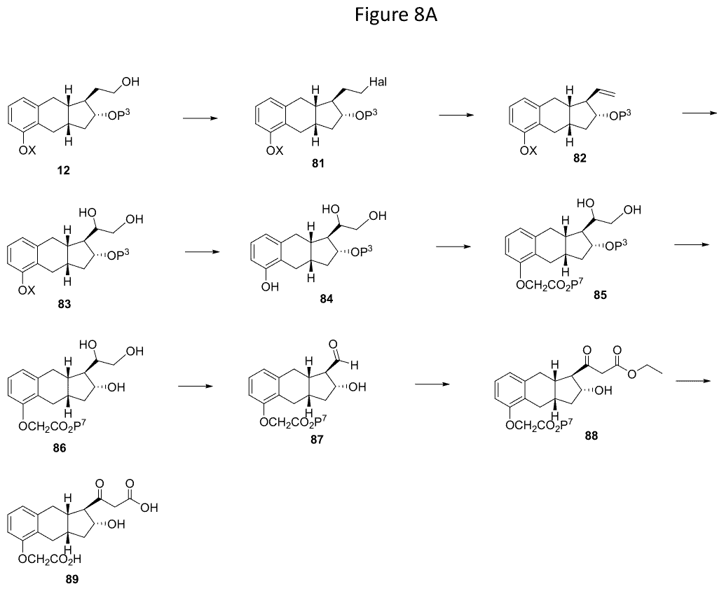

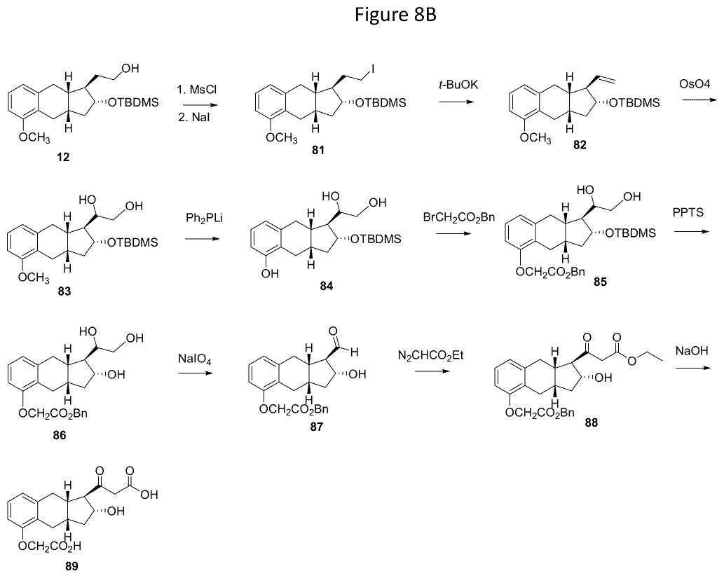

Scheme 7 in FIG. 8A-B schematically illustrates an exemplary route for synthesizing compound 89 from compound 12, which may be synthesized, for example, according to Scheme 1. FIG. 8A presents a general flow for Scheme 7, while FIG. 8B shows Scheme 7 with exemplary conditions.

Compound 12 may be converted into compound 81 by replacing terminal hydroxy group with a halogen, such as Br or I. Such conversion may be performed for example, by reacting compound 12 with mesyl chloride followed by a halogen salt of an alkali metal, such as Na or K. For example, compound 12 may be reacted with mesyl chloride followed by NaI, NaBr, KI or KBr. The reaction may be performed in one or more of the solvents discussed above. In some embodiments, the reaction may be carried out in 2-butanone.

Compound 81 may be converted into tricyclic alkene compound 82. Such conversion may be performed, for example, by reacting compound 81 with a base, such as potassium tert-butoxide. The reaction may be performed in one or more of the solvents discussed above. In some embodiments, the reaction may be carried out in DMF.

Compound 82 may be converted into compound 83 via an oxidation reaction, which may be performed in the presence of an oxidizer such as OsO₄.

Compound 83 may be converted into compound 84 via a deprotection reaction, which may be a selective deprotection reaction. The deprotection reaction may be demethylation reaction, which may be performed in the presence of a demethylation condition such as a combination of Ph₂PH and BuLi or a reaction product of such combination, e.g. Ph₂PLi.

Compound 84 may be converted into compound 85 via a reaction with a protected haloacetate, such as benzylbromoacetate.

Compound 85 may be converted into compound 86 via a deprotection reaction, which may be a selective deprotection at the cyclopentyl ring. Such deprotection may be performed in the presence of a deprotection agent, such as pyridinium p-toluenesulfonate (PPTS).

Compound 86 may be converted into tricyclic aldehyde compound 87 via an oxidation reaction, which may be performed in the presence of an oxidizer such as OsO₄, NaIO₄, O₃ or a combination of two or more of them.

Compound 87 may converted into compound 88 via a reaction with N₂CHCO₂Et.

Compound 88 may be converted into treprostinil analog 89 via a deprotection reaction, which may be performed in a presence of a base.

### Scheme 8

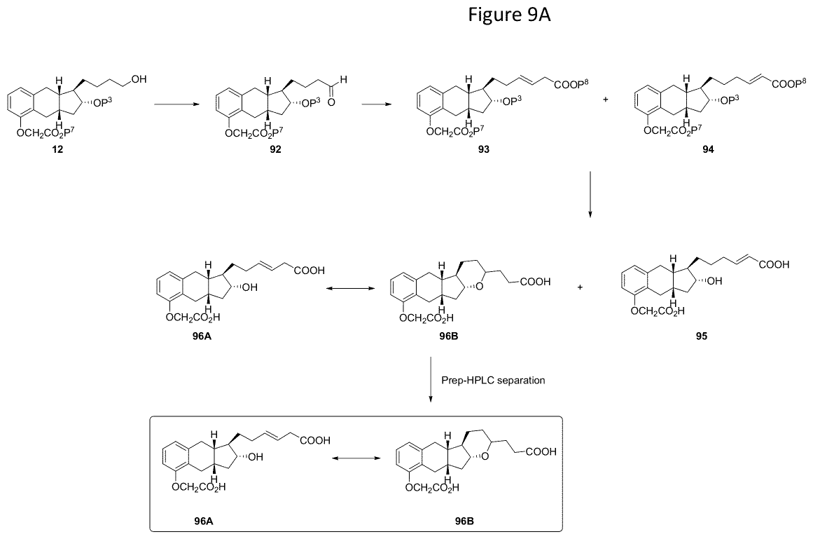

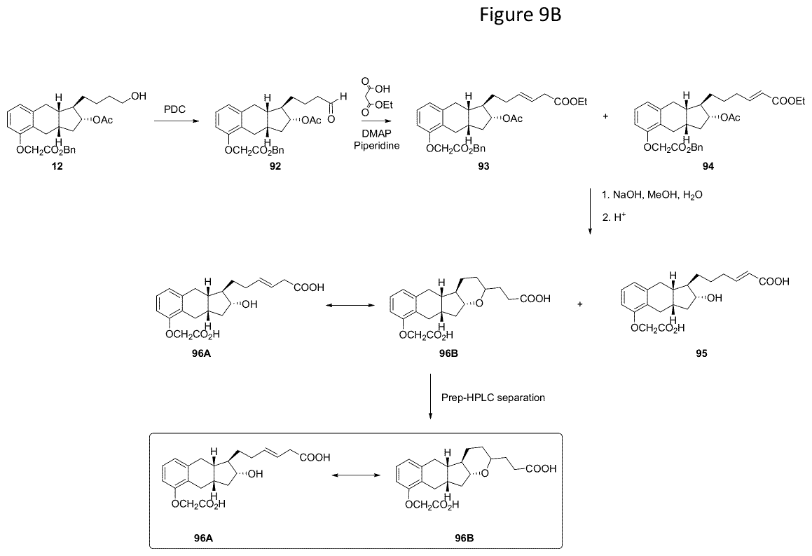

Scheme 8 in FIG. 9A-B schematically illustrates an exemplary route for synthesizing treprostinil analog 96A from compound 12, which may be synthesized, for example, according to Scheme 1. FIG. 9A presents a general flow for Scheme 8, while FIG. 9B shows Scheme 8 with exemplary conditions.

Compound 12 may be converted into aldehyde compound 92. This reaction may be performed in a presence of a oxidizing reagent, such as pyridinium dichromate (PDC).

Aldehyde compound 92 may be converted into a mixture of compound 93 and 94, which may be further converted into a mixture of compounds 96A, 96B and 95 via a deprotection reaction.

Compound 96A may be separated from that mixture using a separation technique, such as HPLC.

### Scheme 9

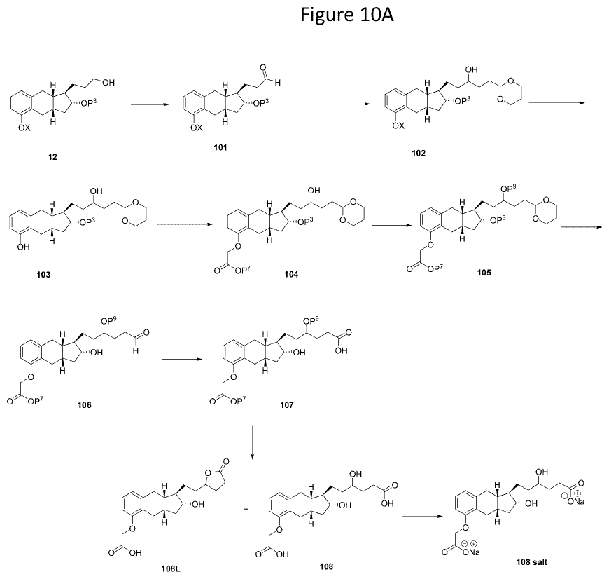

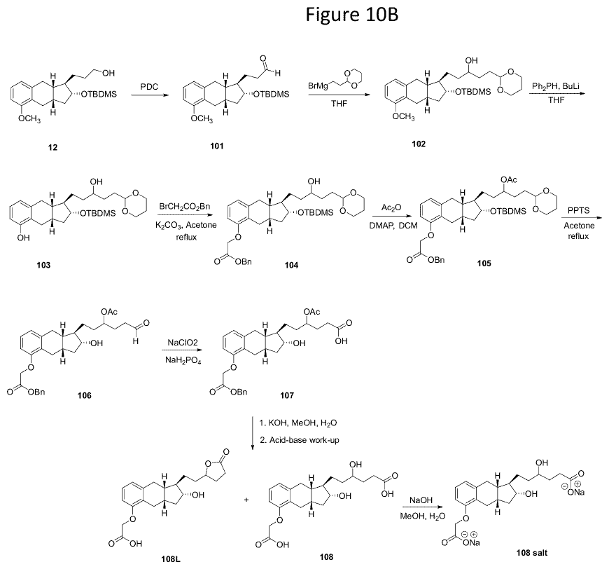

Scheme 9 in FIG. 10A-B schematically illustrates an exemplary route for synthesizing compound 108 from compound 12, which may be synthesized, for example, according to Scheme 1. FIG. 10A presents a general flow for Scheme 9, while FIG. 10B shows Scheme 9 with exemplary conditions.

Compound 12 may be converted into aldehyde compound 101. This reaction may be performed in a presence of an oxidizing reagent, such as pyridinium dichromate (PDC).

Compound 101 may be then converted into compound 102.

Compound 102 may be converted into compound 103 via a deprotection reaction, which may be a selective deprotection reaction. The deprotection reaction may be demethylation reaction, which may be performed in the presence of a demethylation condition such as a combination of Ph₂PH and BuLi or a reaction product of such combination, e.g. Ph₂PLi.

Compound 103 may be converted into compound 104 via a reaction with a protected haloacetate, such as benzyl bromoacetate.

Compound 104 may be converted into compound 105 by protecting the hydroxy group.

Compound 105 may be converted into compound 106 in the presence of a deprotection agent, such as pyridinium p-toluenesulfonate (PPTS).

Compound 106 may be converted into compound 107 via an oxidation reaction. The oxidation reaction may be the Pinnick oxidation performed in the presence of an oxidizer, such as NaClO₂.

Compound 107 may be then converted into a mixture of compound 108L and treprostinil analog 108 via a deprotection reaction. Treprostinil analog 108 may be separated from such mixture using a separation technique, such as an HPLC. Treprostinil analog 108 may converted into its salt, such as a sodium salt.

### Scheme 10

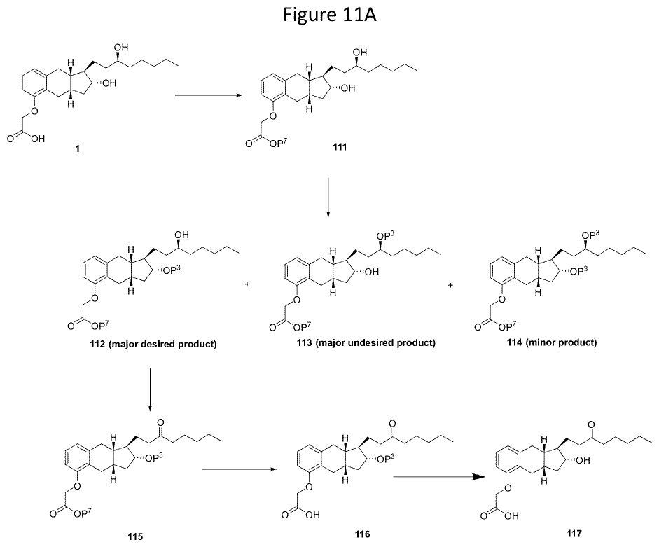

Scheme 10 in FIG. 11A-B schematically illustrates an exemplary route for synthesizing compound 117 from treprostinil. FIG. 11A presents a general flow for Scheme 10, while FIG. 11B shows Scheme 10 with exemplary conditions.

Treprostinil 1 may be converted into compound 111 by selective protecting carboxylic acid group.

Compound 111 may be reacted with a silyl protecting agent such as tert-butyldimethylsilyl chloride TBDMSCl or tert-butyldiphenylsilyl chloride TBDPSCl. Such reaction may lead to a mixture of desired compound 112 with the silyl protecting group being on the cyclopentyl's hydroxy group but not the alkyl chain's hydroxy group; undesired compound 113 with the silyl protecting group being on the alkyl chain's hydroxy group but not on the cyclopentyl's hydroxy group and minor product 114 with the silyl protecting group being on both the alkyl chain's hydroxy group and the cyclopentyl's hydroxy group.

Desired compound 112 may be separated from the mixture using a separation technique, such as column chromatography, an HPLC and SFC etc.

Compound 112 may be converted into compound 115. Such reaction may be performed in the presence of an oxidizing agent such as pyridinium chlorochromate (PCC).

Compound 115 may be converted into compound 116 by deprotecting the benzyl ester of carboxylic acid's group via, for example, hydrogenolysis.

Compound 116 may be converted into treprostinil analog 117 via a deprotection reaction.

### Scheme 11

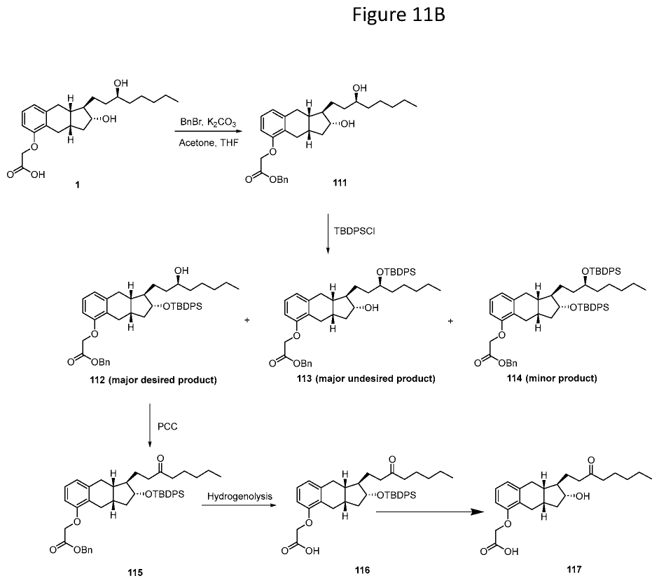

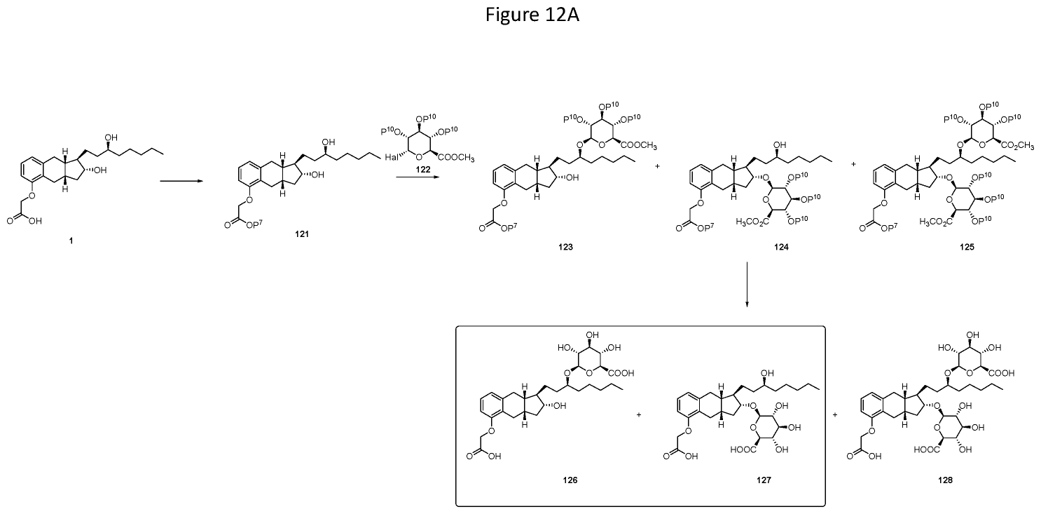

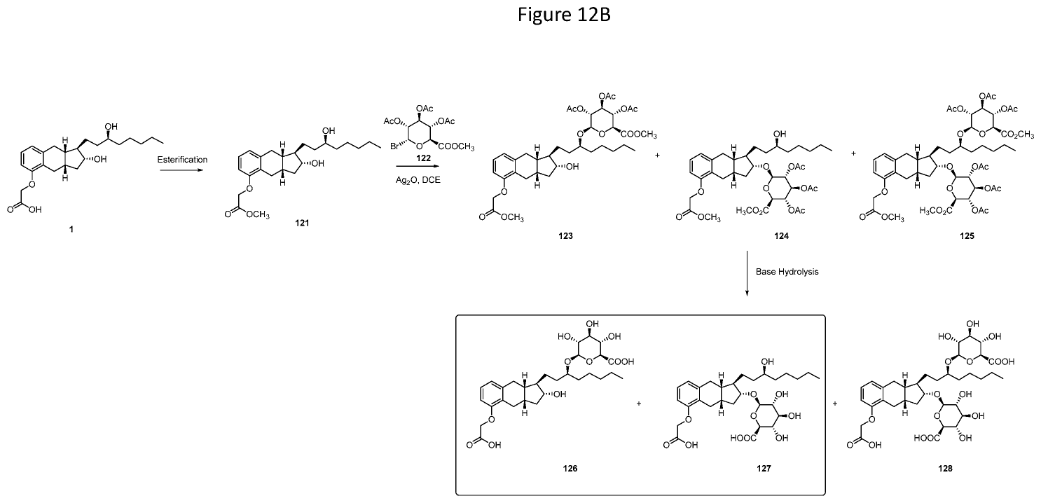

Scheme 11 in FIG. 12A-B schematically illustrates an exemplary route for synthesizing treprostinil analog 126 from treprostinil. FIG. 12A presents a general flow for Scheme 11, while FIG. 12B shows Scheme 11 with exemplary conditions.

Treprostinil 1 may be converted into compound 121 via esterification reaction. Compound 121 may be then converted into a mixture of compounds 123, 124 and 125. This mixture may be converted into a mixture of compounds 126, 127 and 128 via a base hydrolysis.

Compound 126 may be separated from the mixture using a separation technique such as an HPLC.

Embodiments described herein are further illustrated by, though in no way limited to, the following working examples.

### WORKING EXAMPLES

### 1. Syntheses of Treprostinil and Treprostinil Analog

### 1.1. Synthesis of Tricyclic Aldehyde Core

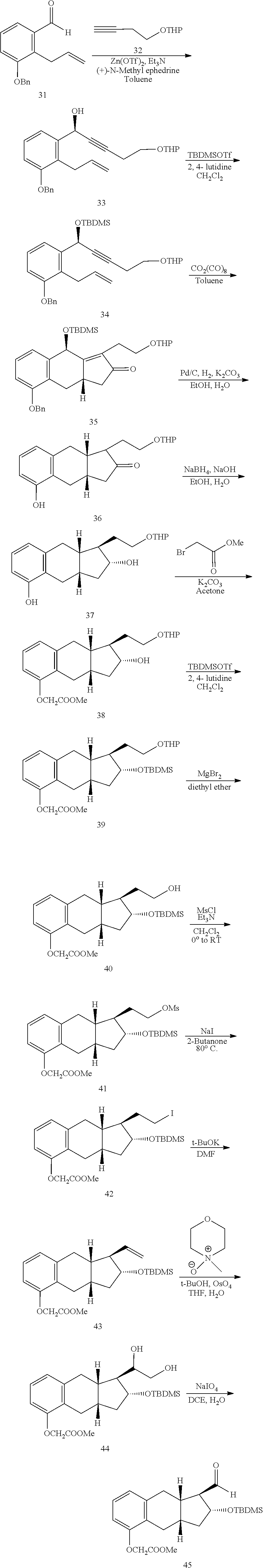

### Experimental

### Synthesis of Chiral Benzylalkynol (33)

To a solution of zinc trifluoromethanesulfonate (21.5 g, 0.99 mol) in toluene (50 mL) were added triethylamine (8.0 g, 0.0792 mol) and (1S,2R)-(+)-N-methylephedrine (10.6 g, 0.99 mol) and stirred at room temperature for 2 h. To this mixture was added 2-(3-butynyloxy)tetrahydro-2H-pyran (32) (12.2 g, 0.0792 mol) and stirred for 0.5 h. To this mixture was added a solution of 2-allyl-3-benzyloxybenzaldehyde (31) (5.0 g, 0.0198 mol) in toluene (10 mL). The reaction mixture was stirred at room temperature overnight. After completion, reaction was worked up to give the crude product (33) (60.2 g) as a viscous liquid. The crude product was purified by column chromatography to yield pure chiral benzylalkynol (33) (7.22 g).

### Synthesis of Benzylalkynol tert-Butyldimethylsilyl Ether (34)

To a solution of chiral benzylalkynol (33) (7.15 g, 0.0176 mol) in dichloromethane (80 mL) was added 2,6-lutidine (2.45 g, 0.0229 mol) and stirred at ambient temperature under argon. The mixture was stirred until a clear solution was obtained. The mixture was cooled and a solution of tert-butyldimethylsilyl trifluoromethanesulfonate (6.05 g, 0.0229 mol) in dichloromethane (40 mL) was added dropwise. After completion of the reaction, it was worked up to give the crude product (34) (9.6 g) as a viscous liquid. Another 7.15 g batch was combined and purified by column chromatography to give pure benzylalkynol tert-butyldimethylsilyl ether (34) (16.93 g).

### Synthesis of Tricyclic Enone (35)

To a solution of benzylalkynol tert-butyldimethylsilyl ether (34) (6.8 g, 0.0138 mol) in toluene (70 mL) was added dicobalt octacarbonyl (4.7 g, 0.0138 mol). This mixture was then heated to reflux, which continued for 2 h. The reaction was filtered through Celite and evaporated in vacuo to yield crude compound (35) (12.8 g) as a viscous liquid. The crude product was purified by column chromatography to yield tricyclic enone (35) (5.8 g).

### Synthesis of Tricyclic Ketone (36)

To a solution of tricyclic enone (35) (5.4 g, 0.0098 mol) in ethanol (80 mL) and water (2.7 mL), Pd/C (2.69 g) was added. This mixture was stirred under atmosphere of hydrogen gas. After 24 h, the reaction mixture was passed through Celite and the filtrate was evaporated in vacuo to yield crude tricyclic ketone (36) (5.1 g) as a viscous liquid. The crude product was purified by column chromatography to yield tricyclic ketone (36) (3.08 g).

### Synthesis of Tricyclic Alcohol (37)

To a solution of tricyclic ketone (36) (5.1 g, 0.015 mol) in ethanol (90 mL) was added a solution of sodium hydroxide (6.2 g, 0.155 mol) in water (20 mL) at −10° C. To this mixture, sodium borohydride (1.1 g, 0.031 mol) was added. The reaction mixture was stirred for 2 h and at this stage, the reaction was complete. The reaction mixture was worked up and concentrated in vacuo to give the crude (37) (4.95 g). The crude product was used as such for the next step without purification.

### Synthesis of Tricyclic Methyl Ester (38)

To a solution of tricyclic alcohol (37) (4.8 g, 0.0144 mol) in acetone (70 mL), potassium carbonate (4.0 g, 0.0288 mol) and methyl bromoacetate (2.2 g, 0.0158 mol) was added. The reaction mixture was stirred. After 72 h, the reaction was complete and the reaction mixture was filtered, and the filtrate was evaporated in vacuo to give the crude product (38) (6.3 g) as a viscous liquid. The crude product (6.3 g) was purified by column chromatography to yield tricyclic methyl ester (38) (5.5 g).

### Synthesis of Tricyclic tert-Butyldimethylsilyl Ether (39)

To a solution of tricyclic methyl ester (38) (2.8 g, 0.0069 mol) in dichloromethane (30 mL), 2,6-lutidine (1.1 g, 0.0103 mol) was added. The mixture was cooled and a solution of tert-butyldimethylsilyl trifluoromethanesulfonate (2.19 g, 0.0082 mol) in dichloromethane (20 mL) was added dropwise. The reaction was stirred for 1 h and at this stage, the reaction was complete. The reaction mixture was worked up to give the crude product (39) (4.2 g) as a viscous liquid. The crude product from another 2.8 g batch was combined and purified by column chromatography to yield tricyclic tert-butyldimethylsilyl ether (39) (6.39 g).

### Synthesis of Tricyclic tert-Butyldimethylsilyl Ether Alcohol (40)

To a solution of tricyclic tert-butyldimethylsilyl ether (39) (1.5 g, 0.0028 mol) in diethyl ether (30 mL), magnesium bromide (3.19 g, 0.0173 mol) was added. The reaction was stirred overnight and after 24 h, the reaction mixture was worked up to give the crude product (40) (1.6 g) as a viscous liquid. The crude product (1.6 g) was purified by column chromatography to yield tricyclic tert-butyldimethylsilyl ether alcohol (40) (1.1 g).

### Synthesis of Tricyclic Mesylate (41)

To a solution of tricyclic tert-butyldimethylsilyl ether alcohol (40) (1.1 g, 0.0026 mol) in dichloromethane (30 mL), triethylamine (0.58 g, 0.0057 mol) was added. To the cold mixture, methanesulfonyl chloride (0.36 g, 0.0031 mol) was added. After the addition, reaction mixture was stirred for 2 h. At this stage the reaction was complete. The reaction was worked up to give the crude tricyclic mesylate (41) (1.37 g). The crude product was carried as such in the next step without further purification.

### Synthesis of Tricyclic iodide (42)

To a solution of tricyclic mesylate (41) (1.37 g, 0.0027 mol) in 2-butanone (40 mL), sodium iodide (2.45 g, 0.0164 mol) was added. This reaction mixture was refluxed for 1.5 h. At this stage the reaction was complete. The reaction mixture was evaporated and worked up to give the crude tricyclic iodide (42) (1.2 g) as a viscous liquid. The crude product was carried as such in next step without further purification.

### Synthesis of Tricyclic alkene (43)

To a solution of tricyclic iodide (42) (2.34 g, 0.00429 mol) in N,N-dimethylformamide (35 mL) was added potassium tert-butoxide (2.97 g, 0.02646 mol) and stirred at room temperature for 2 h. The reaction mixture was worked up to obtain crude tricyclic alkene (1.98 g). To this crude product in acetone (40 mL) was added potassium carbonate (2.72 g, excess) and methyl iodide (2.79 g, excess), and stirred at room temperature overnight. This crude reaction mixture was passed through Celite and evaporated in vacuo to obtain crude tricyclic alkene (43) (1.3 g). The crude product was purified by column chromatography to yield tricyclic alkene (43) (0.85 g)

### Synthesis of Tricyclic diol (44)

To a solution of tricyclic alkene (43) (0.21 g, 0.00052 mol) in a mixture of tert-butanol (10 mL), tetrahydrofuran (3 mL) and water (1 mL) (ratio=10:3:1) was added osmium tetroxide (0.01 g, 0.00005 mol) and 4-methylmorpholine N-oxide (0.06 g, 0.00053 mol) and this was stirred under argon at room temperature. After 4 h, the reaction was complete, and the reaction mixture was worked up to obtain crude tricyclic diol (44) (0.14 g). This crude compound was carried as such in the next step without further purification.

### Synthesis of Tricyclic Aldehyde (45)

To a solution of tricyclic diol (44) (0.14 g, 0.00031 mol) in a mixture of 1,2-dichloroethane (10 mL) and water (10 mL) (ratio=1:1) was added sodium periodate (0.12 g, 0.00077 mol) at room temperature. After two overnights the crude reaction mixture worked up to obtain crude tricyclic aldehyde (45) (0.12 g). This crude compound was carried as such in the next step without further purification.

### 1.2. Synthesis of Treprostinil

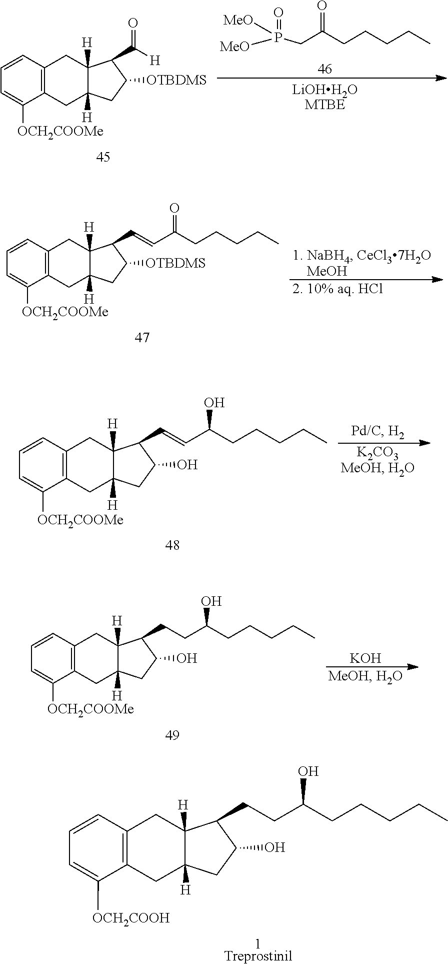

### Experimental

### Synthesis of Tricyclic Enone (47)

To a solution of dimethyl-2-oxoheptylphosphonate (46) (0.08 g, 0.0004 mol) in tert-butyl methyl ether (5 mL) was added lithium hydroxide monohydrate (0.01 g, 0.0004 mol) and stirred at room temperature for 1.5 h. To this mixture, a solution of tricyclic aldehyde (45) (0.12 g, 0.0002 mol) in tert-butyl methyl ether (5 mL) was added and stirred at room temperature for 4 h. The reaction was complete, and the mixture was worked up to obtain crude tricyclic α,β-unsaturated ketone (47) (0.14 g). The crude product was purified by column chromatography to yield pure tricyclic enone (47) (0.1 g).

### Synthesis of Tricyclic Alkenol (48)

To a solution of tricyclic α,β-unsaturated ketone (47) (0.1 g, 0.0002 mol) in methanol (10 mL) was added cerium(III) chloride heptahydrate (0.07 g, 0.0002 mol) at room temperature. This mixture was cooled and then sodium borohydride (0.007 g 0.0002) was added and stirred. After 1 h, the reaction was worked up to obtain crude product. This crude product was dissolved in methanol (10 mL) and 10% aq. HCl (0.3 mL) was added. This mixture was stirred at room temperature. After 1 h, the reaction was worked up to obtain crude tricyclic alkenol (48) (0.07 g). This crude compound was carried over in next step without further purification.

### Synthesis of Treprostinil methyl ester (49)

To a solution of tricyclic alkenol (48) (0.07 g) in a mixture of methanol (10 mL) and water (0.2 mL) were added Pd/C (0.1 g). This was stirred under hydrogen atmosphere overnight. The mixture was passed through Celite and evaporated in vacuo to yield crude treprostinil methyl ester (49) (0.07 g).

### Synthesis of Treprostinil (1)

To a solution of treprostinil methyl ester (49) (0.07 g, 0.0002 mol) in methanol (10 mL) was added a solution of potassium hydroxide (0.09 g, 0.0002 mol) in water (1 mL) and stirred overnight. The reaction mixture was worked up and evaporated in vacuo to obtain treprostinil (1) (45 mg) as solid. This treprostinil was characterized by ¹H NMR.

### 1.3. Synthesis of Treprostinil Analog

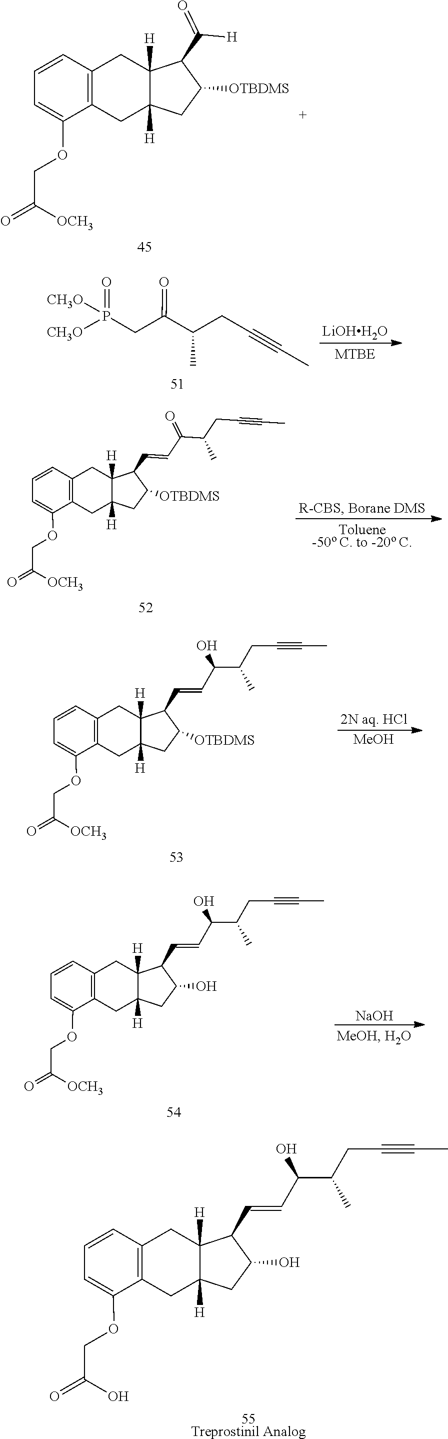

### Experimental

### Synthesis of Tricyclic Enone (52)

To a solution of phosphonate side chain (51) (100 mg, 0.432 mmol) in tert-butyl methyl ether (1 mL) was added lithium hydroxide monohydrate (17 mg, 0.396 mmol) and stirred at room temperature for 2 h. To this mixture, a solution of tricyclic aldehyde (45) (100 mg, 0.24 mol) in tert-butyl methyl ether (2 mL) was added and stirred at room temperature for 4 h. The reaction was worked up to obtain crude tricyclic enone (52) (132 mg). This was combined with another 301 mg batch and purified by column chromatography to yield pure tricyclic enone (52) (464 mg).

### Synthesis of Tricyclic Alkenol (53)

To a solution of borane dimethylsulfide in toluene (2.0M, 57 μL, 0.114 mmol) in toluene (0.3 mL) was added (R)-(+)-2-methyl-CBS-oxazaborolidine in toluene (1.0M, 114 μL, 0.114 mmol) and stirred for 1.5 h. This was cooled and a solution of tricyclic enone (52) (30 mg, 0.057 mmol) in toluene (0.7 mL) was added and this was stirred while allowing the temperature to ambient for 1.5 h. The reaction mixture was worked up to obtain crude product (53) (54 mg). This was purified by column chromatography to obtain tricyclic alkenol (53) (30 mg).

### Synthesis of Methyl Ester (54)

To a solution of tricyclic alkenol (53) (30 mg, 0.057 mmol) in methanol (1 mL) was added a 2.0N hydrochloric acid (72 μL, 0.142 mmol) and the mixture was stirred for 2 h. The reaction mixture was worked up and evaporated in vacuo to obtain crude methyl ester (54) (22.6 mg). This crude compound was carried over to next step without further purification.

### Synthesis of Treprostinil Analog (55)

To a solution of methyl ester (54) (18 mg, 0.0044 mol) in methanol (1 mL) was added a solution of sodium hydroxide (5.2 mg, 0.131 mmol) in water (0.2 mL) and stirred for 4 h. The crude reaction mixture was worked up and evaporated in vacuo to obtain treprostinil analog (55) (12.2 mg). This treprostinil analog was characterized by ¹H NMR.

### 1.3.2. Synthesis of Cyclohexyl Treprostinil Analog 60

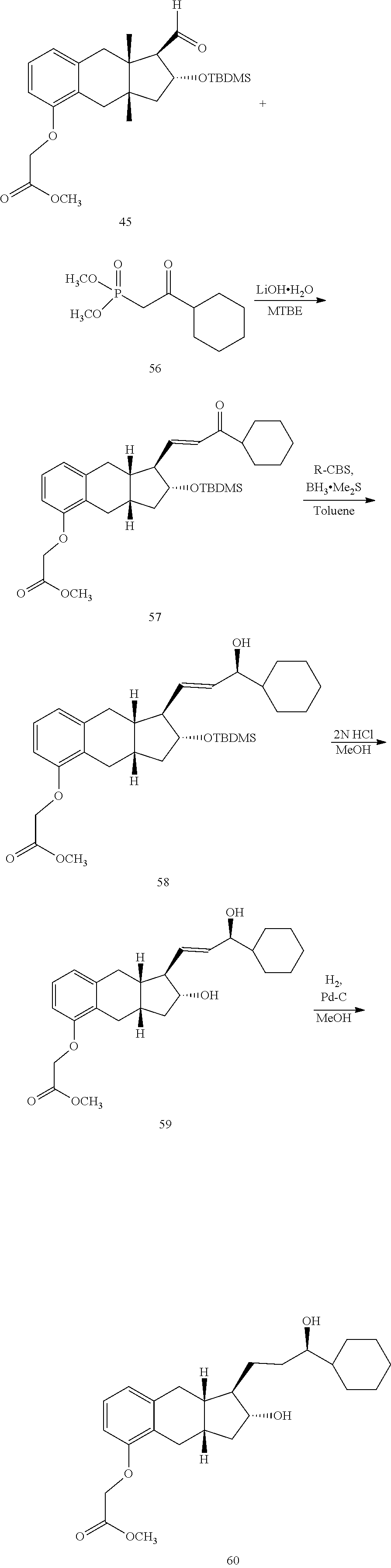

### Experimental

### Synthesis of Cyclohexyl Tricyclic TBDMS Enone (57)

To a solution of tricyclic TBDMS aldehyde (45) (0.093 g, 0.221 mmol, freshly prepared from corresponding alcohol) in anhydrous tert-butyl methyl ether (MTBE) (2 mL) was added a solution of cyclohexyl keto-phosphonate side chain (56) (0.078 g, 0.333 mmol) in anhydrous tert-butyl methyl ether (MTBE) (2 mL). The clear solution was stirred at room temperature under argon for 10 min and then added lithium hydroxide monohydrate (0.011 g, 0.262 mmol) in one portion. The reaction mixture was stirred at room temperature overnight and the reaction was complete. The mixture was quenched with water (10 mL) and separated the aqueous layer. The aqueous layer was extracted with MTBE (2×15 mL). The combined MTBE extracts were washed with water (2×15 mL), brine (1×5 mL), dried (Na₂SO₄), filtered and concentrated in vacuo to give viscous liquid (0.147 g). The crude product was purified by chromatography to give cyclohexyl tricyclic TBDMS enone (57) (0.052 g) and characterized by ¹H NMR, MS and 85.7% purity by HPLC.

### Synthesis of Cyclohexyl Tricyclic TBDMS Alkenol (58)

To a solution of (R)-(+)-2-methyl-CBS-oxazaborolidine in toluene (1.0 M) (0.20 mL, 0.20 mmol) and anhydrous toluene (1.0 mL) was added a solution of borane-methyl sulfide complex in toluene (2.0 M) (0.10 mL, 0.20 mmol) at room temperature under argon. The mixture was stirred at room temperature for 1 h and then cooled to −40±5° C. To this cold solution was added dropwise a solution of cyclohexyl tricyclic TBDMS enone (57) (0.05 g, 0.095 mmol) in anhydrous toluene (2.0 mL). The reaction mixture was allowed to warm to −20° C. over a period of 1 h and the reaction was complete. The mixture was cooled to −50±5° C. and then quenched by dropwise addition of anhydrous methanol (0.5 mL). The mixture was allowed to warm to 0° C. over a period of 1 h. The mixture was treated with saturated ammonium chloride (2.5 mL) and the mixture was stirred for 15 min. The mixture was filtered to remove the white solid and washed the solid with MTBE (3×10 mL). The combined filtrates transferred into a separatory funnel and separated the aqueous layer. The aqueous layer was extracted with MTBE (2×15 mL). The combined organic layers were washed with water (1×5 mL), brine (1×5 mL), dried (Na₂SO₄), filtered and concentrated in vacuo to give crude product as a colorless viscous liquid (0.085 g). The chromatography of the crude product gave pure cyclohexyl tricyclic TBDMS alkenol (58) (0.031 g) and characterized by IR, ¹H NMR, ¹³C NMR and MS.

### Synthesis of Cyclohexyl Tricyclic Hydroxy Alkenol (59)

To a solution of cyclohexyl tricyclic TBDMS alkenol (58) (0.03 g, 0.057 mmol) in methanol (1.5 mL) was added a solution of hydrochloric acid (2N) (0.08 mL, 0.16 mmol) at room temperature. The reaction mixture was stirred at room temperature for 30 min and the reaction was complete. The mixture was neutralized with saturated sodium hydrogen carbonate (2 mL) to pH 7-8, and the methanol was evaporated off in vacuo and the aqueous residue was treated with water (10 mL) and then extracted with ethyl acetate (3×10 mL). The combined ethyl acetate extracts were washed with water (2×10 mL), brine (1×5 mL), dried (Na₂SO₄), filtered and concentrated in vacuo to give cyclohexyl tricyclic hydroxy alkenol (59) as a light yellow viscous liquid (0.021 g, 87.5%) and characterized by ¹H NMR and purity by HPLC (92.2%).

### Synthesis of Cyclohexyl Tricyclic Methyl Ester (60)

To a solution of cyclohexyl tricyclic hydroxy alkenol (59) (0.01 g, 0.024 mmol) in methanol (1.5 mL) was added palladium on carbon, 5 wt %, 50% wet (0.01 g) at room temperature. The reaction mixture was stirred at room temperature under the atmosphere of hydrogen (filled in balloon) for 3.5 h. The reaction mixture was filtered through a pad of Celite (0.05 g) and washed the solid with methanol (3×2 mL). The filtrate was concentrated in vacuo to give cyclohexyl tricyclic methyl ester (60) (0.011 g) as off-white solid and characterized by ¹H NMR and LSMS.

### 2. Syntheses of Treprostinil Metabolites

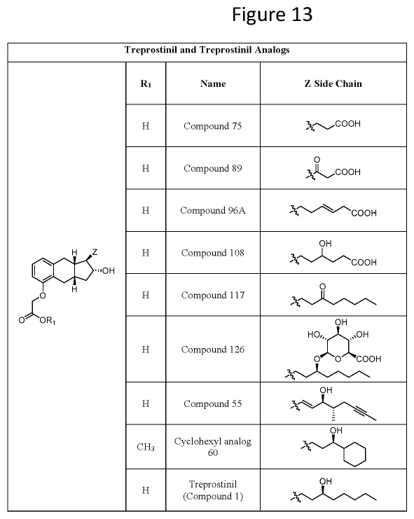

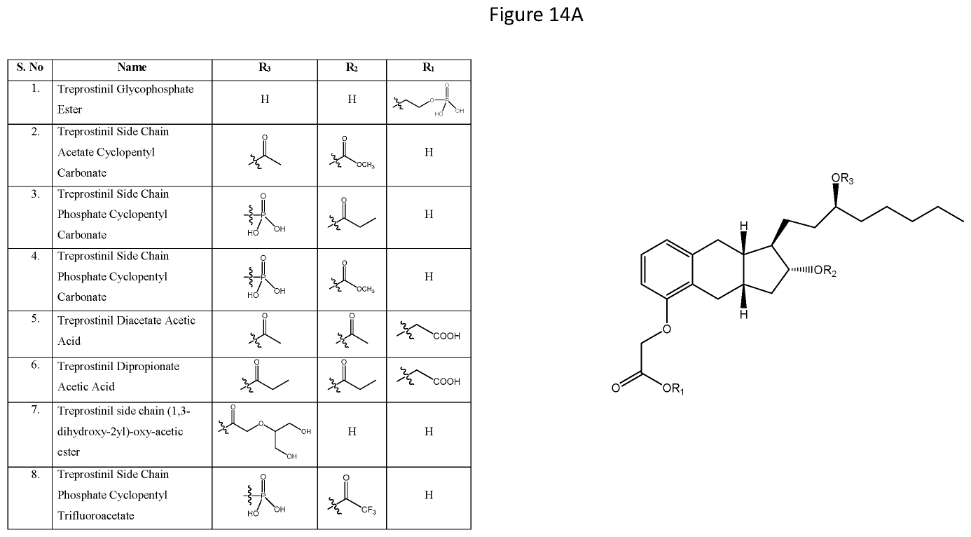

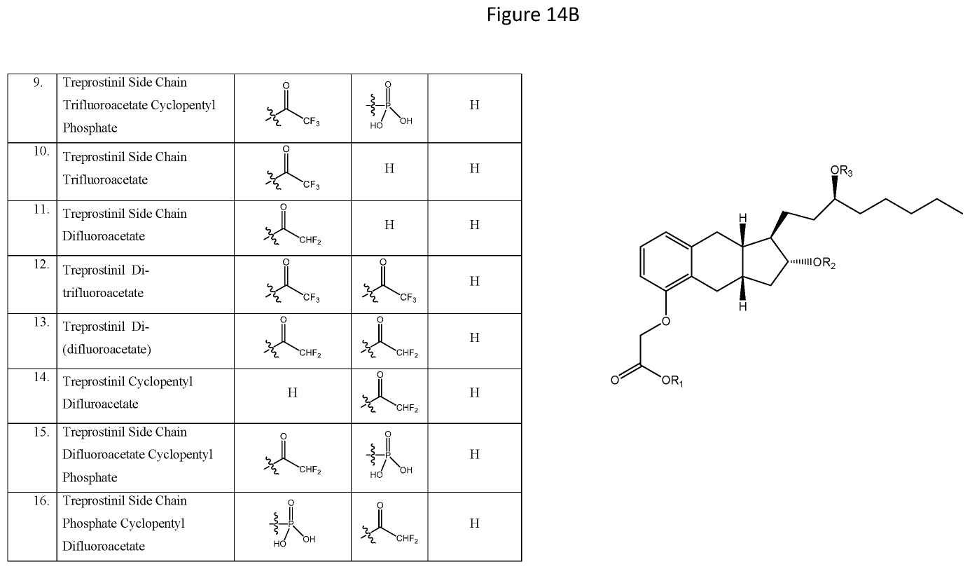

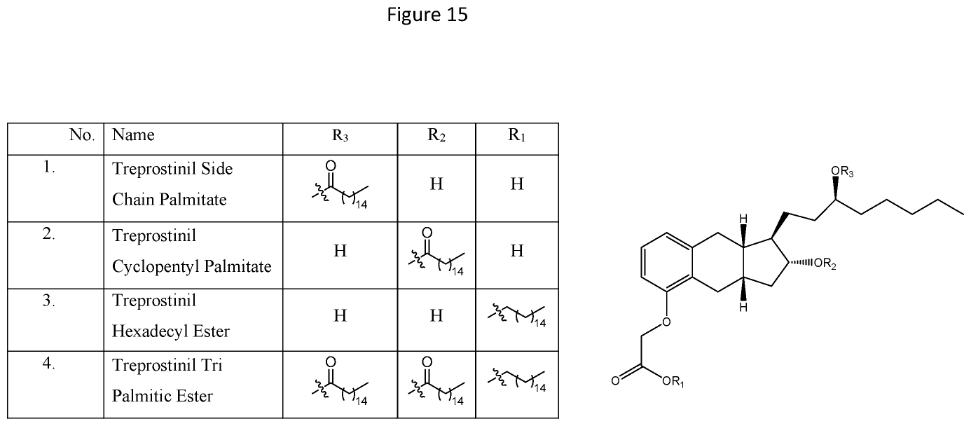

### 2.1. Synthesis of Compound 89 in FIG. 13

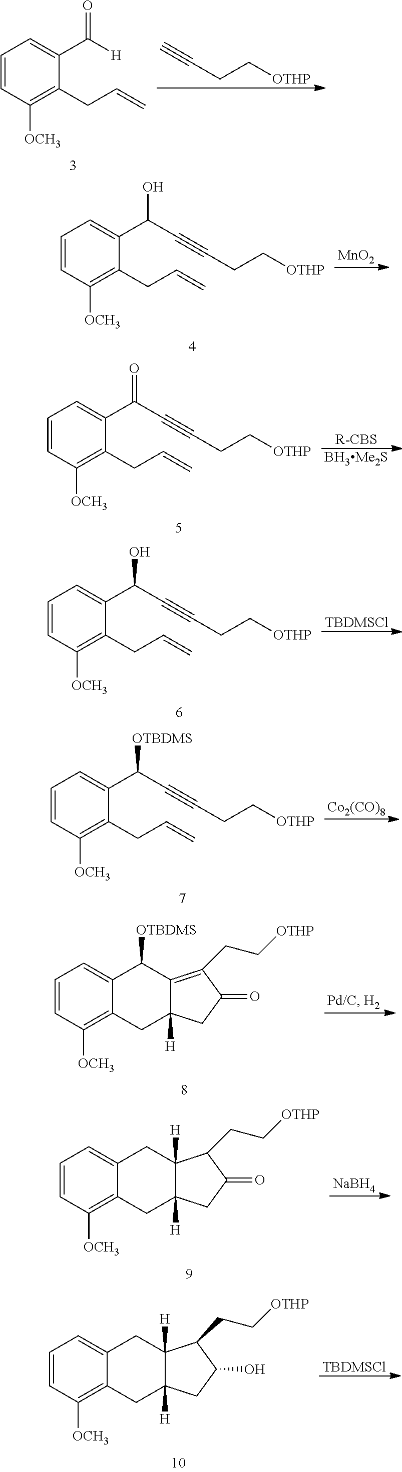

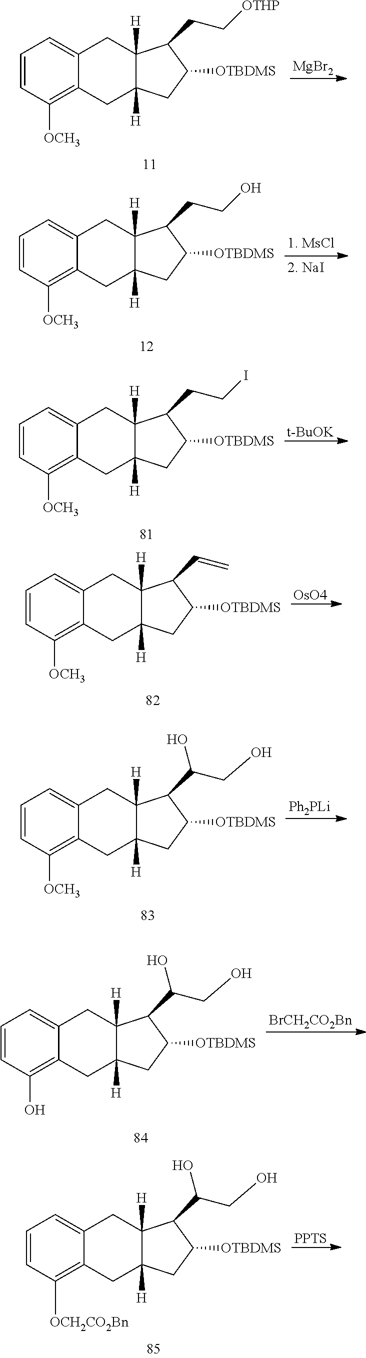

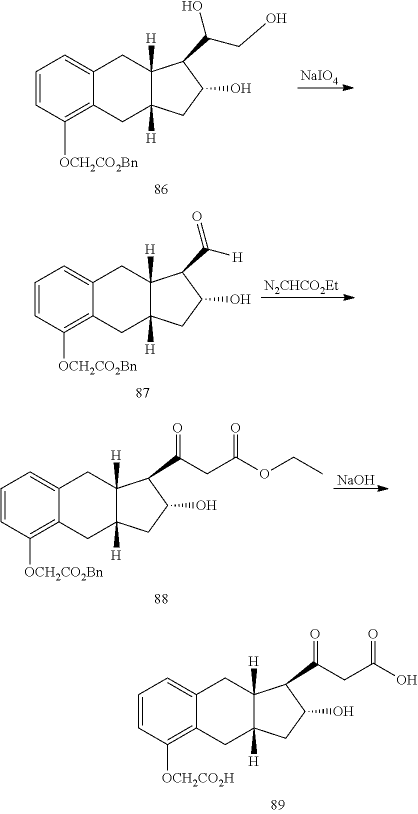

### Experimental

### Synthesis of Racemic Benzyl Alkynol (4)

To a solution of tetrahydro-2-(3-butynyloxy)-tetrahydro-2H-pyran (25.0 g, 162.1 mmol, 1.10 eq) in anhydrous tetrahydrofuran (200 mL) was added slowly ethylmagnesium bromide in ether (3.0M in ether) (54 mL, 133.3 mmol, 1.10 eq) under nitrogen at ambient temperature over a period of 30 min dropping of the reagent and gentle refluxing of the reaction mixture. After complete addition, the reaction mixture was allowed to cool to room temperature over a period of 1.5 h and then cooled to 0 to −10° C. To this cold solution was added slowly a solution of 2-allyl-3-methoxybenzaldehyde (3) (26.7 g, 147.4 mmol, 1.0 eq) in tetrahydrofuran (50 mL) keeping the temperature of the reaction mixture between 0 and 10° C. over a period of 30 min. The reaction mixture was allowed to warm to room temperature and stirred overnight. After 20 h, the reaction was checked by TLC. The mixture was quenched carefully with saturated ammonium chloride solution (˜20 mL) keeping the temperature of the mixture below 35° C. during the addition of the ammonium chloride solution. The white granulated mixture was stirred for 30 min, diluted with ethyl acetate (200 mL) and then filtered, washed with ethyl acetate (400 mL). The filtrate was concentrated in vacuo to get viscous liquid (51 g). The crude product was purified by silica gel column chromatography to get pure racemic benzyl alkynol (4) (42.5 g, 85%).

### Synthesis of Aryl Alkynyl Ketone (5)

To a solution of racemic benzylalkynol (4) (42.5 g, 129 mmol, 1.0 eq) in acetone (1,000 mL) was added manganese (IV) oxide (˜85% purity, 132.0 g, 1290 mmol, 10.0 eq) in portions with stirring at room temperature. The reaction mixture was stirred overnight. After 22 h, the reaction was checked by TLC. The mixture was filtered through Celite (55 g) in a sintered glass funnel and washed with acetone (1500 mL). The filtrate was concentrated in vacuo to get as a viscous liquid (40 g). The crude product was purified by silica gel column chromatography to get to aryl alkynyl ketone (5) as clear pale yellow viscous liquid (36 g, 85%).

### Synthesis of Chiral Benzylalkynol (6)

To a solution of (R) (+)-2-methyl-CBS-oxazoborolidine (1.0M in toluene) (130 mL, 130 mmol, 1.2 eq) was added a solution of aryl alkynyl ketone (5) (35 g, 107 mmol, 1.0 eq) in anhydrous tetrahydrofuran (300 mL) under nitrogen at room temperature. The mixture was stirred for 20 min and then cooled to −30° C., then borane methyl sulfide complex (16.8 g, 21 mL, 221 mmol, 2.1 eq) was added slowly keeping the temperature of the mixture between −40 and −30° C. The mixture was stirred at −30° C. for 2 h and then checked by TLC. The mixture was quenched carefully with methanol (22 mL) at −20 to −30° C. under nitrogen over a period of 20 min with stirring. The mixture was allowed to warm to room temperature and in 1 h. The mixture was treated with saturated ammonium chloride solution (˜100 mL) (until white granulated solids were formed). The mixture was filtered and washed with ethyl acetate (500 mL). The filtrate was concentrated in vacuo to get pale yellow viscous liquid with some white solid residue (35 g). The crude product was purified by silica gel column chromatography to get chiral benzylalkynol (6) as clear colorless viscous liquid (27.5 g, 78.1%).

### Synthesis of Benzyl Alkynyl tert-Butydimethylsilyl Ether (7)

To a solution of chiral benzylalkynol (6) (27.5 g, 83.2 mmol, 1.0 eq) in dichloromethane (300 mL) were added imidazole (8.6 g, 126 mmol, 1.5 eq), 4-(dimethylamino)pyridine (0.38 g, 3.1 mmol, 0.04 eq), tert-butyldimethylsilyl chloride (19.8 g, 131.4 mmol, 1.6 eq) and N,N′-dimethylformamide (5 mL) at room temperature. The reaction mixture was stirred at room temperature overnight. After 18 h, the reaction was checked by TLC. The mixture was washed with water (2×100 mL), brine (1×50 mL), dried over sodium sulfate, filtered, the filtrate was concentrated in vacuo to get almost pure benzyl alkynyl tert-butyldimethylsilyl ether (7) as clear viscous liquid (37 g, 100%). The product was used in the next step without further purification.

### Synthesis of Tricyclic THP-Ethyl Enone (8)

To a solution of benzyl alkynyl tert-butyldimethylsilyl ether (7) (37 g, 83.4 mmol, 1.0 eq) in dry toluene (390 mL) under nitrogen was added octacarbonyldicobalt (28.7 g, 83.9 mmol, 1.0 eq) in one portion at room temperature. The dark-brown reaction mixture was stirred at room temperature. Carbon monoxide was evolved slowly during stirring and the solution became reddish-brown after some time. The reaction was continued to stir for 2 h and checked by TLC and the complex was formed as a single spot. The mixture was refluxed (using pre-heated oil bath temperature of 120° C.) under nitrogen for 2 h and checked by TLC, there was no starting material. The reaction mixture was bubbled with air overnight at room temperature. The mixture was diluted with ethyl acetate (400 mL) followed by Celite (50 g). The mixture was filtered and washed with ethyl acetate (250 mL). The filtrate was concentrated in vacuo to dark-brown residue (36 g) The crude product was purified by silica gel column to get tricyclic ethyl ether enone (8) (25.3 g).

### Synthesis of Tricyclic THP Ethyl Ether Ketone (9)

To a solution of tricyclic ethyl ether enone (8) (0.42 g, purified and pretreated carbon, 0.89 mmol, 1.0 eq), in ethyl alcohol (8 mL) was added water (5 drops) potassium carbonate (0.021 g, 0.15 mmol, 0.17 eq, 5% w/w) and palladium, 5% (dry basis) on activated carbon, wet (50%), Degussa type (0.11 g, 25% w/w). The mixture was stirred under the atmosphere of hydrogen (filled in a balloon) at room temperature for 3 h and checked by TLC and IR and found complete. The mixture was filtered through a pad of Celite in a sintered glass funnel and washed with ethyl alcohol (20 mL). The hydrogenated product was used in the next step without isolation of tricyclic THP ethyl ether ketone (9).

### Synthesis of Tricyclic THP Ethyl Ether Alcohol (10)

To a solution of tricyclic THP ethyl ether ketone (9) (17.85 g, 51.83 mmol, 1.0 eq, calculated yield from previous step) in ethyl alcohol and water (˜540 mL) was added a solution of sodium hydroxide (20.73 g, 518.25 mmol, 10.0 eq dissolved in 180 mL of water) at −10° C. The mixture was stirred at −10° C. for 30 min and then added sodium borohydride (3.92 g, 103.7 mmol, 2.0 eq) in portions. After complete addition, the mixture was stirred at −10° C. for 15 min and allowed to warm to room temperature over a period of 1.5 h and then checked by TLC. The reaction mixture was quenched with saturated ammonium chloride (100 mL until pH changed from 14 to 9). The mixture was filtered through a pad of Celite and the filtrate was concentrated in vacuo to remove ethyl alcohol. The residue was dissolved in water (100 mL) and dichloromethane (150 mL). Separated the aqueous layer and then extracted with dichloromethane (2×70 mL). The dichloromethane extracts were washed with brine (50 mL), dried over sodium sulfate, filtered and concentrated in vacuo to get viscous liquid of tricyclic THP ethyl ether alcohol (10) (17.59 g). The product (10) was pure enough to use in the next step.

### Synthesis of Tricyclic Methoxy TBDMS THP Ethyl Ether (11)

To a solution of tricyclic alcohol (10) (8.57 g, 24.74 mmol, 1.0 eq) in dichloromethane (150 mL) were added imidazole (2.53 g, 37.16 mmol, 1.5 eq), 4-(dimethylamino)pyridine (0.06 g, 0.491 mmol, 0.02 eq) and tert-butyldimethylsilyl chloride (4.85 g, 32.18 mmol, 1.3 eq) at room temperature. The reaction mixture was stirred at room temperature overnight. After 18 h, the reaction was checked by TLC and complete. The mixture was washed with water (2×50 mL), brine (1×20 mL), dried over sodium sulfate and filtered; the filtrate was concentrated in vacuo to get almost pure tricyclic methoxy TBDMS THP ethyl ether (11) as clear viscous liquid (10.65 g, 93.40%). The pure compound (11) was characterized by ¹H and ¹³C NMR spectra. The product was used in the next step without further purification.

### Synthesis of Tricyclic Methoxy TBDMS Ethyl Alcohol (12)

To a solution of tricyclic methoxy TBDMS THP ethyl ether (11) (5.36 g, 11.63 mmol, 1.0 eq) in diethyl ether (200 mL) was added magnesium bromide (12.87 g, 69.90 mmol, 6.0 eq) at room temperature under nitrogen. The reaction mixture was stirred at room temperature overnight. After 16 h, the mixture was checked by TLC and complete. The reaction mixture was quenched carefully with water (100 mL). The ether layer was separated, and the aqueous layer was extracted with ethyl acetate (2×200 mL). The combined organic layers were washed with water (1×150 mL), brine (1×150 mL), dried over sodium sulfate, filtered and concentrated in vacuo to get tricyclic methoxy TBDMS ethyl alcohol (12) and was purified by silica gel chromatography to obtain a pale yellow viscous liquid (3.48 g, 80%). The compound (12) was characterized by ¹H and ¹³C NMR spectra.

### Synthesis of Tricyclic Methoxy TBDMS Ethyl Mesylate (81′)

To a solution of tricyclic methoxy TBDMS ethyl alcohol (12) (0.18 g, 0.48 mmol, 1.0 eq) in dichloromethane (10 mL) was added triethylamine (0.15 mL, 0.11 g, 1.10 mmol, 2.27 eq). The mixture was cooled to 0° C. and then added a solution of methanesulfonyl chloride (0.07 g, 0.58 mmol, 1.2 eq) in dichloromethane (1 mL). The reaction mixture was stirred at 0° C. and then allowed to warm to room temperature for a period of 1.5 h. The mixture was checked by TLC and complete. The reaction mixture was quenched with brine (10 mL) and separated the layer. The aqueous layer was extracted with dichloromethane (2×20 mL). The combined dichloromethane extracts were washed with brine (1×10 mL), dried over sodium sulfate, filtered and concentrated in vacuo to get tricyclic methoxy TBDMS ethyl mesylate (81′) as viscous amber color liquid (0.24 g, 100%). The mesylate was characterized by ¹H and ¹³C NMR spectra. The mesylate was used in the next step without further purification.

### Synthesis of Tricyclic Methoxy TBDMS Ethyl Iodide (81)

To a solution of crude mesylate (81′) (0.21 g, 0.46 mmol, 1.0 eq) in acetone (15 mL) was added sodium iodide (0.21 g, 1.40 mmol, 3.03 eq) in one portion at room temperature. The reaction mixture was stirred at room temperature overnight. After 42 h, the reaction was checked by TLC. There was a very little product, however, the mixture was quenched with saturated sodium bicarbonate (5 mL) and removed acetone in vacuo. The residue was extracted with dichloromethane (30 mL) and washed with saturated sodium bicarbonate (1×15 mL), dried over sodium sulfate, filtered and concentrated in vacuo to get the product as viscous liquid. The product was analyzed by ¹H NMR and found to be a mixture of tricyclic methoxy TBDMS ethyl mesylate (81′) (major) and tricyclic methoxy TBDMS ethyl iodide (81) as viscous liquid (minor) (0.19 g).

The recovered mesylate (81′) (0.18 g, 0.39 mmol, 1.0 eq) was dissolved in 2-butanone (20 mL) and added sodium iodide (0.35 g, 2.33 mmol, 6.0 eq) in one portion at room temperature. The reaction mixture was gently heated to reflux for 3 h. The mixture was checked by TLC (EtOAc/Hexanes, 3:7) and found complete The mixture was cooled to room temperature and then removed 2-butanone in vacuo. The residue was dissolved in water (10 mL) and then extracted with ethyl acetate (3×10 mL). The combined ethyl acetate extracts were washed with saturated sodium bicarbonate (1×10 mL), 10% sodium thiosulfate solution (1×10 mL), brine (1×10 mL), dried over sodium sulfate, filtered and concentrated in vacuo to get tricyclic methoxy TBDMS ethyl iodide (81) as pale yellow viscous liquid and solidified on standing at room temperature (0.17 g, 89%). The compound (81) was characterized by ¹H and ¹³C NMR spectra. The product was used in the next step without further purification.

### Synthesis of Tricyclic Methoxy TBDMS Ethylene (82)

To a solution of tricyclic methoxy TBDMS ethyl iodide (81) (0.14 g, 0.29 mmol, 1.0 eq) in tert-butanol (15 mL) was added potassium tert-butoxide (0.07 g, 0.62 mmol, 2.14 eq) in one portion at room temperature (slightly exothermic!). The reaction mixture was gently heated to reflux for 1 h and then checked by TLC. The reaction seemed to be complete. The mixture was quenched with water (10 mL) then removed tert-butanol in vacuo. The residue was extracted with ethyl acetate (20 mL), washed with water (2×10 mL), brine (1×10 mL), dried over sodium sulfate, filtered and concentrated in vacuo to get viscous liquid which solidified on standing (0.13 g). The ¹H NMR of the product showed mostly starting material (iodide) along with a very little product, tricyclic methoxy TBDMS ethylene (82).

The above recovered tricyclic methoxy TBDMS ethyl iodide (81) (0.13 g, 0.27 mmol, 1.0 eq) was dissolved in N,N′-dimethylformamide (10 mL) was added potassium tert-butoxide (0.16 g, 1.42 mmol, 5.26 eq) in one portion at room temperature. The reaction mixture became brown (slightly exothermic!). The mixture was stirred at room temperature overnight. After 16 h, the reaction mixture was checked by TLC and complete. The mixture was quenched with saturated ammonium chloride (20 mL) and stirred the mixture for 1 h and then extracted with ethyl acetate (3×20 mL). The combined ethyl acetates were washed with saturated ammonium chloride (3×15 mL), brine (1×10 mL), dried over sodium sulfate, filtered and concentrated in vacuo to get tricyclic methoxy TBDMS ethylene (82) as light yellowish-brown liquid (0.08 g, 82%). The compound (82) was characterized by ¹H and ¹³C NMR spectra. The product was used in the next step without further purification.

### Synthesis of Tricyclic Methoxy TBDMS Ethane Diol (83)

To a solution of tricyclic methoxy TBDMS ethylene (82) (0.24 g, 0.67 mmol, 1.0 eq) in a mixture of tert-butanol (10 mL) and tetrahydrofuran (3 mL) and water (1 mL) (ratio=10:3:1) was added osmium tetroxide (0.017 g, 0.067 mmol, 0.1 eq) followed by 4-methymorpholine N-oxide (0.08 g, 0.68 mmol, 1.01 eq). The reaction mixture was covered by aluminum foil and stirred at room temperature overnight. After 16 h, the reaction mixture was checked by TLC and complete. The mixture was treated with sodium thiosulfate solution (1M, 10 mL). The orange-red solution was stirred at room temperature for 1 h. The mixture was extracted with dichloromethane (3×20 mL). The combined organic extracts were washed with brine (1×20 mL), dried over sodium sulfate, filtered and concentrated in vacuo to get tricyclic methoxy TBDMS ethane diol (83) as viscous liquid (0.29 g, 100%). The compound (83) was characterized by ¹H and ¹³C NMR spectra. The crude product was used as such in the next step without further purification.

### Synthesis of Tricyclic Hydroxy TBDMS Ethane Diol (84)

To a solution of tricyclic methoxy TBDMS ethane diol (83) (0.55 g, 1.40 mmol, 1.0 eq) in anhydrous tetrahydrofuran (15 mL) was added n-butyllithium in hexane (2.5M in hexane, 2.8 mL, 7.0 mmol, 5.0 eq) at −20° C. under nitrogen followed by diphenylphosphine (10 wt. % in hexane, 10.4 mL, 5.58 mmol, 4.0 eq). The reaction mixture (deep orange red) was stirred −20° C. for 20 min and then allowed the mixture to room temperature over a period of 1 h. The mixture was gently heated to reflux overnight. After 16 h, the reaction mixture was checked by TLC and was not complete. Additional n-butyllithium in hexanes (2.5M, 2.6 mL) and diphenylphosphine (10 wt. % in hexane, 8.0 mL) were added. The reaction mixture was continued to reflux overnight. After 18 h, the reaction mixture was checked by TLC and was complete. The reaction mixture was quenched with saturated ammonium chloride (20 mL). The mixture was extracted with ethyl acetate (3×25 mL). The combined ethyl acetate layers were washed with water (1×30 mL), brine (1×10 mL), dried over sodium sulfate, filtered and concentrated in vacuo to get clear viscous liquid (1.07 g). The crude product was purified by silica gel column chromatography to get tricyclic hydroxy TBDMS ethane diol (84) as viscous liquid, solidified on standing (0.19 g, 36%). The pure compound (84) was characterized by ¹H and ¹³C NMR spectra.

### Synthesis of Tricyclic TBDMS-Benzyloxycarbonylmethyl Ether Ethane Diol (85)

To a solution of tricyclic hydroxy TBDMS ethane diol (84) (1.75 g, 4.62 mmol, 1.0 eq) in acetone (100 mL) was added powdered potassium carbonate (1.40 g, 10.13 mmol, 2.20 eq) and benzyl 2-bromoacetate (1.27 g, 5.54 mmol, 1.20 eq). The mixture was gently heated to reflux overnight. After 18 h, the reaction mixture was checked by TLC and complete. The mixture was cooled to room temperature, then filtered and washed with acetone. The filtrate was concentrated in vacuo to get tricyclic TBDMS-benzyloxycarbonylmethyl ether ethane diol (85) as viscous liquid (2.47 g, 100%). The compound (85) was characterized by ¹H and ¹³C NMR spectra. The crude product was used as such without further purification in the next step.

### Synthesis of Tricyclic Benzyloxycarbonylmethyl Ether Triol (86)

To a solution of tricyclic TBDMS-benzyloxycarbonylmethyl ether ethane diol (85) (2.46 g, crude calculated as 2.43 g, 4.61 mmol, 1.0 eq) in acetone (100 mL) and water (5 mL) was added pyridinium p-toluenesulfonate (0.70 g, 4.63 mmol, 1.0 eq) at room temperature. The reaction mixture was stirred at room temperature overnight. After 68 h, the reaction was checked by TLC. The reaction was not complete, additional one equivalent of pyridinium p-toluenesulfonate (0.70 g, 4.63 mmol, 1.0 eq) was added. The mixture was continued to reflux overnight. After 16 h, the reaction mixture was checked by TLC and complete. The reaction was concentrated in vacuo to remove acetone. The aqueous layer was extracted with ethyl acetate (3×40 mL). The combined ethyl acetate extracts were washed with water (1×40 mL), brine (1×20 mL), dried over anhydrous sodium sulfate, filtered and concentrated in vacuo to get viscous liquid (1.92 g). The crude product was purified by silica gel column chromatography to get tricyclic benzyloxycarbonylmethyl ether triol (86) as viscous liquid/semi-solid (0.97 g, 51%).

### Synthesis of Tricyclic Benzyloxycarbonylmethyl Ether Hydroxy Aldehyde (87)

To a solution of tricyclic benzyloxycarbonylmethyl ether triol (86) (0.96 g, 2.33 mmol, 1.0 eq) in 1,2-dichloroethane (40 mL) was added a solution of sodium (meta)periodate (1.0 g, 4.67 mmol, 2.0 eq) in water (40 mL) at room temperature The reaction mixture was stirred overnight. After 16 h, the reaction mixture was checked by TLC and complete. The reaction mixture was a white emulsion. To the mixture was added water (70 mL) and dichloromethane (70 mL) and then added sodium chloride until the aqueous layer was saturated. The organic layer was separated and the aqueous layer was extracted with dichloromethane (3×30 mL). The combined dichloromethane extracts were washed with brine (1×20 mL), dried over sodium sulfate, filtered through a pad of silica gel in a sintered glass funnel. The compound was purified by silica gel column chromatography to get tricyclic benzyloxycarbonylmethyl ether hydroxy aldehyde (87) as clear viscous liquid (0.54 g, 61%). The compound (87) was characterized by ¹H NMR spectrum.

### Synthesis of Tricyclic Benzyloxycarbonylmethyl ether j-keto Ethyl Ester (88)

To a solution tricyclic benzyloxycarbonylmethyl ether hydroxy aldehyde (87) (0.53 g, 1.39 mmol, 1.0 eq) in dichloromethane (25 mL) were added ethyl diazoacetate (0.32 g, 2.80 mmol, 2.0 eq) and tin (II) chloride (0.06 g, 0.37 mmol, 0.27 eq) at room temperature. The reaction mixture was stirred at room temperature overnight. After 16 h, the reaction mixture was checked by TLC and complete. The reaction mixture was directly passed through silica gel column using neat dichloromethane followed by a mixture of ethyl acetate in hexanes (20-40%) to pure tricyclic benzyloxycarbonylmethyl ether j-keto ethyl ester (88) as clear viscous liquid (0.32 g) and slightly impure tricyclic benzyloxycarbonylmethyl ether j-keto ethyl ester (88) (0.19 g). Both pure and slightly impure j-keto ethyl ester (88) were characterized by ¹H NMR spectrum.

### Synthesis of Compound 89 in FIG. 13

To a solution of pure tricyclic benzyloxycarbonylmethyl ether 3-keto ethyl ester (88) (0.32 g, 0.68 mmol, 1.0 eq) in tetrahydrofuran (20 mL) was added a solution of 1.0M sodium hydroxide (4.1 mL, 4.1 mmol, 6.03 eq) at room temperature. The reaction mixture was stirred at room temperature overnight. After 66 h, the reaction mixture was diluted with water (20 mL) and then removed tetrahydrofuran in vacuo. The aqueous layer was extracted with dichloromethane (2×20 mL) to remove organic impurities. The aqueous layer was carefully acidified with 10% hydrochloric acid to pH 2-3 and extracted with ethyl acetate (3×25 mL). The combined ethyl acetate extracts were washed with brine (1×15 mL), dried over sodium sulfate, filtered and concentrated in vacuo to get compound 89 as pale yellow viscous liquid (0.24 g, 100%). The metabolite 89 was checked by ¹H NMR and confirmed by HPLC.

(i) Using Sodium hydroxide. Under similar condition, tricyclic benzyloxycarbonylmethyl ether 3-keto ethyl ester (88) (0.017 g, 0.036 mmol, 1.0 eq) in tetrahydrofuran (1.0 mL) was hydrolyzed with a solution of 1.0M sodium hydroxide (0.22 mL, 0.22 mmol, 6.04 eq) at room temperature to get compound 89 as pale yellow viscous liquid/semi-solid (0.01 g, 77%). The metabolite was checked by ¹H NMR spectrum and confirmed by HPLC.

(ii) Using Lithium hydroxide. Under similar condition, tricyclic benzyloxycarbonylmethyl ether β-ketoester (88) (0.017 g, 0.036 mmol, 1.0 eq) in tetrahydrofuran (1.0 mL) was hydrolyzed with a solution of 1.0M lithium hydroxide (0.22 mL, 0.22 mmol, 6.04 eq) at room temperature to get compound 89 as pale yellow viscous liquid/semi-solid (0.01 g, 77%). The metabolite was checked by ¹H NMR spectrum and confirmed by HPLC.

(iii) Using Barium hydroxide. Under similar condition, tricyclic benzyloxycarbonylmethyl ether β-keto ethyl ester (88) (0.017 g, 0.036 mmol, 1.0 eq) in tetrahydrofuran (1.0 mL) was hydrolyzed with a solution of 1.0M barium hydroxide (0.04 g, 0.23 mmol, 6.41 eq) at room temperature to get compound 89 as pale yellow viscous liquid/semi-solid (0.01 g, 77%). The metabolite was checked by ¹H NMR spectrum and confirmed by HPLC.

### 2.2. Synthesis of Compounds 75 and 108 in FIG. 13

### 2.2.1. Synthesis of Common Intermediate (12a) for Compounds 75 and 108 in FIG. 13

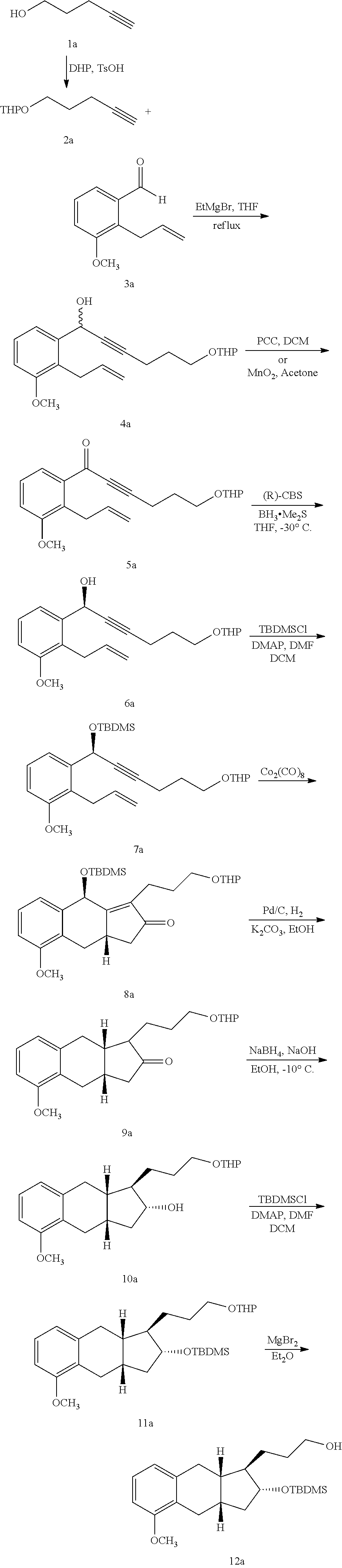

### Experimental

### Synthesis of 1-Pentyn-O-tetrahydropyran-2-yl-5-ol (Intermediate 2a)

A 500-mL, three-necked, round-bottom flask equipped with a mechanical stirrer and an argon inlet-outlet adapter connected to a bubbler was charged with 4-pentyn-1-ol (1a, 20 g, 0.238 moles), dichloromethane (200 mL), 3,4-dihydro-2H-pyran (21 g, 0.250 moles) and pyridinium p-toluenesulfonate (PPTS, 5.98 g, 0.024 moles) under argon at room temperature. The reaction mixture was stirred overnight at room temperature. The progress of reaction was monitored by TLC. After completion of the reaction, the mixture was washed with water (1×250 mL), brine (1×250 mL), dried over anhydrous sodium sulfate, filtered, and concentrated in vacuo to get the crude product 2a (39 g, Yield, 98%). The crude material was used as such in next step.

### Synthesis of Intermediate 4a

A 1-L, three-necked, round-bottom flask equipped with a mechanical stirrer, an addition funnel, a thermocouple and an argon inlet-outlet adapter connected to a bubbler was charged with 1-pentyn-O-tetrahydropyran-2-yl-5-ol (2a) (31.5 g, 0.187 moles), and anhydrous tetrahydrofuran (200-300 mL). Ethyl magnesium bromide (3M solution in diethyl ether) (62.4 mL, 0.187 moles) was added to the solution of 2a under argon keeping the temperature below 40° C. over a period of 20-30 minutes. After complete addition, the reaction mixture was stirred between 30° C. and 45° C. for 2 h. The reaction mixture was cooled to 0° C. (ice-water bath), and a solution of 2-allyl-3-methoxybenzaldehyde (3a) (30.0 g, 0.170 moles) in anhydrous tetrahydrofuran (50 mL) was added over a period of 10 minutes. The reaction mixture was stirred overnight at room temperature and the temperature of the reaction mixture allowed to rise to ambient temperature. After 16 h, the reaction was complete. At ambient temperature, the reaction mixture was quenched with saturated ammonium chloride, which resulted in suspension of granular solid. The mixture was filtered to remove granular inorganic solid, and the filtrate was concentrated in vacuo to get a crude product 4a (59 g). The crude product was purified by column chromatography to get pure 3-methoxy-2-(2-propenyl)-□-[5-[(tetrahydro-2H-pyran-4-yl)oxy]-1-pentnyl]benzene-methanol (intermediate 4a) as pale-yellow, viscous liquid (46.3 g, Yield, 79%).

### Synthesis of Intermediate 5a

A 2-L, two-necked, round-bottom flask equipped with a mechanical stirrer, a thermocouple was charged with intermediate 4a (46.0 g, 0.134 moles), dichloromethane (500 mL), Celite (40 g), and sodium acetate (22 g, 0.268 moles). The stirred suspension was cooled to 0° C., pyridinium chlorochromate (PCC, 57.6 g, 0.238 moles) was added while stirring. The reaction mixture was slowly allowed to warm to ambient temperature and stirring was allowed for 5 h. The mixture was filtered through a pad of Celite using a Buchner funnel. The dark-brown, gummy solid in the reaction flask and on the Buchner funnel were washed with ethyl acetate to recover maximum product. The solvent was removed in vacuo, and the crude product was purified by column chromatography to get 1-[3-methoxy-2-(2-propenyl)phenyl]-6-[(tetrahydro-2H-pyran-4-yl)oxy]-2-hexyn-1-one (intermediate 5a) as a light-yellow, viscous oil (30.67 g, yield, 67%)

### Synthesis of Intermediate 6a

A 1-L, three-necked, round-bottom flask equipped with a mechanical stirrer, a thermocouple, and an argon inlet-outlet trap was charged with aryl intermediate 5a (21 g, 0.061 moles), and anhydrous tetrahydrofuran (200-300 mL). A solution of (R)-methyl oxazaborolidine (1.0 M in toluene, 73.6 mL, 0.074 moles) was added under argon at room temperature. The mixture was cooled to −30° C. (dry ice/acetone-bath), and borane-methyl sulfide complex (9.32 mL, 0.122 moles) was added slowly keeping the temperature between −25° C. and −30° C. After complete addition, the reaction mixture was stirred for 1 h at the same temperature. The reaction was monitored by TLC. The reaction mixture was carefully quenched by slow addition of methanol over a period of 20 minutes keeping the temperature of exothermic reaction between −10° C. and −15° C. The reaction mixture was allowed to warm up to room temperature. A solution of 5% aqueous ammonium chloride was added with stirring (no exotherm was observed!). The organic layer was separated, and the aqueous layer was extracted with ethyl acetate (3×100 mL). The combined organic layers were washed with saturated sodium chloride (100 mL), dried over anhydrous sodium sulfate (˜10 g), filtered and concentrated in vacuo to get crude product. The crude product was purified by column chromatography to afford intermediate 6a (a semi-solid product) (11 g, yield, 52%).

### Synthesis of Intermediate 7a

A 1-L, three-necked, round-bottom flask equipped with a mechanical stirrer, a thermocouple, and an argon inlet-outlet trap was charged with a solution of chiral benzylalkynol (6a) (10.60 g, 30.77 mmol), dichloromethane (150 mL), imidazole (2.93 g, 43.08 mmol), and dimethylformamide (2 mL) at room temperature under argon. The mixture was stirred until a clear solution was obtained. The t-Butyldimethylsilyl chloride (6.03 g, 40.00 mmol) was added while keeping the temperature below 20° C. The reaction was stirred overnight. The temperature of the reaction mixture was allowed to rise to ambient temperature. After ˜16 h, the progress of the reaction was monitored by TLC. The mixture was washed with water (3×150 mL) and saturated sodium chloride (1×150 mL). It was dried over anhydrous sodium sulfate (10 g), filtered, and concentrated in vacuo to get the crude product 7a as viscous oil. The crude product was purified by column chromatography to yield benzyl alkynyl-t-butyldimethylsilyl ether (intermediate 7a), colorless viscous oil (12.66 g, 90%).

### Synthesis of Intermediate 8a

A 1-L, three-necked, round-bottom flask equipped with a mechanical stirrer, a thermocouple, and an argon inlet-outlet trap was charged with a solution of benzyl alkynyl t-butyldimethylsilyl ether (7a, 12 g, 26.16 mmol) in dichloromethane (120-150 mL) under argon. At room temperature, octacarbonyldicobalt (8.947 g, 26.16 mmol) was added, and the reaction mixture was stirred at ambient temperature. Carbon monoxide was evolved slowly, and the solution was turned reddish-brown after some time. Stirring was continued for 2 h. Dichloromethane was distilled from the reaction mixture in vacuo using a water bath (temperature of water-bath not exceeding 30° C.). The resulting brown, viscous liquid was dissolved in acetonitrile and transferred back to the 1-L, three-necked, round-bottom flask equipped with a mechanical stirrer, a thermocouple, an argon inlet-outlet trap and a condenser. The solution was heated at reflux under argon for 2 h. The reaction mixture was cooled to room temperature, and air was bubbled through the mixture overnight. After completion of the reaction, the reaction mixture was diluted with saturated ammonium chloride solution and the mixture was extracted with ethyl acetate (3×150 mL). The combined organic layers were washed with brine (1×150 mL), dried over anhydrous sodium sulfate (10 g). The organic phase was filtered. The filtrate was concentrated in vacuo to yield crude product 8a, brown oil. The crude product 8a was purified by column chromatography to yield pure tricyclic enone (8a) as light-brown oil (12 g, yield, 94%). The crude material was dissolved in ethanol (200 mL). To the solution, activated carbon (1.2 g) was added. The suspension was heated to reflux and hot solution was filtered through a pad of Celite. The filtrate was used as such in next step.

### Synthesis of Intermediate 9a

A 500-mL, three-necked, round-bottom flask equipped with a magnetic stirrer, and a hydrogen filled balloon, was charged with a solution of intermediate 8a (10.9 g) in absolute ethanol (100-150 mL, from previous step), anhydrous potassium carbonate (0.5 g) and 5% palladium on activated charcoal (1.98 g, 10%, 50% wet). The air in reaction flask was removed by vacuum and vacuum was replaced by hydrogen from attached balloon. This step was repeated three times. The mixture was hydrogenated under balloon pressure for 16 h (overnight) at ambient temperature. The progress of the reaction was monitored by TLC. Reaction was not completed, for this reason, the reaction mixture was filtered through a pad of Celite and pad of Celite was washed with more ethanol to recover material. The volume of reaction mixture was reduced to half by evaporation under vacuo. The reaction with same amount of reagents was repeated. After 16 hours, TLC indicated the absence of compound 8a (starting material). The mixture was filtered through a pad of Celite. The filtrate was concentrated in vacuo to get a colorless, viscous oil of crude product 9a. The crude product 9a was purified by column chromatography using 230-400 mesh silica gel. A solvent gradient of ethyl acetate in hexanes (0-20%) was used to elute the product from the column. The fractions containing the desired product are evaporated in vacuo to yield pure product of tricyclic ketone (9a) as colorless, viscous oil (6.38 g, yield, 74%)

### Synthesis of Intermediate 10a

A 500-mL, three-necked, round-bottom flask equipped with a mechanical stirrer was charged with a solution of tricyclic ketone (9a, 17.30, 17.3 mmol) in ethanol. The solution was cooled to −4° C. and 20% aqueous sodium hydroxide solution (6.92 g, 173.00 mmol, dissolved in 30 mL of water) was added with stirring over a period of 10-15 minutes. The reaction mixture was stirred for an additional 0.5 h and then sodium borohydride (700 mg, 18.52 mmol) was added. The stirring was continued at −10° C. for 2 h. After 2 h, an additional equivalent of sodium borohydride (610 mg, 16.14 mmol) was added, and stirring was continued for 2 h at −10° C. The progress of the reaction was monitored by TLC. The reaction mixture was quenched carefully by drop wise addition of glacial acetic acid (˜12 mL) until pH 5-6 was obtained. The mixture was allowed to attain ambient temperature. The undesired solid inorganic impurities are removed by filtration, and the filtrate was concentrated in vacuo. The residue was obtained after evaporation was dissolved in ethyl acetate (300 mL), and the resulting solution was stirred for 15 minutes. The ethyl acetate solution of compound 10a was washed with sodium bicarbonate (2×100 mL). The aqueous layer was extracted with ethyl acetate (2×150 mL). The combined organic extracts are washed with saturated sodium chloride solution (100 mL), dried over anhydrous sodium sulfate, filtered and concentrated in vacuo to get the crude product (tricyclic alcohol 10a) as oil (6.3 g, yield, ˜100%).

### Synthesis of intermediate 11a

A 100-mL, one-necked, round-bottom flask equipped with a magnetic stirrer was charged with a solution of crude Intermediate 10a (1.250 g, 3.47 mmol), dichloromethane (20 mL), imidazole (473 mg, 6.95 mmol), and dimethylformamide (0.5 mL) at room temperature under argon. The mixture was stirred until a clear solution was obtained. t-Butyldimethylsilyl chloride (1.045 g, 6.93 mmol) was added at room temperature. The reaction was stirred overnight. After ˜16 h, the progress of the reaction was monitored by TLC. The mixture was washed with water (1×50 mL) and saturated sodium chloride (1×50 mL). It was dried over anhydrous sodium sulfate, filtered, and concentrated in vacuo to get the crude product 11a as viscous oil. The crude product was purified by column chromatography to yield intermediate 11a, colorless viscous oil (1.45 g, 88%).

### Synthesis of Intermediate 12a

A 100-mL, one-necked, round-bottom flask equipped with a magnetic stirrer was charged with a solution of Intermediate 11a (1.40 g, 2.95 mmol), diethyl ether (50 mL), magnesium bromide (3.26 g, 17.70 mmol) at room temperature under nitrogen. The reaction mixture was stirred for 3-4 hours. The progress of the reaction was monitored by TLC. The reaction mixture was quenched with water (1×50 mL) slowly (quenching was exothermic). Organic layer was separated and aqueous layer was extracted with ethyl acetate (2×50 mL). The combined organic phase was washed with saturated sodium chloride (1×50 mL). It was dried over anhydrous sodium sulfate, filtered, and the filtrate was concentrated in vacuo to get the crude product 12a as viscous oil. The crude product was purified by column chromatography to yield intermediate 12a, colorless viscous oil (1.0 g, 87%).

### 2.2.2. Synthesis of Compound 75 in FIG. 13

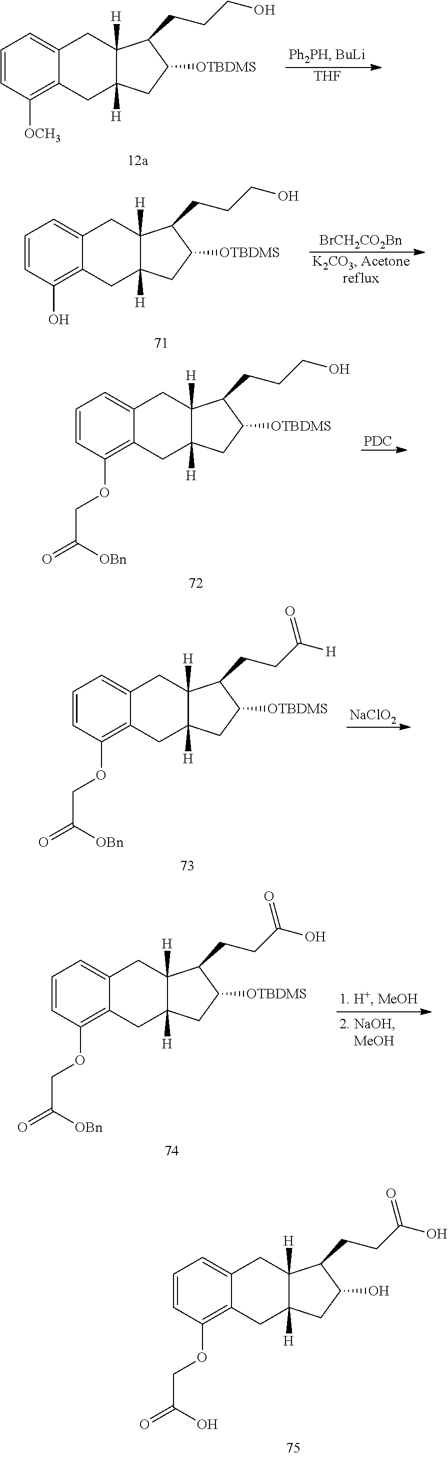

### Experimental

### Synthesis of Intermediate 71

A 100-mL, three-necked, round-bottom flask equipped with a cooling bath, a thermocouple, and an argon inlet-outlet adapter connected to a bubbler was charged with anhydrous tetrahydrofuran (10 mL) and n-butyllithium (1.31 g, 20.45 mmol) under nitrogen. The mixture was cooled to −20 to −30° C. and diphenylphosphine (3.34 g, 17.94 mmol) was added. The resulting orange-reddish solution was stirred for 10-15 minutes. A solution of intermediate 12a (1.00 g, 2.56 mmol) in THF (10 mL) was added to the reaction mixture at −20° C. under nitrogen. After the complete addition, the dark red solution was stirred at −20° C. for 30 minutes, and then the temperature of reaction mixture was allowed to rise to ambient temperature. The reaction mixture was heated at reflux overnight. The progress of the reaction was monitored by TLC. After the completion of the reaction, the reaction mixture was cooled to ambient temperature, and then water was added to quench the reaction. The organic layer was separated, and the aqueous layer was extracted with ethyl acetate (2×50 mL). The combined organic layers are dried over anhydrous sodium sulfate (10 g), filtered and concentrated in vacuo to get crude product 71. The crude product 71 was purified by column chromatography to yield product 71 1.3 g (yield was more than 100% (964 mg), probably due to presence of residual solvents).

### Synthesis of Intermediate 72

A 100-mL, one-neck, round-bottom flask equipped with a magnetic stirrer, an argon inlet-outlet adapter connected to a bubbler and a condenser was charged with intermediate 71 (1.3 g, 3.45 mmol) and acetone. The mixture was stirred to get a clear solution. Benzylbromoacetate (1.186 g, 5.18 mmol), powdered potassium carbonate (1.431 g, 10.36 mmol) were added to the above solution. The reaction mixture was heated to reflux for 3-4 h. The progress of the reaction was monitored by TLC. After the 3.5 hours, the reaction was not complete (TLC), for this reason, additional amount to reagents benzylbromoacetate (0.5 g, 2.18 mmol), potassium carbonate (1.0 g, 7.24 mmol) are added. The reaction mixture was heated again to reflux. After completion of reaction (TLC), the reaction mixture was cooled to ambient temperature, and it was filtered through a pad of Celite. The pad of the Celite was washed with acetone, and the filtrate was concentrated in vacuo to give a viscous liquid. The crude product 72 was purified by column chromatography to yield a viscous liquid of pure product 72, (1.15 g, yield 85% based on 100% yield of previous step)

### Synthesis of Intermediate 73

A 100-mL, one-neck, round-bottom flask equipped with a magnetic stirrer, was charged with intermediate 72 (1.1 g, 2.10 mmol) and dichloromethane. The mixture was stirred to get a clear solution. Celite and sodium acetate (0.35 g, 4.27 mmol) are added to the above solution. After stirring of reaction mixture (5-10 minutes), PDC (1.58 g, 4.20 mmol) was added at ambient temperature. The progress of the reaction was monitored by TLC after 3-4 hours. After the 3.5 hours, the reaction was complete (TLC). The product 73 was purified by column chromatography using 230-400 mesh silica gel by directly loading the reaction mixture on column, and the column was eluted with dichloromethane (100%). The fractions containing the desired compound 73 are evaporated in vacuo to yield a viscous liquid of pure product 73, (0.90 g, yield 83%)

### Synthesis of Intermediate 74

A 50-mL, one-neck, round-bottom flask equipped with a magnetic stirrer, was charged with intermediate 73 (0.82 g, 1.57 mmol) and tert-butanol (15-20 mL) and water. The mixture was stirred to obtain a clear solution. Sodium phosphate monobasic (1.13 g, 9.42 mmol) and 2-methyl-2-butene (2.0 mL) was added. The mixture was stirred for 5-10 minutes. To the resulting suspension, add sodium chlorate (1.30 g, 14.37 mmol) at ambient temperature. The progress of the reaction was monitored by TLC after 2-3 hours. After completion of reaction, the solvent was evaporated in vacuo to give a viscous liquid. Water was added to the residual material, the aqueous layer was acidified to pH 3-4 and extracted with ethyl acetate (3×50 mL). The organic layer was washed with brine, dried over anhydrous sodium sulfate. The mixture was filtered and the filtrate was evaporated in vacuo to give crude product. The product 74 was purified by column chromatography to yield a viscous liquid of pure product 74, (0.740 g, yield 88%).

### Synthesis of Compound 75 in FIG. 13

A 50-mL, one-neck, round-bottom flask equipped with a magnetic stirrer, was charged with a solution of intermediate 74 (1.37 g, 1.37 mmol) in methanol. To the reaction mixture, a solution HCl (1.3 ml, 37%, 13.18 mmol, dissolved in water (5 mL)) was added. The mixture was stirred at room temperature for 6-7 hours. After 6-7 hours, a solution of sodium hydroxide (1.1 g, 27.5 mmol, dissolved water (2 mL)) was added. The reaction mixture was heated to reflux overnight. The progress of the reaction was monitored by TLC. After completion of reaction, the solvent was evaporated in vacuo. Water was added to the residual material, the aqueous layer was extracted with ethyl acetate (3×20 mL) to remove impurities. The extracted ethyl acetate layers were discarded after TLC check. The aqueous layer was acidified to pH (1-2) and extracted with ethyl acetate (3×70 mL). The organic layer was washed with brine, dried over anhydrous sodium sulfate. The mixture was filtered and the filtrate was evaporated to give crude product (425 mg). The crude material triturated with dichloromethane (15 mL) and followed by hexanes (15 mL). The suspension of solid was filtered to isolate solid. The solid was washed with hexanes, dried on funnel and then air-dried in a fume-hood at room temperature to give pure product 75, (385 mg, and yield 84%)

### 2.2.3. Synthesis of Sodium Salt of Compound 108 in FIG. 13

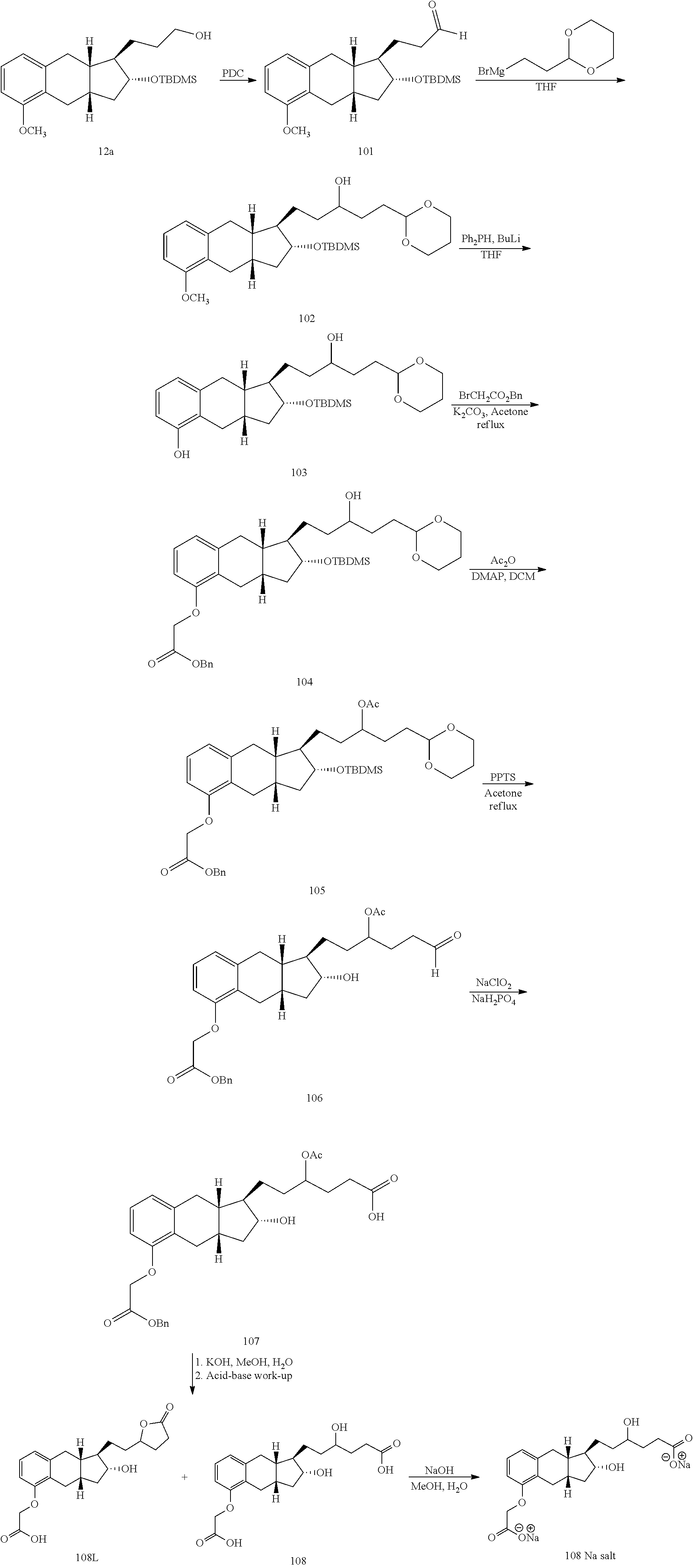

### Experimental

### Synthesis of Intermediate 101

A 100-mL, one-neck, round-bottom flask equipped with a magnetic stirrer, was charged with intermediate 12a (3.60 g, 9.22 mmol) and dichloromethane (60-100 mL). The mixture was stirred to get a clear solution. Celite (7.0 g) and sodium acetate (1.80 g, 21.94 mmol) were added to the above solution. After stirring of reaction mixture (5-10 minutes), PDC (6.9 g, 18.34 mmol) was added at ambient temperature. The progress of the reaction was monitored by TLC after 3-4 hours. After the 3.5 hours, the reaction was complete (TLC). The product 101 was purified by column chromatography using 230-400 mesh silica gel by directly loading the reaction mixture on column, and the column was eluted with dichloromethane (100%). The fractions containing the desired compound 101 are evaporated in vacuo to yield a viscous liquid of pure product 101, (2.846 g, and 80%)

### Synthesis of Intermediate 102

A 250-mL, three-neck, round-bottom flask equipped with a cooling bath, a thermocouple, and an argon inlet-outlet adapter connected to a bubbler was charged with anhydrous tetrahydrofuran (20 mL) and (1,3-dioxan-2-ylethyl)-magnesium bromide solution 0.5M in THF (17.3 mL, 8.65 mmol) under nitrogen. The mixture was cooled to −20° C. and a solution of intermediate 101 in THF (2.8 g, 7.21 mmol, dissolved in dry THF (20 mL)) was charged. The temperature of the reaction mixture was allowed to increase from −20° C. to ambient temperature. The progress of the reaction was monitored by TLC after 3-4 hours. After the 3.5 hours, the reaction was complete (TLC). The reaction mixture was quenched with saturated ammonium chloride solution (2 mL) while stirring. The reaction mixture converted to a suspension of white granular solid. The resulting suspension was filtered and the filtrate was evaporated in vacuo to yield crude product 102. The product 102 was purified by column chromatography using 230-400 mesh silica gel, the column was eluted with gradient of ethyl acetate in hexanes (15-40%). The fractions containing the desired pure compound 102 were combined and evaporated in vacuo to yield a viscous liquid of pure product 102, (3.698 g, quantitative yield)

### Synthesis of Intermediate 103

A 250-mL, three-necked, round-bottom flask equipped with a cooling bath, a thermocouple, and an argon inlet-outlet adapter connected to a bubbler was charged with anhydrous tetrahydrofuran (20 mL) and n-butyllithium (20.3 mL, 50.73 mmol, 2.5 M in hexanes) under nitrogen. The mixture was cooled to −20 to −30° C. and diphenylphosphine (8.30 g, 44.58 mmol) was added. The resulting orange-reddish solution was stirred for 10-15 minutes. A solution of intermediate 102 (3.20 g, 6.34 mmol) in THF (20 mL) was added to the reaction mixture at −20° C. under nitrogen. After the complete addition, the red solution was stirred at −20° C. for 30 minutes, and then the temperature of reaction mixture was allowed to rise to ambient temperature. The reaction mixture was heated to reflux overnight. The progress of the reaction was monitored by TLC. After completion of the reaction, the reaction mixture was cooled to ambient temperature, and then water was added slowly to quench the reaction. The color of reaction was changed from red to colorless. The organic layer was separated, and the aqueous layer was extracted with ethyl acetate (2×100 mL). The combined organic layers were dried over anhydrous sodium sulfate, filtered and concentrated in vacuo to get crude product 103. The crude product 103 was purified by column chromatography to yield product 103, (3.0 g, yield 96%).

### Synthesis of Intermediate 104

A 250-mL, one-neck, round-bottom flask equipped with a magnetic stirrer, an argon inlet-outlet adapter connected to a bubbler and a condenser was charged with intermediate 103 (3.00 g, 6.11 mmol) and acetone (30-50 mL). The mixture was stirred to get a clear solution. Benzylbromoacetate (2.10 g, 9.17 mmol), powdered potassium carbonate (4.23 g, 30.61 mmol) are added to the above solution. The reaction mixture was heated at reflux overnight. The progress of the reaction was monitored by TLC. After completion of reaction (checked by TLC), the reaction mixture was cooled to ambient temperature, and it was filtered through a pad of Celite in a Buchner funnel. The pad of the Celite was washed with acetone, and the combined filtrates are concentrated in vacuo to give a viscous liquid. The crude product 104 was purified by column chromatography to yield a viscous liquid of pure product 104, (3.75 g, and yield 96%)

### Synthesis of Intermediate 105

A 250-mL, one-necked, round-bottom flask equipped with a magnetic stirrer was charged with a solution of crude Intermediate 104 (3.70 g, 5.79 mmol), dichloromethane (50-100 mL), dimethylaminopyridine (DMAP, 1.77 g, 14.49 mmol), and acetic anhydride (1.30 g, 12.73 mmol) at room temperature under argon. The reaction was stirred overnight. After ˜16 h, the progress of the reaction was monitored by TLC. For purification of product 105, the reaction mixture directly loaded on column packed with silica gel. The column was eluted with a gradient solvent of ethyl acetate in hexanes. The fractions containing the desired compound were combined and evaporated in vacuo to yield intermediate 105, colorless viscous oil (3.72 g, 94%).

### Synthesis of Intermediate 106

A 250-mL, one-neck, round-bottom flask equipped with a magnetic stirrer, and a condenser was charged with intermediate 105 (3.70 g, 5.43 mmol) and acetone (100 mL). The mixture was stirred to get a clear solution. Pyridinium p-toluenesulfonate (PPTS) (2.50 g, 9.95 mmol), and water (50 mL) are added to the above solution. The reaction mixture was heated to reflux overnight. The progress of the reaction was monitored by TLC and by ¹H-NMR. Heating of reaction mixture to reflux was continued until completion of reaction was confirmed by ¹H-NMR. After completion of reaction, the reaction mixture was cooled to ambient temperature, the solvent was concentrated in vacuo to give aqueous layer containing product. Additional amount of water was added. The aqueous layer was extracted with dichloromethane (3×50 mL). The organic layer was washed with brine, dried over anhydrous sodium sulfate and filtered. The filtrate was concentrated in vacuo to give crude product 106. The crude product was purified by column chromatography to yield a viscous liquid of pure product 106, (1.693 g, yield 61%)

### Synthesis of Intermediate 107

A 100-mL, one-neck, round-bottom flask equipped with a magnetic stirrer, was charged with intermediate 106 (0.628 g, 1.23 mmol) and tert-butanol (20-30 mL) and water (10-15 mL). The mixture was stirred to obtain a clear two-phase solution. Sodium phosphate monobasic (0.889 g, 7.41 mmol) and 2-methyl-2-butene (5-7 mL) was added. The mixture was stirred for 5-10 minutes. To the resulting suspension, add sodium chlorate (1.117 g, 12.35 mmol) at ambient temperature. The progress of the reaction was monitored by TLC after 2-3 hours. After completion of reaction, the solvent was evaporated in vacuo to give a viscous liquid. Water was added to the residual material, the aqueous layer was acidified to pH 3-4 and extracted with ethyl acetate (3×50 mL). The organic layer was washed with brine, dried over anhydrous sodium sulfate. The mixture was filtered and the filtrate was evaporated to give crude product. The product 107 was purified by column chromatography to yield a viscous liquid of pure product 107, (600 mg, yield 93%)

### Synthesis of Metabolite 108L and 108

A 50-mL, one-neck, round-bottom flask equipped with a magnetic stirrer, was charged with a solution of intermediate 107 (1.1 g, 2.10 mmol) in methanol (15-20 mL). To the reaction mixture, add a solution of KOH (1.18 g, 21.03 mmol, dissolved in water (5 mL)). The reaction mixture was heated to reflux for 2-6 hours. The progress of the reaction was monitored by TLC. After completion of reaction, the solvent was evaporated in vacuo. Water was added to the residual material, the aqueous layer was extracted with dichloromethane (7×50 mL) to remove impurities. The aqueous layer was acidified to pH (1-2) and extracted with ethyl acetate (4×70 mL). The organic layer was washed with brine, dried over anhydrous sodium sulfate. The mixture was filtered and the filtrate was evaporated in vacuo to give crude product (900 mg). As per ¹H NMR and HPLC, the crude material was a mixture of two products viz the lactone of 108L and 108.

### Synthesis of Racemic Metabolite 108Na

A 50-mL, one-neck, round-bottom flask equipped with a magnetic stirrer, was charged with a solution of intermediate 108L (0.750 g, 2.00 mmol) in methanol (10 mL). To the reaction mixture, add a solution NaOH (0.168 g, 4.20 mmol, dissolved in water (2-3 mL)). The reaction mixture was heated to reflux for 3-4 hours. The progress of the reaction was monitored by HPLC. After completion of reaction, the solvent was evaporated in vacuo. The resulting material was dissolved in anhydrous ethanol (5-10 mL) in order to remove water by azeotropic evaporation in vacuo. The gummy material was evaporated to dryness. Mixture of dichloromethane and hexanes (1:1) (40-60 mL) was added to material. The mixture was stirred for 1-2 hours for trituration. The fine solid was isolated by filtration. The solid material was air-dried to give material 108 Na salt (800 mg, yield 92%).

### 2.2.4. Synthesis of Compound 117 In FIG. 13

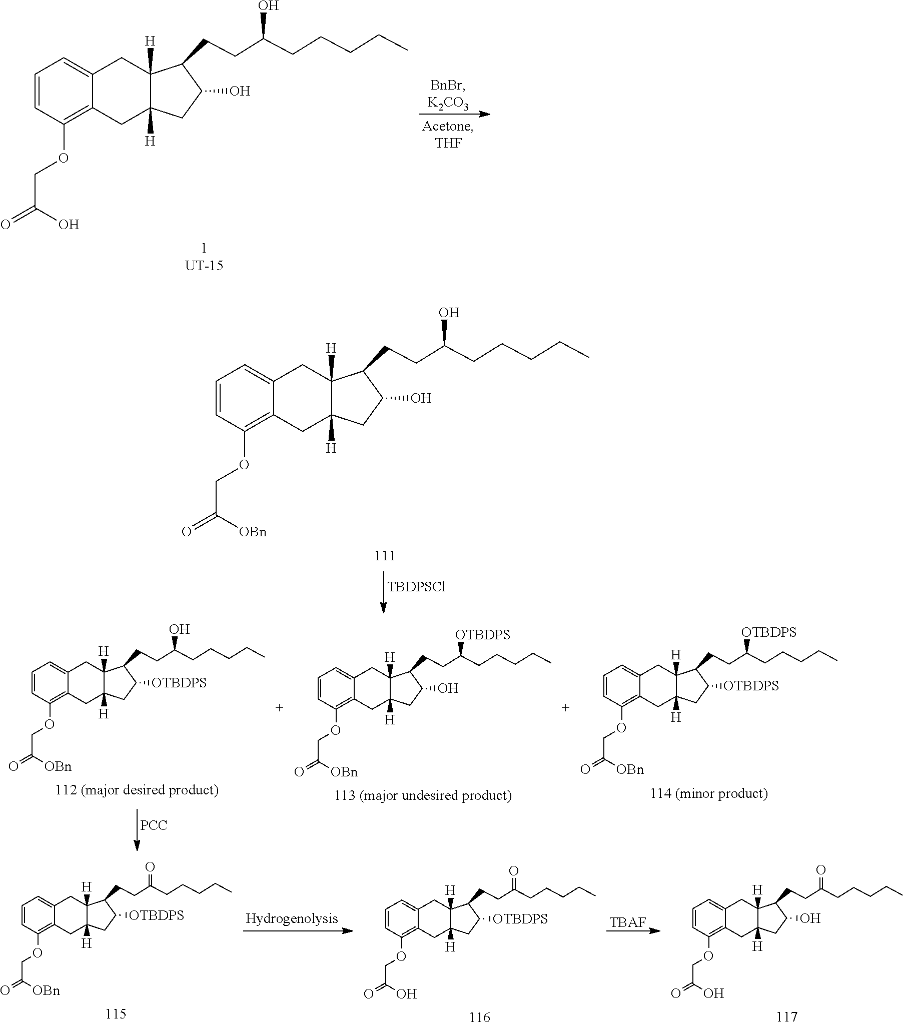

### Experimental

### Synthesis of treprostinil Benzyl ester (111)

A 100 mL, round-bottom flask equipped with a magnetic stirrer, was charged with solution of treprostinil (1) (2 g, 0.0025 mol) in THF:Acetone (1:4, 10:40 mL). To this clear solution potassium carbonate (2.82 g, 0.0204 mol) was added in one portion followed by benzyl bromide (1.75 g, 0.0102 mol) while stirring at room temperature. The reaction mixture was stirred until completion (˜16-18 h) of the reaction and the progress of the reaction was monitored by TLC. The mixture was filtered through a Buchner funnel. The filtrate was removed in vacuo, and the crude product was triturated with hexanes (100 mL) to yield treprostinil benzyl ester (111) as off white solid (2 g, 83%). This was used in the next step without further purification.

### Synthesis of TBDPS ether (112)

A 100 mL, three-necked, round-bottom flask equipped with a magnetic stirrer, a thermocouple, and an argon inlet-outlet trap was charged with a solution of treprostinil benzyl ester (111) (780 mg, 0.0016 mol), dichloromethane (15 mL), imidazole (143 mg 0.0021 mol), and 4-(dimethylamino)pyridine (9 mg, 5 mol %), at room temperature under argon. The mixture was stirred until a clear solution was obtained. The mixture was cooled to 0° C. (ice/water bath) and t-butyldiphenylsilyl chloride (535 mg, 0.0019) was added in portions while keeping the temperature below 20° C. The reaction mixture was stirred overnight. The temperature of the reaction mixture was allowed to rise to ambient temperature. Progress of the reaction was monitored by TLC. After ˜16 h reaction was complete. The reaction mixture was washed with water (10 mL) and saturated sodium chloride (10 mL) followed by drying over anhydrous sodium sulfate, filtered, and concentrated in vacuo to get the crude product 112 as a viscous oil. The crude product was purified by column chromatography to yield tert-butyldiphenylsilyl ether (112) (290 mg, 25%) as a colorless viscous oil.

### Synthesis of Ketone (115)

A 100 mL, round-bottom flask equipped with a magnetic stirrer, was charged with mono protected-TBDPS alcohol (112) (297 mg, 0.0004 mol), dichloromethane (10 mL). To this suspension pyridinium chlorochromate (PCC) (137 mg, 0.0064 mol) was added while stirring. The reaction mixture was stirred until completion (˜16-18 h) and progress of the reaction was monitored by TLC. The mixture was filtered through a pad of Celite using a Buchner funnel. The solvent was removed in vacuo, and the crude product was purified by column chromatography to yield ketone, (115) as a light-yellow, viscous oil (290 mg, 95%).

### Synthesis of TBDPS protected Keto acid (116)

To a solution of tricyclic enone mono protected-TBDPS ketone (115) (7.0 g, 0.0099 mol) in methanol (60 mL) was added 10% Pd/C (600 mg, 50% wet, 25% w/w) and the mixture was hydrogenated at atmospheric pressure (balloon pressure) for 15-18 h at room temperature. The progress of the reaction was monitored by TLC. After the reaction was complete, the reaction mixture was filtered through Celite and washed with ethanol. This ethanolic solution was evaporated in vacuo to obtain a keto acid (116) (7.0 g crude yield) and this material was used in the next step without further purification.

### Synthesis of Compound 117

To a stirred solution of mono protected-TBDPS keto acid (116) (7.0 g, 0.011 mol) in tetrahydrofuran (85 mL) was added a solution of tetrabutylammonium fluoride (30.5 g, 0.117 mol) in THF (15 mL) at room temperature under argon. The reaction was stirred at room temperature until completion of reaction as indicated by TLC. After the reaction was complete the solvent was removed in vacuo to yield a crude brown oil which was purified by acid-base extraction to yield compound 117 as a light brown solid (3.61 g, 80.2%).

### 2.3 Synthesis of Compound 126 In FIG. 13

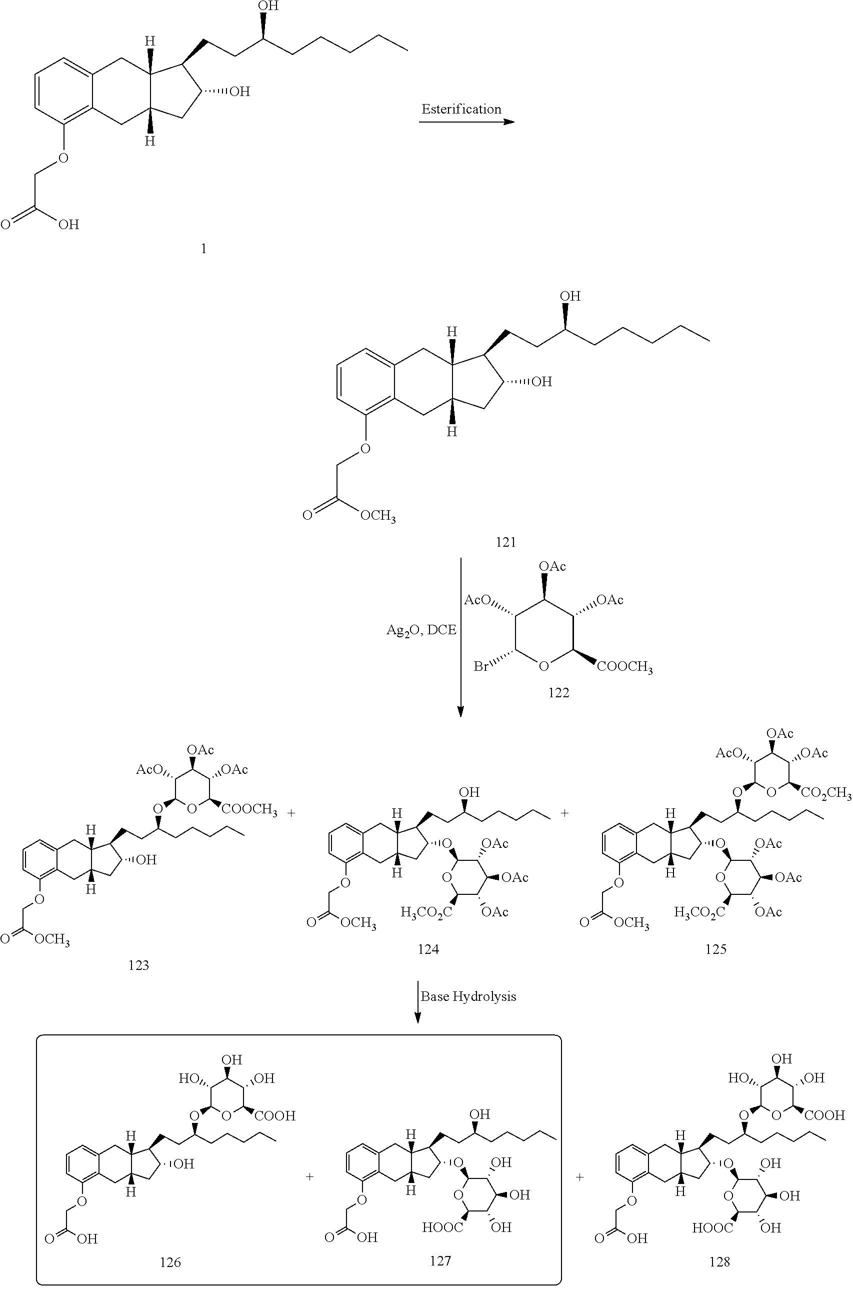

### Experimental

### Synthesis of treprostinil Methyl Ester (121)

A 1000 mL round-bottom flask equipped with a magnetic stirrer, was charged with solution of treprostinil (1) (40 g, 0.110 mol) in methanol (400 mL). To the clear solution catalytic amount of sulfuric acid (4 mL) was added while stirring at room temperature. The reaction mixture was heated to reflux until completion (˜2 h) of the reaction and progress of the reaction was monitored by TLC. The solvent was removed in vacuo, and the crude product was purified by column chromatography to yield treprostinil methyl ester (121) as a viscous oil (46.4 g, 99%).

### Synthesis of Protected Glucuronide (123)

A 100 mL round-bottom flask equipped with a magnetic stirrer, was charged with solution of treprostinil methyl ester (121) (1.0 g, 0.0024 mol) in 1,2-dichloroetahne (30 mL). To the clear solution silver oxide (1.19 g, 0.0051 mol), α-bromotriacetoxymethylester (122) D-glucose and molecular sieve (4 A° type) were added while stirring the mixture at room temperature. The reaction mixture was stirred at room temperature until completion (˜16-18 h) of the reaction and progress of the reaction was monitored by TLC. The reaction mixture was filtered and solvent was removed in vacuo, and the crude product (123) was used as such for the next reaction (3.8 g, crude yield, theoretical yield 1.39 g).

### Synthesis of Compound 126

A 100 mL round-bottom flask equipped with a magnetic stirrer, was charged with solution of protected glucuronide (123) (3.8 g crude from previous step, 0.002 mol, based on 100% yield) in methanol (16 mL). To the clear solution aqueous solution of sodium hydroxide (1.17 g, 0.029 mol, solution in water 8 mL) was added while stirring the mixture at room temperature. The reaction mixture was stirred at room temperature until the hydrolysis was complete (˜16-18 h) and progress of the reaction was monitored by HPLC. The reaction mixture was neutralized to pH 5-7 using 2M HCl (10 mL). The reaction mixture was extracted with ethyl acetate (2×25 mL), ethyl acetate layer containing crude mixture of isomeric glucuronides (126+127) was washed with brine (10 mL), dried over sodium sulfate and the solvent was removed in vacuo to obtain a crude compound 126 (12.6 g, crude). Pure metabolite 126 was collected via prep HPLC.

### 2.4 Synthesis of Compound 96A

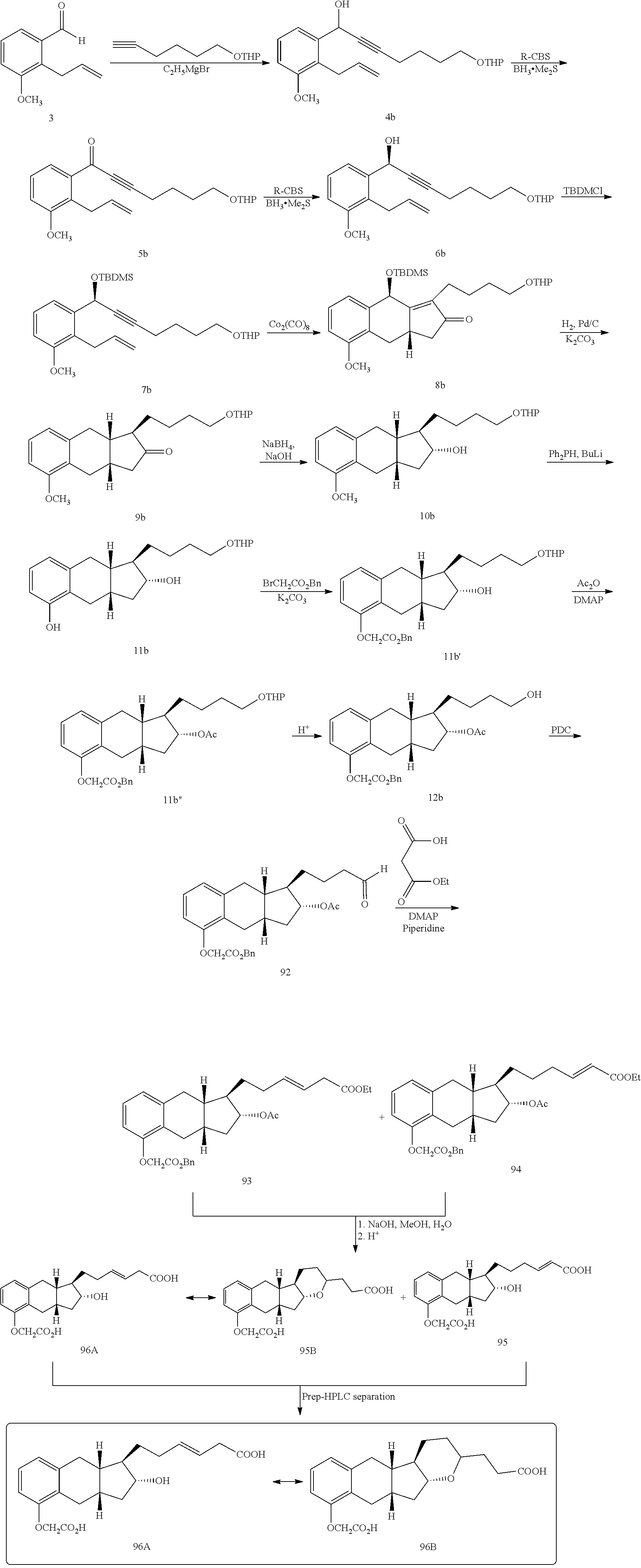

### Experimental

### Synthesis of Intermediate 4b

A 1-L, three-necked, round-bottom flask equipped with a mechanical stirrer, an addition funnel, a thermocouple and an argon inlet-outlet adapter connected to a bubbler was charged with 1-hexyn-O-tetrahydropyran-2-yl-5-ol (26.90 g, 0.148 moles), and anhydrous tetrahydrofuran (300-350 mL). Ethyl magnesium bromide (3M solution in diethyl ether, 49.0 mL, 0.148 moles) was added to the solution under argon keeping the temperature below 40° C. over a period of 20-30 minutes. After complete addition, the reaction mixture was stirred between 30° C. and 45° C. for 2 h. The reaction mixture was cooled to 0° C. (ice-water bath), and a solution of 2-allyl-3-methoxybenzaldehyde (3, 20.0 g, 0.113 moles) in anhydrous tetrahydrofuran (50 mL) was added over a period of 10 minutes. The reaction mixture was stirred overnight at room temperature and the temperature of the reaction mixture was allowed to rise to ambient temperature. After 16 h, the reaction was complete (TLC). The reaction mixture was quenched with saturated ammonium chloride at ambient temperature, which resulted in formation of granular solid. The mixture was filtered to remove granular inorganic solid, and the filtrate was concentrated in vacuo to get a crude product 4b (45 g). The crude product was purified by column chromatography to give pure intermediate 4b as pale-yellow, viscous liquid (36 g).

### Synthesis of Intermediate 5b

A 2-L, two-necked, round-bottom flask equipped with a mechanical stirrer, a thermocouple was charged with intermediate 4b (36.0 g, 0.10 moles), acetone (350-400 mL). At ambient temperature, manganese dioxide (MnO₂) (87.3 g, 1.00 moles) was added while stirring. The reaction mixture was stirred overnight. The mixture was filtered through a pad of Celite using a Buchner funnel. The filtrate was concentrated in vacuo to give an intermediate 5b as a light-yellow, viscous oil (35 g, 68%).

### Synthesis of Intermediate 6b

A 1-L, three-necked, round-bottom flask equipped with a mechanical stirrer, a thermocouple, and an argon inlet-outlet trap was charged with aryl intermediate 5b (35 g, moles 0.098), and anhydrous tetrahydrofuran (300-400 mL). A solution of (R)-methyl oxazaborolidine (117.8 mL, 1.0 M in toluene, moles 0.118) was added under argon at room temperature. The mixture was cooled to −30° C. (dry ice/acetone-bath), and borane-methyl sulfide complex (17 mL, moles 0.196) was added slowly keeping the temperature between −25° C. and −30° C. After complete addition, the reaction mixture was stirred for 1 h at the same temperature. Progress of the reaction was monitored by TLC. The reaction mixture was carefully quenched by slow addition of methanol (30 mL) and followed by addition of saturated ammonium chloride (50 mL). The solid material was removed by filtration. The filtrate was evaporated in vacuo to give a viscous solid. The viscous solid was dissolved in ethyl acetate and filtered again to remove insoluble impurities. The filtrate was evaporated in vacuo to give a viscous solid. The crude product was purified by column chromatography to afford an intermediate 6b (a semi-solid product) (16.1 g).

### Synthesis of Intermediate 7b

A 1-L, three-necked, round-bottom flask equipped with a mechanical stirrer, a thermocouple, and an argon inlet-outlet trap was charged with a solution of crude chiral benzylalkynol (6b) (16.00 g), dichloromethane (160-250 mL), imidazole (4.860 g), and dimethylaminopyridine (0.273 g) at room temperature under argon. The mixture was stirred until a clear solution was obtained and t-butyldimethylsilyl chloride (10.1 g) was added while keeping the temperature below 20° C. The reaction was stirred overnight. The temperature of the reaction mixture was allowed to rise to ambient temperature. After ˜16 h, the progress of the reaction was monitored by TLC. The mixture was washed with water (3×150 mL) and saturated sodium chloride (1×150 mL), dried over anhydrous sodium sulfate (10 g), filtered, and concentrated in vacuo to get the crude product 7b as viscous oil. The crude product was purified by column chromatography to yield benzyl alkynyl-t-butyldimethylsilyl ether (intermediate 7b), colorless viscous oil (16.1 g, 77%).

### Synthesis of Intermediate 8b

A 250 mL, three-necked, round-bottom flask equipped with a mechanical stirrer, a thermocouple, and an argon inlet-outlet trap was charged with a solution of benzyl alkynyl t-butyldimethylsilyl ether (7b, 16.10 g, 0.0341) in toluene (160 mL) under argon. At room temperature, dicobalt octacarbonyl (11.60 g, 0.0341) was added, and the reaction mixture was stirred at ambient temperature. Carbon monoxide evolved slowly from the reaction mixture, and the solution turned reddish-brown after some time. At this stage the solution was heated at reflux under argon for 2 h. The reaction mixture was cooled to room temperature, and air was bubbled through the mixture overnight. After completion of the reaction, the reaction mixture was diluted with saturated ammonium chloride solution and the mixture was extracted with ethyl acetate (2×125 mL). The combined organic layers were washed with brine (200 mL), dried over anhydrous sodium sulfate and filtered. The filtrate was concentrated in vacuo to yield crude product 8b, brown oil. The crude product 8b was purified by column chromatography to yield pure tricyclic enone (8b) as light-brown oil (12.1 g, yield, 71%). The crude material was dissolved in ethanol (500 mL). To the solution, activated carbon (1.2 g) was added. The suspension was heated to reflux and hot solution was filtered through a pad of Celite. The filtrate was used as such in the next step.

### Synthesis of Intermediate 9b

A 1000 mL, three-necked, round-bottom flask equipped with a magnetic stirrer, stir bar and an hydrogen filled balloon, was charged with a solution of intermediate 8b (12.1 g) in absolute ethanol (600 mL, from previous step), anhydrous potassium carbonate (600 mg) and palladium on activated charcoal (1.2 g, 10%, 50% wet). The reaction flask was evacuated by vacuum and vacuum was replaced by hydrogen from attached balloon. This step was repeated three times. The mixture was hydrogenated under balloon pressure for 16 h (overnight) at ambient temperature. The progress of the reaction was monitored by IR. After the reaction was complete (confirmed by IR), the reaction mixture was filtered through a pad of Celite. The filtrate was concentrated in vacuo to obtain ˜200 mL solution of 9b and was carried to the next step for sodium borohydride reduction (theoretical yield 8.9 g from 8b, 100%).

### Synthesis of Intermediate 10b

A 500-mL, three-necked, round-bottom flask equipped with a mechanical stirrer was charged with a solution of tricyclic ketone (9b) in ethanol. The solution was cooled to −4° C. and aqueous sodium hydroxide solution (9.1 g, dissolved in 90 mL of water) was added with stirring over a period of 10-15 minutes. The reaction mixture was stirred for an additional 0.5 h and then sodium borohydride (1.8 g) was added. The stirring was continued at −10° C. for 2 h. The progress of the reaction was monitored by TLC. The reaction mixture was quenched carefully by drop wise addition of saturated ammonium chloride solution (50 mL) until pH 9-10 was obtained. The mixture was allowed to warm to ambient temperature. The undesired solid was removed by filtration, and the filtrate was concentrated in vacuo get the crude product (tricyclic alcohol 10b) as an oil (8.1 g). The crude product was purified by column chromatography to obtain pure tricyclic alcohol 10b (7.1 g, 80.3% over two steps).

### Synthesis of Intermediate 11b

A 500 mL, three-necked, round-bottom flask equipped with a cooling bath, a thermocouple, and an argon inlet-outlet adapter connected to a bubbler was charged with anhydrous tetrahydrofuran (100 mL) and n-butyllithium under nitrogen. The mixture was cooled to −20 to −30° C. and diphenylphosphine was added. The resulting orange-reddish solution was stirred for 10-15 minutes. A solution of intermediate 10b in THF (100 mL) was added to the reaction mixture at −20° C. under nitrogen. After the complete addition, the dark red solution was stirred at −20° C. for 30 minutes, and then the temperature of reaction mixture was allowed to rise to ambient temperature. The reaction mixture was heated at reflux overnight. The progress of the reaction was monitored by TLC. After the completion of the reaction, the reaction mixture was cooled to ambient temperature, and then water was added to quench the reaction. The organic layer was separated, dried over anhydrous sodium sulfate, filtered and concentrated in vacuo to get a crude product 11b. The crude product 11b was purified by column chromatography to yield compound 11b (6.61 g, 96%).

### Synthesis of Intermediate 11b′

A 250 mL, one-neck, round-bottom flask equipped with a magnetic stirrer, an argon inlet-outlet adapter connected to a bubbler and a condenser was charged with solution of intermediate 11b (6.61 g, 0.183) in acetone (100 mL). Benzylbromoacetate (5.41 g, 0.023) and powdered potassium carbonate (10.8 g, 0.079) were added to the above solution. The reaction mixture was heated to reflux for 3-4 h. The progress of the reaction was monitored by TLC. After completion of reaction, the reaction mixture was cooled to ambient temperature, and filtered through a pad of Celite. The pad of the Celite was washed with acetone, and the filtrate was concentrated in vacuo to give a viscous liquid. The crude product 11b′ was purified by column chromatography to yield a viscous liquid of pure compound 11b′, (7.7 g, 82.6%).

### Synthesis of Intermediate 11b″

A 250 mL, three-necked, round-bottom flask equipped with a magnetic stirrer, a thermocouple, and an argon inlet-outlet trap was charged with a solution of intermediate 11b′ (7.7 g, 0.015 mol) and dichloromethane (110 mL). To the clear solution 4-(dimethylamino) pyridine (2.2 g, 0.018 mol), and acetic anhydride (1.84 g, 0.0180 mole) were added at room temperature under argon. The mixture was stirred at room temperature overnight. Progress of the reaction was monitored by TLC. After the reaction was complete, the mixture was washed with saturated ammonium chloride (20 mL) organic layer was separated, dried over anhydrous sodium sulfate, filtered, and concentrated in vacuo to get the crude product 11b″ as a viscous oil. The crude product was purified by column chromatography to yield acetate ester (11b″) as a colorless viscous oil (7.8 g, 92.8%)

### Synthesis of Intermediate 12b

A 100-mL, one-necked, round-bottom flask equipped with a magnetic stirrer was charged with a solution of crude intermediate 11b″ (7.6 g, 0.014 mol), diethyl ether (110 mL), and magnesium bromide (2.59 g, 0.140) at room temperature under nitrogen. The reaction mixture was stirred for 3-4 hours. The progress of the reaction was monitored by TLC. The reaction mixture was quenched with water (50 mL). The organic layer was separated and aqueous layer was extracted with ethyl acetate (2×100 mL). The combined organic layers were washed with saturated sodium chloride (50 mL), dried over anhydrous sodium sulfate, filtered, and concentrated in vacuo to get the crude product 12b as a viscous oil. The crude product was purified by column chromatography to yield intermediate 12b, colorless viscous oil (2.06 g, 30%).

### Synthesis of Intermediate 92

A 100 mL, one-neck, round-bottom flask equipped with a magnetic stirrer, was charged with the intermediate 12b and dichloromethane (50 mL). The mixture was stirred to get a clear solution. To the clear solution, molecular sieves (500 mg) and PDC (2.9 g, 1.75 mol) were added and the reaction mixture was stirred at ambient temperature. The progress of the reaction was monitored by TLC. After 16 h, the reaction was complete. The product was purified by column chromatography to yield a viscous liquid of pure product 92, (1.14 g, yield 55.6%).

### Synthesis of Intermediates 96A and 96B (mixture)

A 100 mL, one-neck, round-bottom flask equipped with a magnetic stirrer, was charged with intermediate 92 (250 mg, 0.0005 mol) and DMF (8 mL). The mixture was stirred to get a clear solution. To the clear solution mono-ethylmalonate (105 mg, 0.0008 mol), DMAP (22 mg, 0.00018), piperidene (9 mg) and acetic acid (8 mg) were added at 5-10° C. and reaction mixture was stirred at ambient temperature. The progress of the reaction was monitored by TLC. After the 1 h, the reaction was complete. At this stage, the reaction mixture was quenched by the addition of saturated ammonium chloride solution (20 mL) and was stirred for five minutes. The organic layer was separated, dried over sodium sulfate, filtered and evaporated in vacuo to obtain a crude mixture of compound 93 and 94 (270 mg). This crude mixture was dissolved in MeOH (8 mL) and to this clear solution aqueous sodium hydroxide (116 mg in 3 mL water) was added at room temperature. This mixture was stirred at room temperature for 16 h and the progress of the reaction was monitored by HPLC. Once the reaction was complete the pH of reaction mixture was adjusted to 1-2 and extracted with ethyl acetate (2×30 mL). The organic layer was washed with brine, dried over anhydrous sodium sulfate, filtered and the filtrate was evaporated in vacuo to give a crude product. The crude product was purified by preparative HPLC to obtain a compound 96A and 96B (˜100 mg).

### 3. Syntheses of Treprostinil Prodrugs

### 3.1. Synthesis of Treprostinil Side Chain Acetate Cyclopentyl Methyl Carbonate

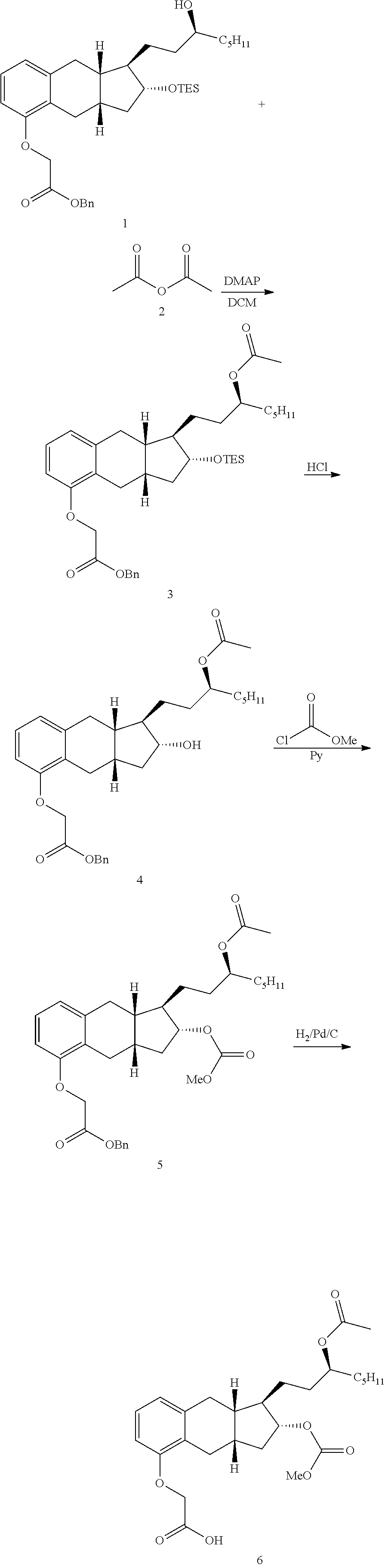

### Experimental

### Synthesis of Mono-TES Treprostinil Benzylester Side Chain Acetate (3)

To a stirring solution of mono-TES treprostinil benzyl ester (1) (3.30 g, 5.55 mmol), DMAP (1.36 g, 11.10 mmol) in dichloromethane (DCM) at room temperature under argon was added acetic anhydride (2) (787 μL, 8.33 mmol). The reaction mixture was stirred at room temperature for 1 h and checked by TLC. The mixture was concentrated in vacuo to give crude product (5.28 g) which was purified by column chromatography to give mono-TES treprostinil benzyl ester side chain acetate (3) (3.47 g, 98% yield) (99.95% HPLC purity). The compound 3 was characterized by ¹H NMR and MS.

### Synthesis of Treprostinil Benzyl Ester Side Chain Acetate (4)

To a stirring solution of mono-TES treprostinil benzyl ester side chain acetate (3) (3.39 g, 5.33 mmol) in tetrahydrofuran (THF) (80 mL) and water (16 mL) at room temperature was added HCl (2N) (2.67 mL, 5.34 mmol). The reaction mixture stirred at room temperature for 1 h and checked by TLC. Water (50 mL) and ethyl acetate (50 mL) were added, and layers were separated. The aqueous layer was extracted with ethyl acetate (2×20 mL) and the combined organic layers washed with brine, dried over sodium sulfate. It was filtrated and the filtrate was concentrated in vacuo to give crude product (3.53 g) which was purified by column chromatography to give treprostinil benzyl ester side chain acetate (4) (2.76 g, 99% yield) (99.82% HPLC purity). The compound 4 was characterized by ¹H NMR and MS.

### Synthesis of Treprostinil Benzyl Ester Side Chain Acetate Cyclopentyl Methyl Carbonate (5)

To a stirring solution of treprostinil benzyl ester side chain acetate (4) (0.72 g, 1.38 mmol) in DCM (10 mL) and pyridine (5 mL) at 0° C. under argon was added methyl chloroformate (0.39 g, 4.14 mmol). The reaction mixture was stirred at room temperature for 2 h and checked by TLC and the reaction was complete. The reaction mixture was concentrated in vacuo to give crude product (1.38 g) which was purified by column chromatography to give treprostinil benzyl ester side chain acetate cyclopentyl methyl carbonate (5) (0.77 g, 96% yield) (99.90% HPLC purity). The compound 5 was characterized by ¹H NMR and MS.

### Synthesis of Treprostinil Side Chain Acetate Cyclopentyl Methyl Carbonate (6)

To a stirring solution of treprostinil benzyl ester side chain acetate cyclopentyl methyl carbonate (5) (0.70 g, 1.20 mmol) in ethyl acetate (20 mL) and water (1 mL) was added palladium on carbon (5 wt. %, 50% water) (100 mg). The reaction mixture was evacuated under house vacuum, filled with hydrogen (repeat this 2 times), connected to hydrogen balloon and stirred at room temperature for 2 h. It was checked by TLC and the reaction was complete. The mixture was filtered through a Celite pad and washed by ethyl acetate. The filtrate was concentrated in vacuo to produce treprostinil side chain acetate cyclopentyl methyl carbonate (6) (0.58 g, 98% yield) (99.73% HPLC purity). The compound 6 was characterized by ¹H NMR, ¹³C NMR, IR and MS.

### 3.2 Synthesis of Treprostinil Side Chain Phosphate Cyclopentyl Propionate

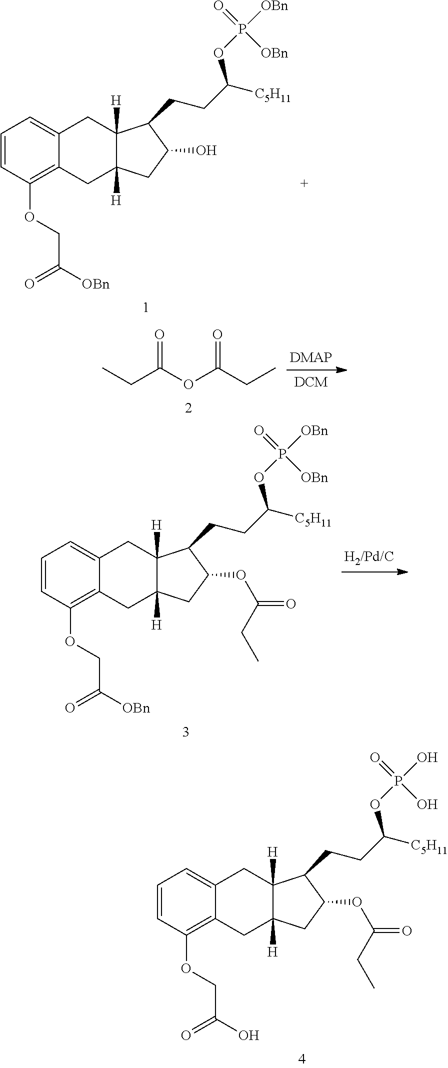

### Experimental

### Synthesis of Treprostinil Benzyl Ester Side Chain Dibenzylphosphate Cyclopentyl Propionate (3)

To a stirring solution of treprostinil benzyl ester side chain dibenzylphosphate (1) (0.99 g, 1.33 mmol) in DCM (20 mL) and DMAP (325 mg, 2.66 mmol) at room temperature under argon was added propionic anhydride (2) (256 μL, 2.00 mmol). The reaction mixture was stirred at room temperature for 1 h. It was checked by TLC and the reaction was complete. The reaction mixture was concentrated in vacuo to give crude product (1.91 g) which was purified by column chromatography to give treprostinil benzyl ester side chain dibenzylphosphate cyclopentyl propionate (3) (1.02 g, 96% yield) (99.11% HPLC purity). The compound 3 was characterized by ¹H NMR and MS.

### Synthesis of Treprostinil Side Chain Phosphate Cyclopentyl Propionate (4)

To a stirring solution of treprostinil benzyl ester side chain dibenzylphosphate cyclopentyl propionate (3) (0.94 g, 1.17 mmol) in ethyl acetate (20 mL) and water (1 mL) was added palladium on carbon (5 wt. %, 50% water) (200 mg). The reaction mixture was evacuated under house vacuum, filled with hydrogen (repeat this 2 times), then connected to hydrogen balloon and stirred at room temperature for 2 hours. It was checked by TLC, the reaction was complete, the mixture was filtered through a Celite pad and washed by ethyl acetate. The filtrate was concentrated in vacuo to produce treprostinil side chain phosphate cyclopentyl propionate (4) (0.60 g, 97% yield) (95.94% HPLC purity). The compound 4 was characterized by ¹H NMR, 13C NMR, ³¹P NMR, IR and MS.

### 3.3 Synthesis of Treprostinil Side Chain Phosphate Cyclopentyl Methyl Carbonate

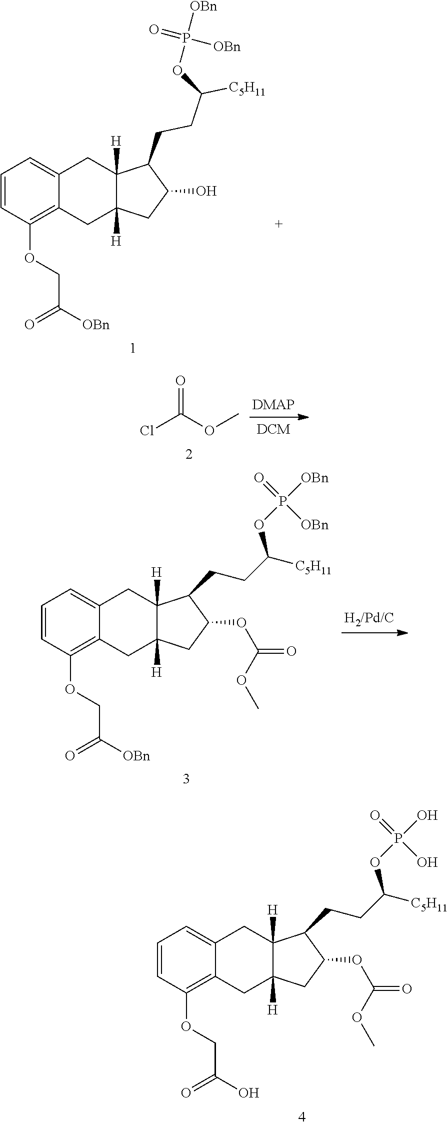

### Experimental

### Synthesis of Treprostinil Benzyl Ester Side Chain Dibenzylphosphate Cyclopentyl Methyl Carbonate (3)

To a stirring solution of treprostinil benzyl ester side chain dibenzylphosphate (1) (1.03 g, 1.38 mmol) in DCM (20 mL) and DMAP (675 mg, 5.52 mmol) at room temperature under argon was added methyl chloroformate (2) (260 mg, 2.76 mmol). The reaction mixture was stirred at room temperature for 2 h. It was checked by TLC and the reaction was complete. The reaction mixture was concentrated in vacuo to give crude product (2.20 g) which was purified by column chromatography to give treprostinil benzyl ester side chain dibenzylphosphate cyclopentyl methyl carbonate (3) (1.04 g, 95% yield) (99.96% HPLC purity). The compound 3 was characterized by ¹H NMR and MS.

### Synthesis of Treprostinil Side Chain Phosphate Cyclopentyl Methyl Carbonate (4)

To a stirring solution of treprostinil benzyl ester side chain dibenzylphosphate cyclopentyl methyl carbonate (3) (0.93 g, 1.16 mmol) in ethyl acetate (20 mL) and water (1 mL) was added palladium on carbon (5 wt. %, 50% water) (250 mg). The reaction mixture was evacuated under house vacuum, filled with hydrogen (repeat this 2 times), connected to hydrogen balloon and stirred at room temperature for 3 h. It was checked by TLC and the reaction was complete. The mixture was filtered through a Celite pad and washed by ethyl acetate. The filtrate was concentrated in vacuo to produce treprostinil side chain phosphate cyclopentyl methyl carbonate (4) (0.62 g, 98% yield) (99.48% HPLC purity). The compound 4 was characterized by ¹H NMR, ¹³C NMR, ³¹P NMR, IR and MS.

### 3.4. Synthesis of Treprostinil Hydroxyacetic Ester Acid Diacetate

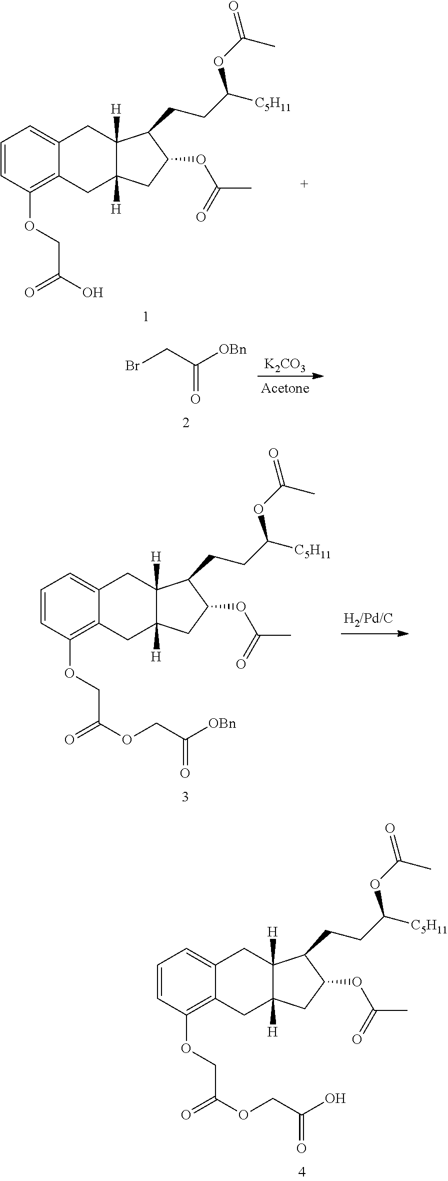

### Experimental

### Synthesis of Treprostinil Hydroxyacetic Benzyl Ester Diacetate (3)

To a stirring solution of treprostinil diacetate (1) (0.20 g, 0.43 mmol) in acetone (2 mL) and potassium carbonate (120 mg, 0.86 mmol) at room temperature was added benzyl bromoacetate (2) (150 mg, 0.65 mmol). The reaction mixture was stirred at room temperature for 2 h and checked by TLC and the reaction was complete. The reaction mixture was filtered and the filtrate was concentrated in vacuo to give crude product (0.57 g) which was purified by column chromatography to give treprostinil hydroxyacetic benzyl ester diacetate (3) (0.23 g, 86% yield) (99.99% HPLC purity). The compound 3 was characterized by ¹H NMR and MS.

### Synthesis of Treprostinil Hydroxyacetic Ester Acid Diacetate (4)

To a stirring solution of treprostinil benzyl hydroxyacetic ester diacetate (3) (0.22 g, 0.35 mmol) in ethyl acetate (10 mL) was added palladium on carbon (5 wt. %, 50% water) (50 mg). The reaction mixture was evacuated under house vacuum, filled with hydrogen (repeat this 2 times), connected to hydrogen balloon and stirred at room temperature for 1 h. It was checked by TLC and the reaction was complete. The mixture was filtered through a Celite pad and washed by ethyl acetate. The filtrate was concentrated in vacuo to produce treprostinil hydroxyacetic ester acid diacetate (4) (0.18 g, 99% yield) (99.78% HPLC purity). The compound 4 was characterized by ¹H NMR, ¹³C NMR, IR and MS.

### 3.5. Synthesis of Treprostinil Hydroxyacetic Ester Acid Dipropionate

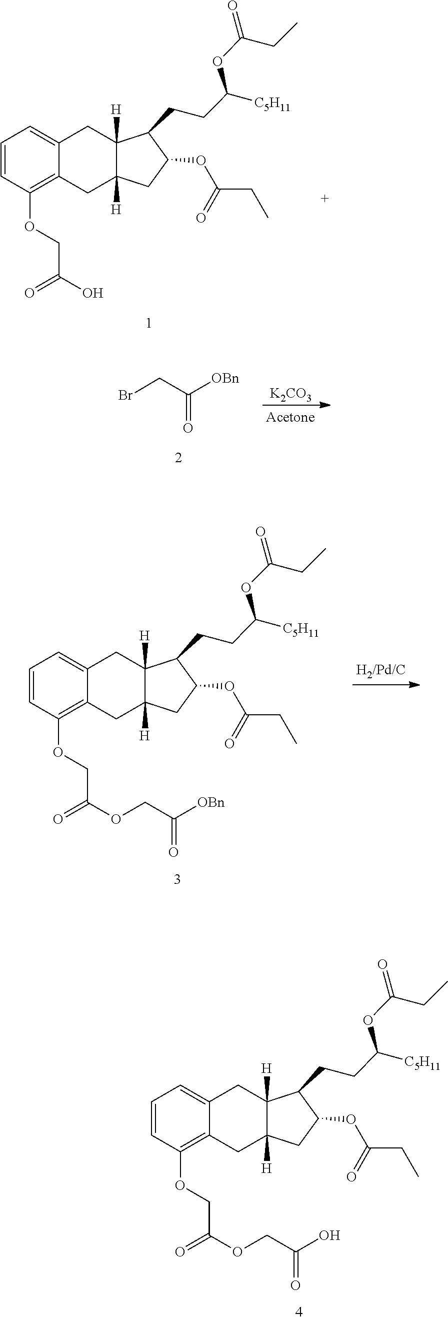

### Experimental

### Synthesis of Treprostinil Hydroxyacetic Benzyl Ester Dipropionate (3)

To a stirring solution of treprostinil dipropionate (1) (0.46 g, 0.91 mmol) in acetone (10 mL) and potassium carbonate (0.25 g, 1.82 mmol) at room temperature was added benzyl bromoacetate (2) (0.31 g, 1.36 mmol). The reaction mixture was stirred at room temperature for 4 h. It was checked by TLC and the reaction was complete. The mixture was filtered, concentrated the filtrate in vacuo to give crude product (0.72 g) which was purified by column chromatography to give treprostinil hydroxyacetic benzyl ester dipropionate (3) (0.57 g, 97% yield) (99.06% HPLC purity). The compound 3 was characterized by ¹H NMR and MS.

### Synthesis of Treprostinil Hydroxyacetic Ester Acid Dipropionate (4)

To a stirring solution of treprostinil hydroxyacetic benzyl ester dipropionate (3) (0.52 g, 0.80 mmol) in ethyl acetate (15 mL) and water (1 mL) was added palladium on carbon (5 wt. %, 50% water) (100 mg). The reaction mixture was evacuated under house vacuum, filled with hydrogen (repeat this 2 times), connected to hydrogen balloon and stirred at room temperature for 1 h. It was checked by TLC and the reaction was complete. The mixture was filtered through a Celite pad and washed by ethyl acetate. The filtrate was concentrated in vacuo to produce treprostinil hydroxyacetic ester acid dipropionate (4) (0.45 g, 99% yield) (99.99% HPLC purity). The compound 4 was characterized by ¹H NMR, ¹³C NMR, IR and MS.

### 3.6. Synthesis of Treprostinil Side Chain 2-((1,3-Bisbenzyloxy)-Propane-2-yloxy) Acetate

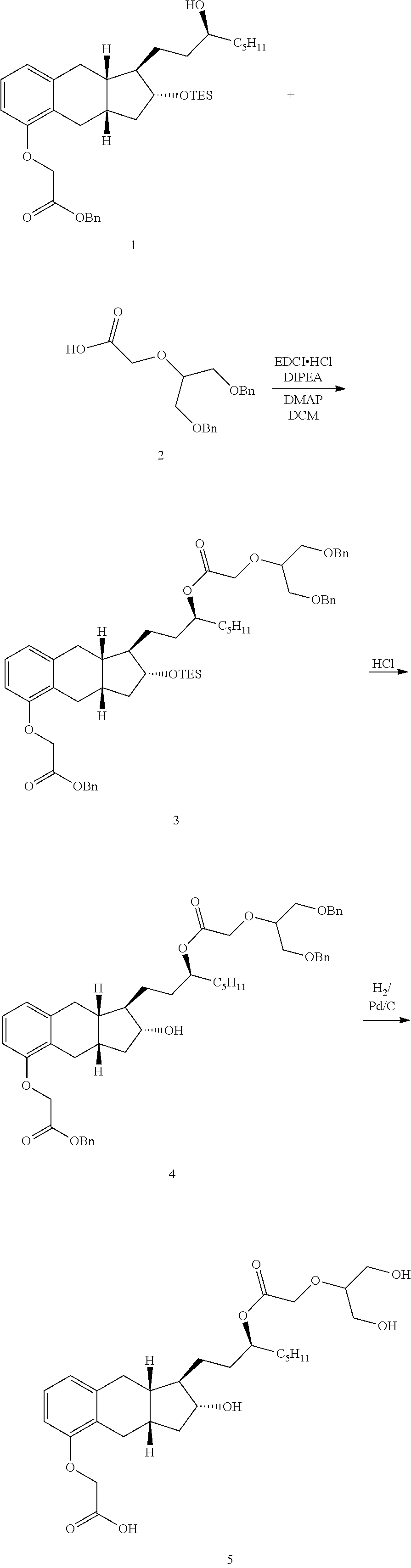

### Experimental

### Synthesis of Mono-TES Treprostinil Benzyl Ester Side Chain 2-((1,3-Bis(benzyloxy)propane-2-yl)oxy) Acetate (3)

To a stirring solution of mono-TES treprostinil benzyl ester (1) (1.55 g, 2.61 mmol), 2-((1,3-bis(benzyloxy)propan-2-yl)oxy)acetic acid (2) (0.72 g, 2.18 mmol), DIPEA (970 μL, 5.45 mmol) and DMAP (53 mg, 0.44 mmol) in dichloromethane (DCM) (25 mL) at room temperature under argon was added EDCl·HCl (1.05 g, 5.45 mmol). The reaction mixture was stirred at room temperature overnight and checked by TLC. Water (20 mL) was added and layers were separated. The aqueous layer was extracted with DCM (2×10 mL). The combined DCM layers were washed with brine, dried over sodium sulfate, filtered, concentrated the filtrate in vacuo to give crude product (2.69 g) which was purified by column chromatography to give mono-TES treprostinil benzyl ester side chain 2-((1,3-bis(benzyloxy)propane-2-yl)oxy) acetate (3) (1.98 g) (84.38% HPLC purity). The compound 3 was characterized by ¹H NMR and MS.

### Synthesis of Treprostinil Benzyl Ester Side Chain 2-((1,3-bis(benzyloxy)propan-2-yl)oxy) Acetate (4)

To a stirring solution of mono-TES treprostinil benzyl ester side chain 2-((1,3-bis(benzyloxy)propane-2-yl)oxy)acetic ester (3) (1.91 g, 2.11 mmol) in tetrahydrofuran (THF) (50 mL) and water (10 mL) at room temperature was added HCl (2N) (2.11 mL, 4.22 mmol). The reaction mixture stirred at room temperature for 1 h. It was checked by TLC and the reaction was complete. Water (30 mL) and ethyl acetate (50 mL) were added and layers were separated. The aqueous layer was extracted with ethyl acetate (2×20 mL). The combined organic layers were washed with brine, dried over sodium sulfate. It was filtrated and the filtrate was concentrated in vacuo to give crude product (2.20 g) which was purified by column chromatography to give treprostinil benzyl ester side chain 2-((1,3-bis(benzyloxy)propan-2-yl)oxy) acetate (4) (1.41 g, 82% yield in 2 steps) (99.09% HPLC purity). The compound 4 was characterized by ¹H NMR and MS.

### Synthesis of Treprostinil Side Chain 2-((1,3-bishydroxy)propane-2-yl)oxy) Acetate (5)

To a stirring solution of treprostinil benzyl ester side chain 2-((1,3-bis(benzyloxy)propane-2-yl)oxy)acetic ester (4) (1.13 g, 1.42 mmol) in ethyl acetate (25 mL) and water (1.5 mL) was added palladium on carbon (5 wt. %, 50% water) (350 mg). The reaction mixture was evacuated under house vacuum, filled with hydrogen (repeat this 2 times), connected to hydrogen balloon and stirred at room temperature for 5 h. It was checked by TLC and ¹H NMR. The reaction was complete. The mixture was filtered through a Celite pad and washed by ethyl acetate. The filtrate was concentrated in vacuo to produce treprostinil side chain 2-((1,3-bishydroxy)propane-2-yl)oxy) acetate (5) (0.65 g, 88% yield) (89.62% HPLC purity). The compound 5 was characterized by ¹H NMR, ¹³C NMR, IR and MS.

### 3.7. Synthesis of Treprostinil Side Chain Phosphate Cyclopentyl Trifluoroacetate

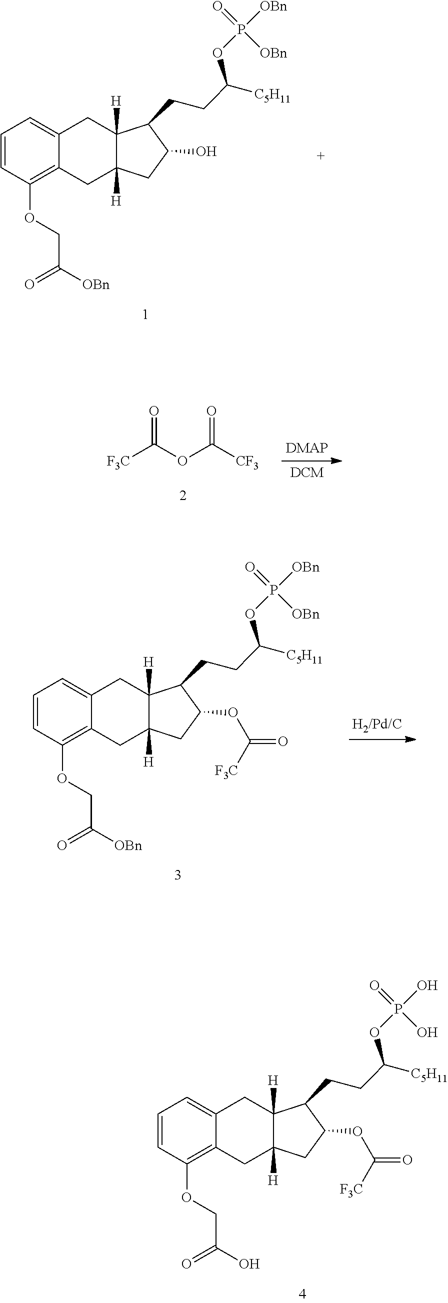

### Experimental

### Synthesis of Treprostinil Benzyl Ester Side Chain Dibenzylphosphate Cyclopentyl Trifluoroacetate (3)

To a stirring solution of treprostinil benzyl ester side chain dibenzylphosphate (1) (0.74 g, 1.00 mmol) in DCM (15 mL) and DMAP (270 mg, 2.20 mmol) at room temperature was added trifluoroacetic anhydride (2) (280 μL, 2.00 mmol). The reaction mixture stirred at room temperature for 1 h and checked by TLC and reaction was complete. The reaction mixture was concentrated in vacuo to give crude product (1.60 g) which was purified by silica gel (˜35 g) column chromatography to give treprostinil benzyl ester side chain dibenzylphosphate cyclopentyl trifluoroacetate (3) (0.55 g, 65% yield) (92.42% HPLC purity). The compound 3 was characterized by ¹H NMR and MS.

### Synthesis of Treprostinil Side Chain Phosphate Cyclopentyl Trifluoroacetate (4)

To a stirring solution of treprostinil benzyl ester side chain dibenzylphosphate cyclopentyl trifluoroacetate (3) (0.54 g, 0.64 mmol) in tetrahydrofuran (THF) (15 mL) and water (1 mL) was added palladium on carbon (160 mg). The reaction mixture was evacuated under house vacuum, filled with hydrogen (repeat this 2 times), connect to hydrogen balloon and stirred at room temperature for 5 h. It was checked by TLC and ¹H NMR and the reaction was complete. The mixture was filtered through a Celite pad and washed by THF. The filtrate was concentrated in vacuo to produce treprostinil side chain phosphate cyclopentyl trifluoroacetate (4) (0.37 g, 99% yield).

### 3.8. Synthesis of Treprostinil Side Chain Triflouroacetate Cyclopentyl Phosphate

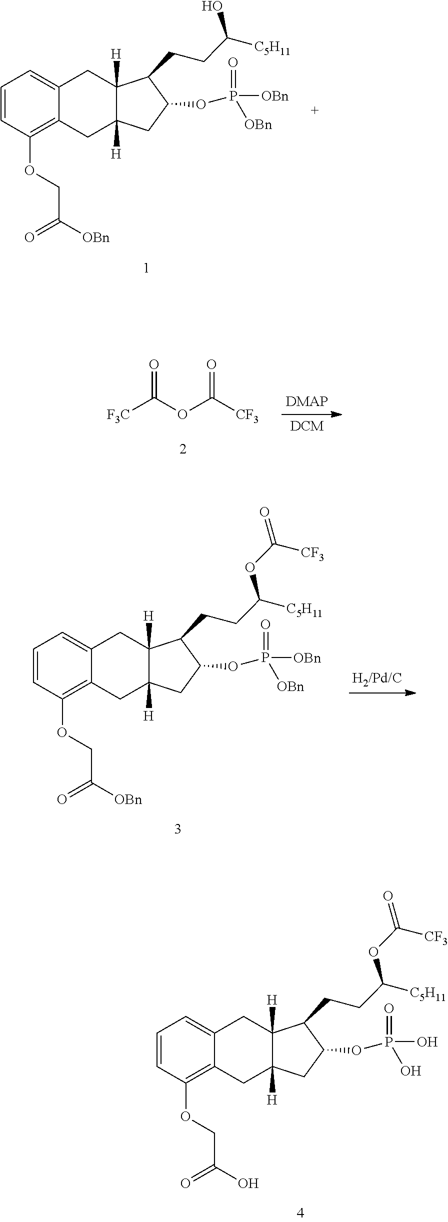

### Experimental

### Synthesis of Treprostinil Benzyl Ester Side Chain Trifluoroacetate Cyclopentyl Dibenzylphosphate (3)

To a stirring solution of treprostinil benzyl ester cyclopentyl dibenzylphosphate (1) (1.07 g, 1.44 mmol) in DCM (20 mL) and DMAP (390 mg, 3.17 mmol) at room temperature was added trifluoroacetic anhydride (2) (400 μL, 2.88 mmol). The reaction mixture was stirred at room temperature for 1 h. It was checked by TLC and reaction was complete. The reaction mixture was concentrated in vacuo to give crude product (2.39 g) which was purified by column chromatography to give treprostinil benzyl ester side chain trifluoroacetate cyclopentyl dibenzylphosphate (3) (0.55 g, 65% yield) (98.64% HPLC purity). The compound 3 was characterized by ¹H NMR and MS.

### Synthesis of Treprostinil Side Chain Trifluoroacetate Cyclopentyl Phosphate (4)

To a stirring solution of treprostinil benzyl ester side chain trifluoroacetate cyclopentyl dibenzylphosphate (3) (0.99 g, 1.18 mmol) in tetrahydrofuran (THF) (20 mL) and water (1 mL) was added palladium on carbon (5 wt. %, 50% water) (300 mg). The reaction mixture was evacuated under house vacuum, filled with hydrogen (repeat this 2 times), connected to hydrogen balloon and stirred at room temperature for 4 h. It was checked by TLC and ¹H NMR and the reaction was complete. The mixture was filtered through a Celite pad and washed with THF. The filtrate was concentrated in vacuo to produce treprostinil side chain trifluoroacetate cyclopentyl phosphate (4) (0.68 g, 99% yield).

### 3.9. Synthesis of Treprostinil Side Chain Trifluoroacetate

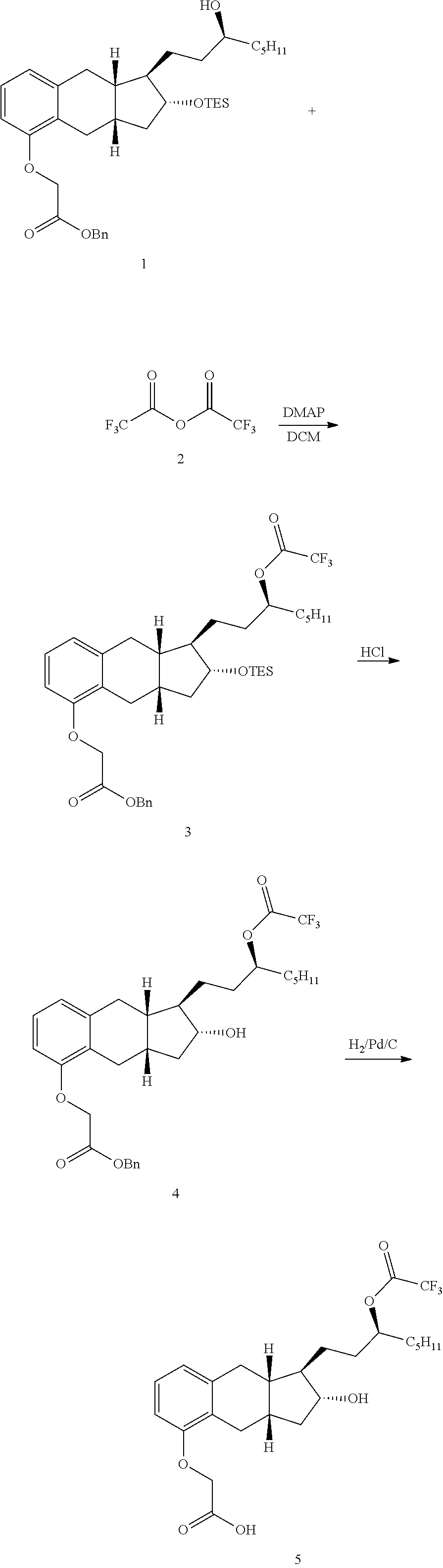

### Experimental

### Synthesis of Mono-TES Treprostinil Benzyl Ester Side Chain Trifluoroacetate (3)

To a stirring solution of mono-TES treprostinil benzyl ester (1) (1.16 g, 1.95 mmol) in DCM (20 mL) and DMAP (596 mg, 4.88 mmol) at room temperature was added trifluoroacetic anhydride (2) (540 μL, 3.90 mmol). The reaction mixture was stirred at room temperature for 1 h. It was checked by TLC and reaction was complete. The reaction mixture was concentrated in vacuo to give crude product (1.91 g) which was purified by column chromatography to give mono-TES treprostinil benzyl ester side chain trifluoroacetate (3) (1.31 g, 96% yield) (99.30% HPLC purity). The compound 3 was characterized by ¹H NMR and MS.

### Synthesis of Treprostinil Benzyl Ester Side Chain Trifluoroacetate (4)

To a stirring solution of mono-TES treprostinil benzyl ester side chain trifluoroacetate (3) (1.22 g, 1.77 mmol) in tetrahydrofuran (THF) (25 mL) and water (5 mL) at room temperature was added HCl (2N) (0.89 mL, 1.78 mmol). The reaction mixture was stirred at room temperature for 1 h. It was checked by TLC and the reaction was complete. Water (20 mL) and ethyl acetate (20 mL) were added and layers were separated. The aqueous layer was extracted with ethyl acetate (2×10 mL). The combined organic layers were washed with brine and dried over sodium sulfate. It was filtrated and the filtrate was concentrated in vacuo to give crude product (1.29 g) which was purified by column chromatography to give treprostinil benzyl ester side chain trifluoroacetate (4) (1.04 g, 99% yield) (99.40% HPLC purity). The compound 4 was characterized by ¹H NMR and MS.

### Synthesis of Treprostinil Side Chain Trifluoroacetate (5)

To a stirring solution of treprostinil benzyl ester side chain trifluoroacetate (4) (0.99 g, 1.72 mmol) in ethyl acetate (20 mL) was added palladium on carbon (5 wt. %, 50% water) (100 mg). The reaction mixture was evacuated under house vacuum, filled with hydrogen (repeat this 2 times), connected to hydrogen balloon, stirred at room temperature for 1 h. It was checked by TLC and the reaction was complete. The mixture was filtered through a Celite pad and washed by ethyl acetate. The filtrate was concentrated in vacuo to produce treprostinil side chain trifluoroacetate (5) (0.95 g, 99% yield) (97.68% HPLC purity). Compound 5 was characterized by ¹H NMR, ¹³C NMR, IR and MS.

### 3.10. Synthesis of Treprostinil Side Chain Diflouroacetate

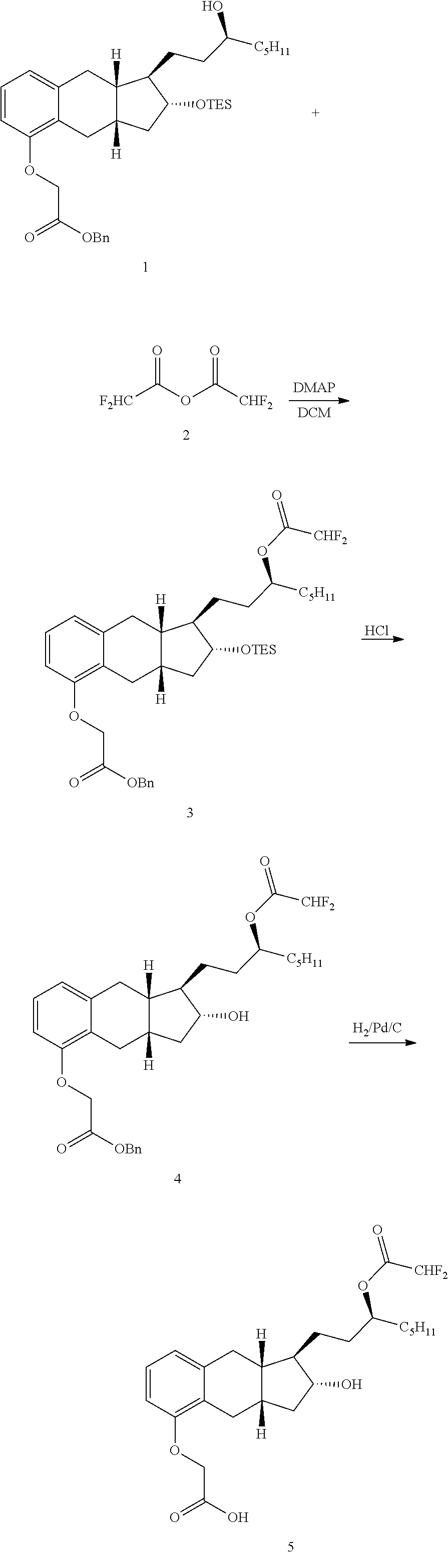

### Experimental

### Synthesis of Mono-TES Treprostinil Benzyl Ester Side Chain Difluoroacetate (3)

To a stirring solution of mono-TES treprostinil benzyl ester (1) (1.01 g, 1.70 mmol) in DCM (20 mL) and DMAP (415 mg, 3.40 mmol) at room temperature was added difluoroacetic anhydride (2) (277 μL, 2.55 mmol). The reaction mixture was stirred at room temperature for 1 h. It was checked by TLC and the reaction was complete. The reaction mixture was concentrated in vacuo to give crude product (1.87 g) which was purified by column chromatography to give mono-TES treprostinil benzyl ester side chain difluoroacetate (3) (0.96 g, 84% yield) (99.19% HPLC purity). The compound 3 was characterized by ¹H NMR and MS.

### Synthesis of Treprostinil Benzyl Ester Side Chain Difluoroacetate (4)

To a stirring solution of mono-TES treprostinil benzyl ester side chain difluoroacetate (3) (0.94 g, 1.40 mmol) in tetrahydrofuran (THF) (20 mL) and water (4 mL) at room temperature was added HCl (2N) (0.7 mL, 1.40 mmol). The reaction mixture was stirred at room temperature for 1 h. It was checked by TLC and the reaction was complete. Water (20 mL) and ethyl acetate (20 mL) were added and layers were separated. The aqueous layer was extracted with ethyl acetate (2×10 mL). The combined organic layers were washed with brine, dried over sodium sulfate. filtered and the filtrate was concentrated in vacuo to give crude product (0.93 g) which was purified by column chromatography to give treprostinil benzyl ester side chain difluoroacetate (4) (0.77 g, 99% yield) (98.92% HPLC purity). The compound 4 was characterized by ¹H NMR and MS.

### Synthesis of Treprostinil Side Chain Difluoroacetate (5)

To a stirring solution of treprostinil benzyl ester side chain difluoroacetate (4) (072 g, 1.29 mmol) in ethyl acetate (15 mL) was added palladium on carbon (5 wt. %, 50% water) (80 mg). The reaction mixture was evacuated under house vacuum, filled with hydrogen (repeat this 2 times), connected to hydrogen balloon and stirred at room temperature for 1 h. It was checked by TLC and the reaction was complete. The mixture was filtered through a Celite pad and washed by ethyl acetate. The filtrate was concentrated in vacuo to produce treprostinil side chain difluoroacetate (5) (0.61 g, 99% yield) (98.83% HPLC purity). Compound 5 was characterized by ¹H NMR, ¹³C NMR, IR and MS.

### 3.11. Synthesis of Treprostinil Di(trifluoroacetate)

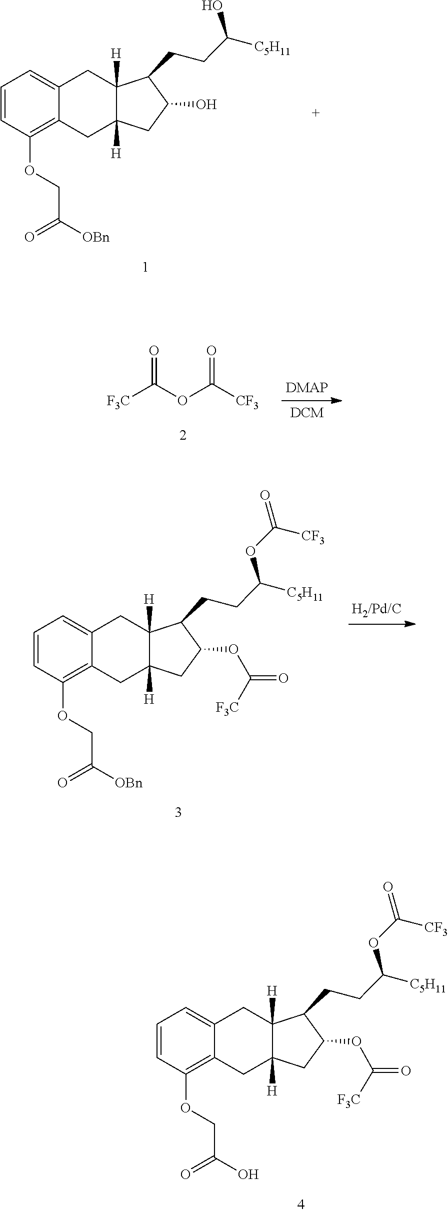

### Experimental

### Synthesis of Treprostinil Benzyl Ester Di(Trifluoroacetate) (3)

To a stirring solution of treprostinil benzyl ester (1) (1.60 g, 3.33 mmol) in DCM (35 mL) and DMAP (2.03 g, 16.55 mmol) at room temperature was added trifluoroacetic anhydride (2) (1.39 mL, 9.99 mmol). The reaction mixture was stirred at room temperature for 3 h. It was checked by TLC and the reaction was complete. The reaction mixture was concentrated in vacuo to give crude product (6.51 g) which was purified by column chromatography to give treprostinil benzyl ester di(trifluoroacetate) (3) (0.79 g, 35% yield) (98.79% HPLC purity). The compound 3 was characterized by ¹H NMR and MS.

### Synthesis of Treprostinil Di(Trifluoroacetate) (4)

To a stirring solution of treprostinil benzyl ester di(trifluoroacetate) (3) (0.75 g, 1.11 mmol) in THF (15 mL) was added palladium on carbon (5 wt. %, 50% water) (75 mg). The reaction mixture was evacuated under house vacuum, filled with hydrogen (repeat this 2 times), connected to hydrogen balloon and stirred at room temperature for 1 h. It was checked by TLC and the reaction was complete. The mixture was filtered through a Celite pad and washed by THF. The filtrate was concentrated in vacuo to produce treprostinil di(trifluoroacetate) (4) (0.40 g, 62% yield) (92.60% HPLC purity). Compound 4 was characterized by ¹H NMR and MS.

### 3.12. Synthesis of Treprostinil Di(diflouroacetate)

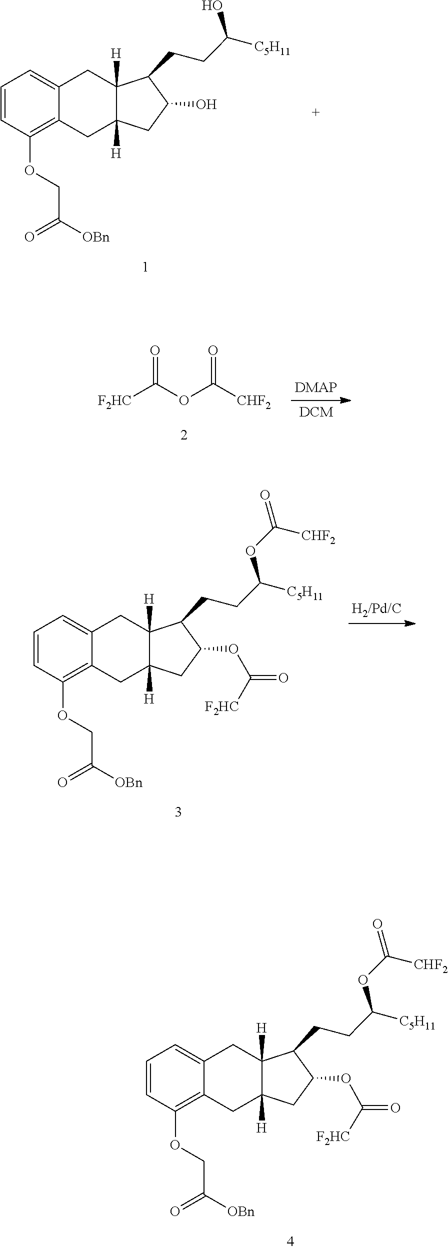

### Experimental

### Synthesis of Treprostinil Benzyl Ester Di(Difluoroacetate) (3)

To a stirring solution of treprostinil benzyl ester (1) (1.62 g, 3.37 mmol) in DCM (35 mL) and DMAP (1.65 g, 13.48 mmol) at room temperature was added difluoroacetic anhydride (2) (807 μL, 7.41 mmol). The reaction mixture was stirred at room temperature for 1 h. It was checked by TLC and the reaction was complete. The reaction mixture was concentrated in vacuo to give crude product (6.51 g) which was purified by column chromatography to give treprostinil benzyl ester di(difluoroacetate) (3) (1.61 g, 75% yield) (98.82% HPLC purity). The compound 3 was characterized by ¹H NMR and MS.

### Synthesis of Treprostinil Di(Difluoroacetate) (4)

To a stirring solution of treprostinil benzyl ester di(difluoroacetate) (3) (0.73 g, 1.14 mmol) in ethyl acetate (15 mL) was added palladium on carbon (5 wt. %, 50% water) (80 mg). The reaction mixture was evacuated under house vacuum, filled with hydrogen (repeat this 2 times), connected to hydrogen balloon and stirred at room temperature for 1 h. It was checked by TLC and the reaction was complete. The reaction mixture was filtered through a Celite pad and washed with ethyl acetate. The filtrate was concentrated in vacuo to produce treprostinil di(difluoroacetate) (4) (0.65 g) as sticky oil. Ethyl acetate (1 mL) was added to make clear solution. This solution was added into hexanes while stirring led to form solid which was filtered. The solid was dried in the air overnight to give product 0.48 g (77% yield) (98.44% HPLC purity) (MP: 38-40° C.). Compound 4 was characterized by ¹H NMR, ¹³C NMR, IR and MS.

### 3.13. Synthesis of Treprostinil Cyclopentyl Diflouroacetate

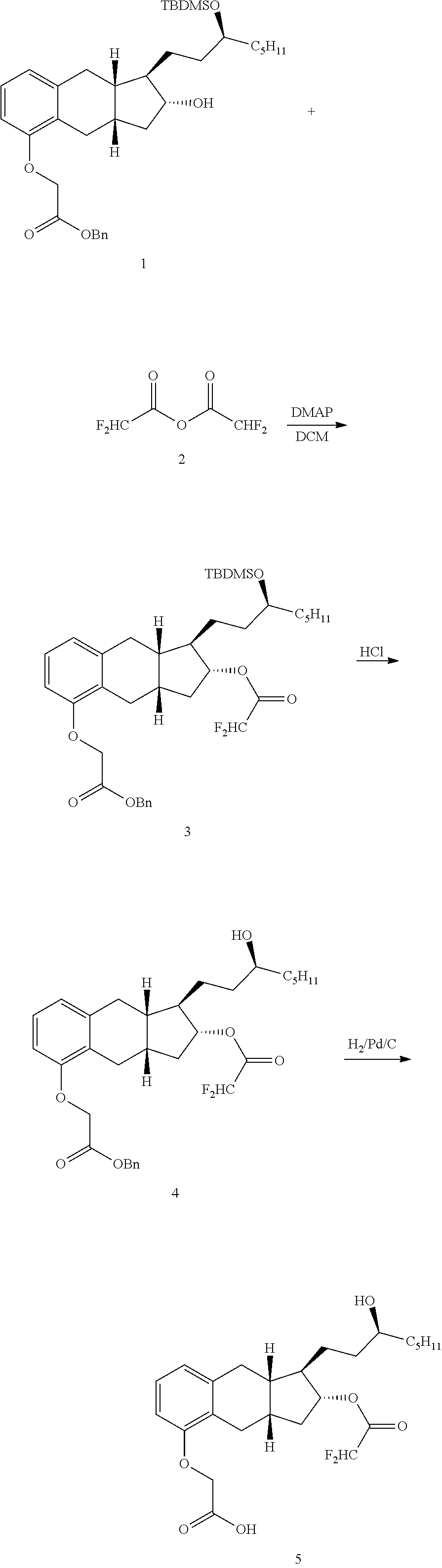

### Experimental

### Synthesis of Treprostinil Benzyl Ester Side Chain TBDMS Cyclopentyl Difluoroacetate (3)

To a stirring solution of treprostinil benzyl ester side chain TBDMS (1) (1.52 g, 2.55 mmol) in DCM (30 mL) and DMAP (623 mg, 5.10 mmol) at room temperature was added difluoroacetic anhydride (2) (420 μL, 3.83 mmol). The reaction mixture was stirred at room temperature for 1 h. It was checked by TLC and the reaction was complete. The reaction mixture was concentrated in vacuo to give crude product (2.12 g) which was purified by column chromatography to give treprostinil benzyl ester side chain TBDMS cyclopentyl difluoroacetate (3) (1.38 g, 81% yield) (99.05% HPLC purity). The compound 3 was characterized by ¹H NMR and MS.

### Synthesis of Treprostinil Benzyl Ester Cyclopentyl Difluoroacetate (4)

To a stirring solution of treprostinil benzyl ester side chain TBDMS cyclopentyl difluoroacetate (3) (1.35 g, 2.01 mmol) in iso-propyl alcohol (IPA) (30 mL) at room temperature was added HCl (2N) (3.0 mL, 6.00 mmol). The reaction mixture was stirred at room temperature for 5 h. It was checked by TLC and reaction was almost complete. Water (20 mL) was added and concentrated in vacuo to distill out most of IPA. EA (20 mL) was added and layers were separated. The aqueous layer was extracted with ethyl acetate (2×20 mL). The combined organic layers were washed with brine and dried over sodium sulfate. It was filtrated and the filtrate was concentrated in vacuo to give crude product (1.19 g) which was purified by column chromatography to give treprostinil benzyl ester cyclopentyl difluoroacetate (4) (0.79 g, 70% yield) (97.65% HPLC purity). The compound 4 was characterized by ¹H NMR and MS.

### Synthesis of Treprostinil Cyclopentyl Difluoroacetate (5)

To a stirring solution of treprostinil benzyl ester cyclopentyl difluoroacetate (4) (0.76 g, 1.37 mmol) in ethyl acetate (15 mL) was added palladium on carbon (5 wt. %, 50% water) (80 mg). The reaction mixture was evacuated under house vacuum, filled with hydrogen (repeat this 2 times), connected to hydrogen balloon and stirred at room temperature for 3 h. It was checked by TLC and the reaction was complete. The mixture was filtered through a Celite pad and washed with ethyl acetate. The filtrate was concentrated in vacuo to produce treprostinil cyclopentyl difluoroacetate (5) (0.62 g, 97% yield) (96.69% HPLC purity). Compound 5 was characterized by ¹H NMR, ¹³C NMR, IR and MS.

### 3.14. Synthesis of Treprostinil Side Chain Diflouroacetate Cyclopentyl Phosphate

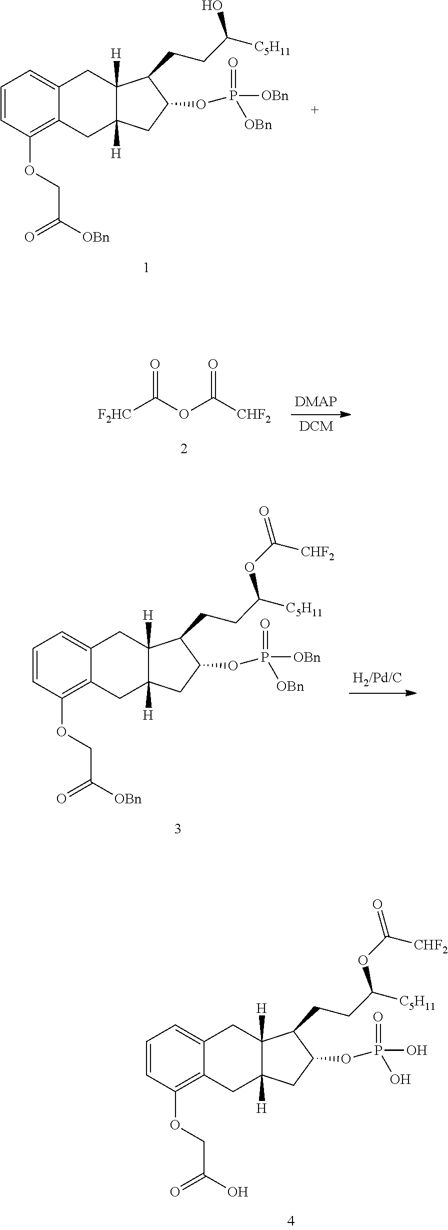

### Experimental

### Synthesis of Treprostinil Benzyl Ester Side Chain Difluoroacetate Cyclopentyl Dibenzylphosphate (3)

To a stirring solution of treprostinil benzyl ester cyclopentyl dibenzylphosphate (1) (1.25 g, 1.68 mmol) in DCM (25 mL) and DMAP (410 mg, 3.36 mmol) at room temperature was added difluoroacetic anhydride (2) (275 μL, 2.52 mmol). The reaction mixture was stirred at room temperature for 1 h. It was checked by TLC and the reaction was complete. The reaction mixture was concentrated in vacuo to give crude product (2.23 g) which was purified by column chromatography to give treprostinil benzyl ester side chain difluoroacetate cyclopentyl dibenzylphosphate (3) (1.25 g, 90% yield) (99.60% HPLC purity). The compound 3 was characterized by ¹H NMR and MS.

### Synthesis of Treprostinil Side Chain Difluoroacetate Cyclopentyl Phosphate (4)

To a stirring solution of treprostinil benzyl ester side chain difluoroacetate cyclopentyl dibenzylphosphate (3) (1.21 g, 1.48 mmol) in tetrahydrofuran (THF) (25 mL) was added palladium on carbon (5 wt. %, 50% water) (350 mg). The reaction mixture was evacuated under house vacuum, filled with hydrogen (repeat this 2 times) and connected to hydrogen balloon and stirred at room temperature for 4 h. It was checked by TLC, ¹H NMR, ³¹P NMR and the reaction was complete by TLC as well as NMR. The mixture was filtered through a Celite pad and washed by THF. The filtrate was concentrated in vacuo to produce treprostinil side chain difluoroacetate cyclopentyl phosphate (4) (0.80 g, 99% yield). This crude compound dissolved in ethyl acetate (2 mL). This solution was added into hexanes (50 mL) while stirring to form solid. It was filtered and the solid was dried in the air to give compound 4 (0.65 g, 80% yield) (97.83% HPLC purity) as white solid (MP: 125-128° C.). Compound 4 was characterized by ¹H NMR, ¹³C NMR, ³¹P NMR, IR and MS.

### 3.15. Synthesis of Treprostinil Side Chain Phosphate Cyclopentyl Diflouroacetate

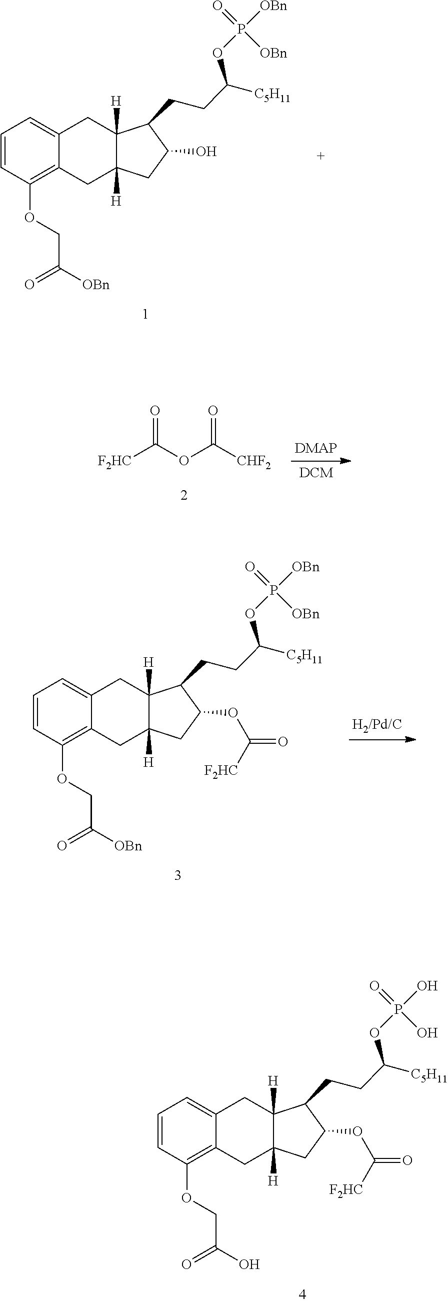

### Experimental

### Synthesis of Treprostinil Benzyl Ester Side Chain Dibenzylphosphate Cyclopentyl Difluoroacetate (3)

To a stirring solution of treprostinil benzyl ester side chain dibenzylphosphate (1) (1.26 g, 1.70 mmol) in DCM (25 mL) and DMAP (415 mg, 3.40 mmol) at room temperature was added difluoroacetic anhydride (2) (222 μL, 2.04 mmol). The reaction mixture was stirred at room temperature for 3 h. It was checked by TLC and the reaction was complete. The reaction mixture was concentrated in vacuo to give crude product (2.34 g) which was purified by column chromatography to give treprostinil benzyl ester side chain dibenzylphosphate cyclopentyl difluoroacetate (3) (1.13 g, 81% yield) (99.60% HPLC purity). The compound 3 was characterized by ¹H NMR and MS.

### Synthesis of Treprostinil Side Chain Phosphate Cyclopentyl Difluoroacetate (4)

To a stirring solution of treprostinil benzyl ester side chain dibenzylphosphate cyclopentyl difluoroacetate (3) (1.11 g, 1.35 mmol) in tetrahydrofuran (THF) (25 mL) was added palladium on carbon (5 wt. %, 50% water) (300 mg). The reaction mixture was evacuated under house vacuum, filled with hydrogen (repeat this 2 times), connected to hydrogen balloon and stirred at room temperature for 3 h. It was checked by TLC, ¹H NMR, ³¹P NMR and the reaction was complete. The mixture was filtered through a Celite pad and washed by THF. The filtrate was concentrated in vacuo to produce treprostinil side chain phosphate cyclopentyl difluoroacetate (4). The compound 4 was characterized by ¹H NMR, ¹³C NMR, ³¹P NMR, IR and MS.

### 3.16. Synthesis of Treprostinil Side Chain Palmitate

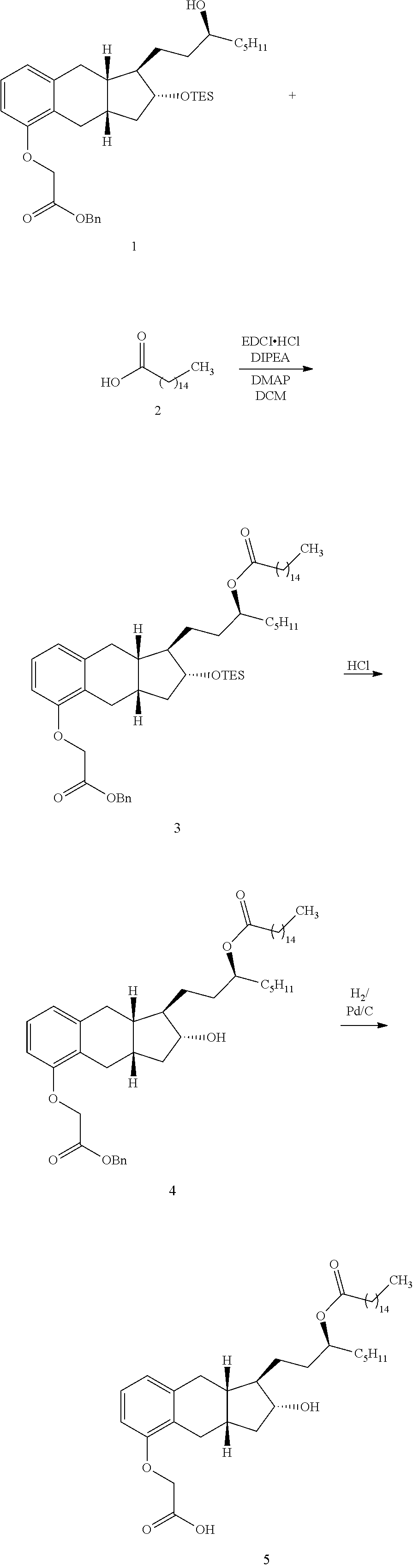

### Experimental

### Synthesis of Mono-TES Treprostinil Benzyl Ester Side Chain Palmitate (3)

To a stirring solution of mono-TES treprostinil benzyl ester (1) (3.72 g, 6.25 mmol), palmitic acid (2) (1.92 g, 7.50 mmol), DIPEA (2.72 mL, 15.63 mmol) and DMAP (153 mg, 1.25 mmol) in dichloromethane (DCM) (70 mL) at room temperature under argon was added EDCl·HCl (3.00 g, 15.63 mmol). The reaction mixture was stirred at room temperature overnight. It was checked by TLC and the reaction was almost complete. Water (20 mL) was added and layers were separated. The aqueous layer was extracted with DCM (2×10 mL). The combined DCM layers were washed with brine, dried over sodium sulfate, filtered, concentrated the filtrate in vacuo to give crude product (6.90 g) which was purified by column chromatography to give mono-TES treprostinil benzyl ester side chain palmitate (3) (4.58 g, 88% yield). The compound (3) was characterized by ¹H NMR and MS.

### Synthesis of Treprostinil Benzyl Ester Side Chain Palmitate (4)

To a stirring solution of mono-TES treprostinil benzyl ester side chain palmitate (3) (1.98 g, 2.37 mmol) in THF (50 mL) and water (10 mL) at room temperature was added HCl (2N) (1.2 mL, 2.40 mmol). The reaction mixture was stirred at room temperature for 1 h. It was checked by TLC and the reaction was complete. Water (50 mL) and ethyl acetate (50 mL) were added and the layers were separated. The aqueous layer was extracted with ethyl acetate (2×20 mL). The combined organic layers were washed with brine, dried over sodium sulfate. It was filtered and the filtrate was concentrated in vacuo to give crude product (2.23 g) which was purified by column chromatography to give treprostinil benzyl ester side chain palmitate (4) (1.72 g, 99% yield) (99.89% HPLC purity). The compound 4 was characterized by ¹H NMR and MS.

### Synthesis of Treprostinil Side Chain Palmitate (5)

To a stirring solution of treprostinil benzyl ester side chain palmitate (4) (1.58 g, 2.20 mmol) in ethyl acetate (30 mL) was added palladium on carbon (5 wt. %, 50% water) (150 mg). The reaction mixture was evacuated under house vacuum, filled with hydrogen (repeat this 2 times), connected to hydrogen balloon, stirred at room temperature for 2 h. It was checked by TLC and the reaction was complete. The mixture was filtered through a Celite pad and washed with ethyl acetate. The filtrate was concentrated in vacuo to produce treprostinil side chain palmitate (5) (1.38 g, 99% yield) (99.38% HPLC purity) (MP: 46-48° C.). Compound 5 was characterized by ¹H NMR, ¹³C NMR, IR and MS.

### 3.17. Synthesis of Treprostinil Cyclopentyl Palmitate

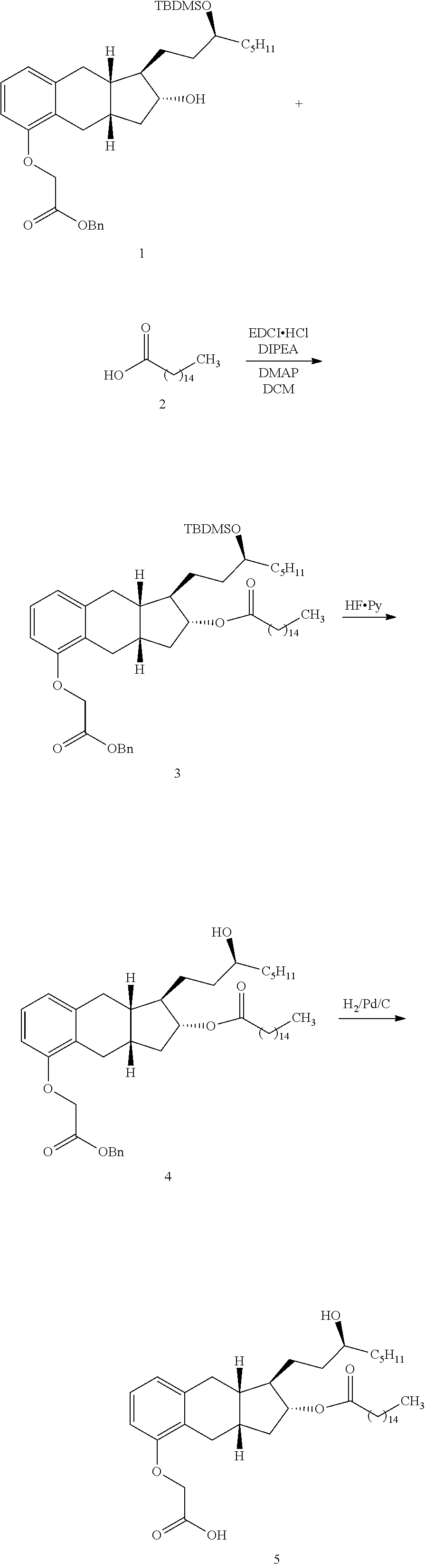

### Experimental

### Synthesis of Treprostinil Benzyl Ester Side Chain TBDMS Cyclopentyl Palmitate (3)

To a stirring solution of treprostinil benzyl ester side chain TBDMS (1) (1.61 g, 2.70 mmol), palmitic acid (2) (831 mg, 3.24 mmol), DIPEA (1.2 mL, 6.75 mmol) and DMAP (66 mg, 0.54 mmol) in dichloromethane (DCM) (40 mL) at room temperature under argon was added EDCl·HCl (1.29 g, 6.75 mmol). The reaction mixture was stirred at room temperature overnight and checked by TLC. Water (20 mL) was added, and the layers were separated. The aqueous layer was extracted with DCM (2×10 mL). The combined DCM layers were washed with brine, dried over sodium sulfate and filtered. The filtrate was concentrated in vacuo to give crude product (3.08 g) which was purified by column chromatography to give treprostinil benzyl ester side chain TBDMS cyclopentyl palmitate (3) (2.13 g, 95% yield) (87.03% HPLC purity). The compound 3 was characterized by ¹H NMR and MS.

### Synthesis of Treprostinil Benzyl Ester Cyclopentyl Palmitate (4)

To a stirring solution of treprostinil benzyl ester side chain TBDMS cyclopentyl palmitate (3) (2.00 g, 2.40 mmol) in THF (20 mL) in a plastic tube cooled to 0° C. was added HF·Py (4.3 mL, 36 mmol). The reaction mixture was stirred at that temperature and slowly warm to room temperature for 5 h. It was checked by TLC and reaction was complete. Reaction was quenched with saturated aq. sodium bicarbonate solution until pH-7. The reaction mixture was extracted with ethyl acetate (3×20 mL). The combined organic layers were washed with brine, dried over sodium sulfate. It was filtrated and the filtrate was concentrated in vacuo to give crude product (1.61 g) which was purified by column chromatography to give treprostinil benzyl ester cyclopentyl palmitate (4) (1.29 g, 75% yield) (98.72% HPLC purity). The compound (4) was characterized by ¹H NMR and MS.

### Synthesis of Treprostinil Cyclopentyl Palmitate (5)

To a stirring solution of treprostinil benzyl ester cyclopentyl palmitate (4) (1.19 g, 1.65 mmol) in ethyl acetate (25 mL) was added palladium on carbon (5 wt. %, 50% water) (120 mg). The reaction mixture was evacuated under house vacuum, filled with hydrogen (repeat this 2 times), connected to hydrogen balloon and stirred at room temperature for 2 h. It was checked by TLC and the reaction was found to be complete. The mixture was filtered through a Celite pad and washed with ethyl acetate. The filtrate was concentrated in vacuo to produce treprostinil cyclopentyl palmitate (5) (1.00 g, 99% yield) (97.65% HPLC purity) (MP: 74-76° C.). Compound 5 was characterized by ¹H NMR, ¹³C NMR, IR and MS.

### 3.18. Synthesis of Treprostinil Hexadecyl Ester

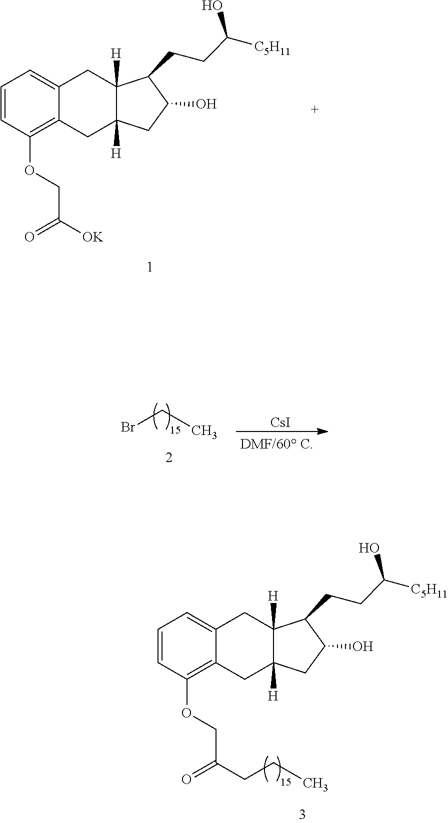

### Experimental

### Synthesis of Treprostinil Hexadecyl Ester (3)

To a stirring solution of treprostinil potassium salt (1) (0.50 g, 1.17 mmol) in DMF (10 mL) at room temperature were added 1-bromohexadecane (2) (715 μL, 2.34 mmol) and cesium iodide (330 mg, 1.29 mmol). The reaction mixture was stirred at 60° C. oil bath for 5 h. It was checked by TLC and the reaction was complete. Saturated aq. ammonium chloride (20 mL) was added, followed by ethyl acetate (20 mL). The aqueous layer was extracted with ethyl acetate (2×20 mL). The combined organic layers were washed with brine, dried over sodium sulfate. It was filtered and the filtrate was concentrated in vacuo to give crude product (1.19 g) which was purified by column chromatography to give treprostinil hexadecyl ester (3) (0.73 g, 99% yield) (99.70% HPLC purity) (MP: 51-53° C.). The compound 3 was characterized by ¹H NMR ¹³C NMR, IR and MS.

### 3.19. Synthesis of Treprostinil Glycolphosphate

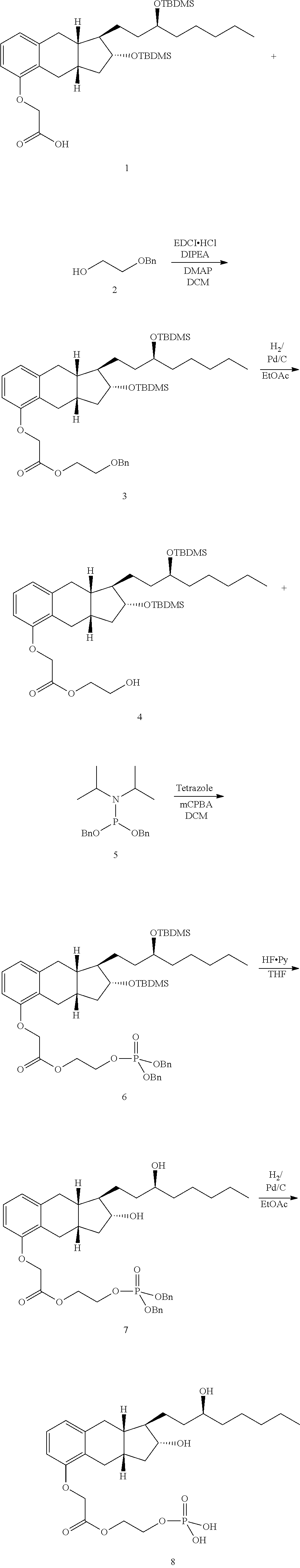

### Experimental

### Synthesis of Di-TBDMS treprostinil benzylglycol ester (3)

To a stirring solution of Di-TBDMS treprostinil (1) (5.13 g, 8.29 mmol), benzyl glycol (2) (1.3 mL, 9.12 mmol), DIPEA (3.6 mL, 20.73 mmol) and DMAP (2.53 g, 20.73 mmol) in dichloromethane (DCM) (50 mL) at room temperature under argon was added EDCl·HCl (3.97 g, 20.73 mmol). The reaction mixture stirred at room temperature for 2 h and checked by TLC. Water (50 mL) was added and layers were separated. Aqueous layer was extracted with DCM (2×20 mL). Combined organic layers were washed with brine, dried over sodium sulfate. It was filtered and the filtrated was concentrated in vacuo to give crude product (8.87 g), which was purified by column chromatography to give Di-TBDMS treprostinil benzylglycol ester (3) (5.13 g, 82% yield) (97.08% HPLC purity). The compound (3) was characterized by ¹H NMR and MS.

### Synthesis of Di-TBDMS treprostinil glycol ester (4)

To a stirring solution of Di-TBDMS treprostinil benzylglycol ester (3) (5.05 g, 6.70 mmol) in ethyl acetate (80 mL) was added palladium on carbon (0.50 g, 5 wt %, 50% water). The system was evacuated and replaced with hydrogen (from hydrogen balloon) (repeated 2 times). The system was connect to hydrogen balloon, stirred for 6 h at room temperature and checked by TLC. It was filtered through Celite pad and the filtrated was concentrated in vacuo to give crude product (4.93 g). It was purified by column chromatography to give Di-TBDMS treprostinil glycol ester (4) (4.07 g, 92% yield) (98.90% HPLC purity). Compound 4 was characterized by ¹H and MS.

### Synthesis of Di-TBDMS treprostinil glycol dibenzylphosphate (6)

To a stirring solution of Di-TBDMS treprostinil glycol ester (4) (1.08 g, 1.63 mmol), tetrazole (10.9 mL, 0.45 M in acetonitrile, 4.89 mmol) in DCM (40 mL) was added dibenzyl-N,N-diisopropylphosphoramidite (5) (1.13 g, 3.26 mmol) at room temperature under argon. It was stirred for 3 h and checked by TLC and the reaction was complete at this stage. The system was cooled to −78° C. (dry ice-acetone) and mCPBA (70-75%) (0.87 g, 5.05 mmol) was added. The reaction mixture was stirred for 3 h, checked by TLC, and reaction was complete. Sodium sulfite solution (10%) was added and stirred overnight. Layers were separated and the DCM layer was checked with Peroxide 100 Tip to make sure no peroxide exist. The DCM layer was washed by sodium bicarbonate solution (sat.), water, brine and dried over sodium sulfate. It was filtered and the filtrate was concentrated in vacuo to give crude product (2.33 g), which was purified by column chromatography to give Di-TBDMS treprostinil glycol dibenzylphosphate (6) (1.42 g, 95% yield) (86.27% HPLC purity). The compound 6 was characterized by ¹H NMR and MS.

### Synthesis of treprostinil glycol dibenzylphosphate (7)

To a stirring solution of Di-TBDMS treprostinil glycol dibenzylphosphate (6) (1.08 g, 1.17 mmol) in THF in a teflon tube was added hydrogen fluoride pyridine solution in THF (2.5 mL). The mixture was stirred at room temperature for 4 h and checked by TLC and quenched by sodium bicarbonate slowly to pH-8. It was extracted with EtOAc (50 mL, 2×20 mL) and the combined organic layers washed with brine, dried over sodium sulfate. It was filtered and the filtrate was concentrated in vacuo to give crude product (1.70 g) which was purified by column chromatography to give treprostinil glycol dibenzylphosphate (7) (0.63 g, 77% yield) (99.04% HPLC purity). The compound 7 was characterized by ¹H NMR and MS.

### Synthesis of treprostinil glycolphosphate (8)

To a stirring solution of treprostinil glycol dibenzylphosphate (7) (0.21 g, 0.30 mmol) in ethyl acetate (10 mL) was added palladium on carbon (50 mg, 5 wt %, 50% water). The system was evacuated and replaced with hydrogen from hydrogen balloon (repeat 2 more times). Then the system was connected to hydrogen balloon and stirred at room temperature for 2 h and checked by TLC, the reaction was complete. It was filtered through a Celite pad and washed with EtOAc (2×10 mL). The filtrate was concentrated in vacuo to give treprostinil glycolphosphate 8 (0.12 g, 78% yield) (99.13% HPLC purity). Compound 8 was characterized by ¹H NMR, ¹³C NMR, ³¹P NMR, IR and MS.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A compound of formula (1), an enantiomer thereof or a pharmaceutically acceptable salt thereof: 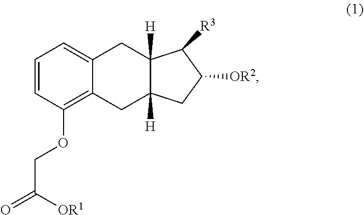
wherein R¹ is H, a C₁-C₃ alkyl group or a carboxylic acid protecting group; R² is H or an alcohol protecting group; and R³ is 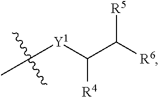
wherein Y¹ is —C≡C—; —CH═CH—; or —(CH₂)ₘ—, m is an integer from 0 to 5; R⁴ is H, OH or ═O; R⁵ is H, OH, a substituted or unsubstituted alkyl group, a substituted or unsubstituted aryl, a substituted or unsubstituted heterocyclic group or a substituted or unsubstituted carbocyclic group; R⁶ is a substituted or unsubstituted alkyl group, a substituted or unsubstituted alkenyl group, a substituted or unsubstituted alkynyl group, a substituted or unsubstituted aryl group, a substituted or unsubstituted cycloalkyl group, wherein for R¹ and R² each being H, R³ is not

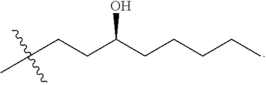 [SMILES: *CC[C@@H](O)CCCCC]

2. The compound of claim 1, wherein R¹ is H or CH₃.

3. The compound of claim 1, wherein R² is H.

4. The compound of claim 1, wherein R³ is 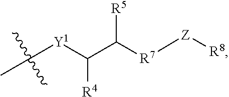
wherein R⁷ is an alkyl group, an alkenyl group or an alkynyl group; Z is O, CH₂, NH or S and R⁸ is a heterocyclic group.

5. The compound of claim 1, wherein R³ is 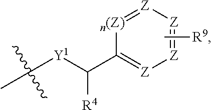
wherein each Z is independently selected from CH, N, O or S, n is 0 or 1, R⁹ is an alkyl group, an aryl group, an electron withdrawing group, an electron donating group, a heterocycle or a carbocycle.

6. The compound of claim 1, wherein R³ is 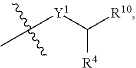
wherein R¹⁰ is a cycloalkyl group having from 3 to 8 carbon atoms, wherein one or more carbon atoms in the cycloalkyl group may be optionally replaced with a heteroatom selected from O, N and S.

7. The compound of claim 1, wherein R³ is selected from

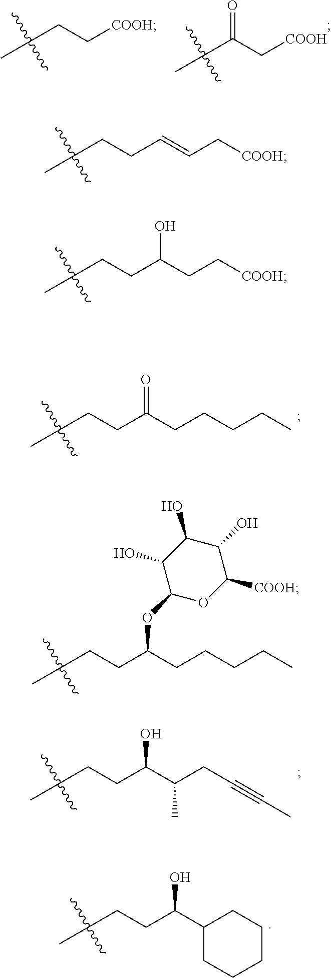

8. A compound having formula (2), an enantiomer thereof or a pharmaceutically acceptable salt thereof: 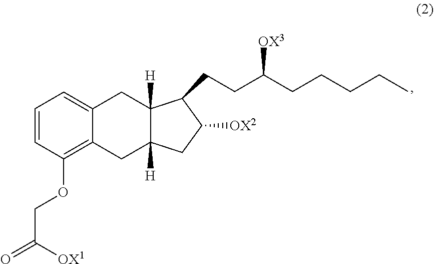
wherein X¹ is hydrogen, 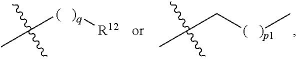
wherein q is 1, 2 or 3, p1 is an integer from 1 to 20, R¹² is a phosphate group or COOH; each of X² and X³ is independently hydrogen, 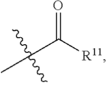
a phosphate, or 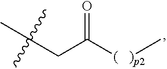
wherein p2 is an integer from 1 to 20, R¹¹ is a C₁-C₈ alkyl group, one or more carbon atoms of said C₁-C₈ alkyl group may be optionally replaced with O, one or more hydrogen atoms of said C₁-C₈ alkyl group may be optionally replaced with halogen; X² is hydrogen, wherein X¹, X² and X³ are not all hydrogen; for X¹ being hydrogen and one of X² and X³ being a phosphate, the other of X² and X³ is not a phosphate or hydrogen; for X¹ being hydrogen and one of X² and X³ being 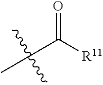
with R¹¹ being an unsubstituted C₁-C₈ alkyl group, the other of X² and X³ is not hydrogen and X² or X³ are not the same.

9. The compound of claim 8, wherein X¹ is

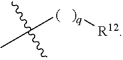

10. The compound of claim 9, wherein X² and X³ are the same.

11. The compound of claim 8, wherein each of X² and X³ is hydrogen.

12. The compound of claim 9, wherein each of X² and X³ is

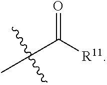

13. The compound of claim 8, wherein X¹ is hydrogen.

14. The compound of claim 13, wherein one of X² and X³ is a phosphate and the other of X² and X³ is

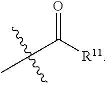

15. The compound of claim 13, wherein at least one of X² and X³ is 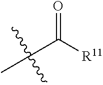
with R¹¹ being a C₁-C₈ alkyl group having one or more carbon atoms of said C₁-C₈ alkyl group replaced with O or having one or more hydrogen atoms replaced with halogen.

16. The compound of claim 15, wherein at least one of X² and X³ is 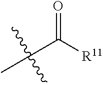
with R¹¹ being a C₁-C₈ alkyl group having one or more hydrogen atoms replaced with halogen.

17. The compound of claim 16, wherein each of X² and X³ is 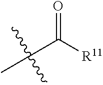
with R¹¹ being a C₁-C₈ alkyl group having one or more hydrogen atoms replaced with halogen.

18. The compound of claim 17, wherein X² and X³ are the same.

19. The compound of claim 8, wherein at least one of X² and X³ being 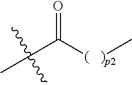
with p2 being an integer from 12 to 16 or X¹ being 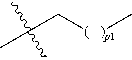
with p1 being an integer from 12 to 16.

20. The compound of claim 8, wherein (a) each of X² and X³ is hydrogen and X¹ is 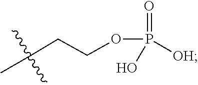 [SMILES: *CCOP(=O)(O)O]
(b) X³ is 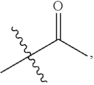 [SMILES: *C(C)=O]
X² is 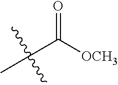 [SMILES: *C(=O)OC]
and X¹ is hydrogen; (c) X³ is 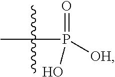 [SMILES: *P(=O)(O)O]
X² is 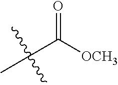 [SMILES: *C(=O)OC]
and X¹ is hydrogen; (d) X³ is 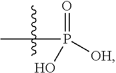 [SMILES: *P(=O)(O)O]
X² is 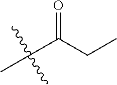 [SMILES: *C(=O)CC]
and X¹ is hydrogen; (e) each of X² and X³ is 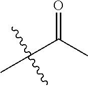 [SMILES: *C(C)=O]
and X¹ is 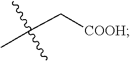 [SMILES: *CC(=O)O]
(f) each of X² and X³ is 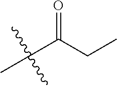 [SMILES: *C(=O)CC]
and X¹ is 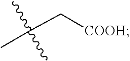 [SMILES: *CC(=O)O]
(g) each X¹ and X² is H and X³ is 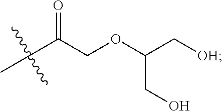 [SMILES: *C(=O)COC(CO)CO]
(h) X³ is 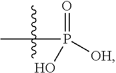 [SMILES: *P(=O)(O)O]
X² is 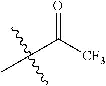 [SMILES: *C(=O)C(F)(F)F]
and X¹ is hydrogen; (i) X² is 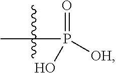 [SMILES: *P(=O)(O)O]
X³ is 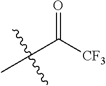 [SMILES: *C(=O)C(F)(F)F]
and X¹ is hydrogen; (j) each of X¹ and X² is hydrogen and X³ is 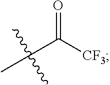 [SMILES: *C(=O)C(F)(F)F]
(k) each of X¹ and X² is hydrogen and X³ is 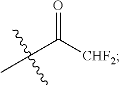 [SMILES: *C(=O)C(F)F]
(l) each of X² and X³ is 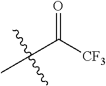 [SMILES: *C(=O)C(F)(F)F]
and X¹ is hydrogen; (m) each of X² and X³ is hydrogen and X³ is 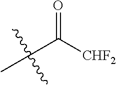 [SMILES: *C(=O)C(F)F]
and X¹ is hydrogen; (n) each of X¹ and X³ is hydrogen and X³ is 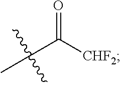 [SMILES: *C(=O)C(F)F]
(o) X¹ is hydrogen; X² is 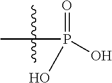 [SMILES: *P(=O)(O)O]
and X³ is 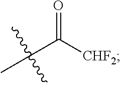 [SMILES: *C(=O)C(F)F]
(p) X¹ is hydrogen; X² is 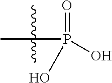 [SMILES: *P(=O)(O)O]
and X³ is 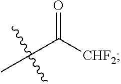 [SMILES: *C(=O)C(F)F]
(q) each of X² and X³ is hydrogen and X¹ is 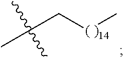
(r) each of X¹ and X² is hydrogen and X³ is 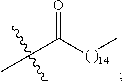
(s) each of X¹ and X³ is hydrogen and X² is 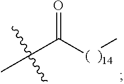
or (t) X¹ is 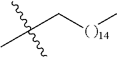
and each X² and X³ is

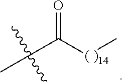

21. A pharmaceutical composition comprising the compound of claim 1 and a pharmaceutically acceptable carrier.

22. A method of treating pulmonary hypertension comprising administering to a subject in need thereof an effective amount of the compound of claim 1.

23. A compound of formula (3): 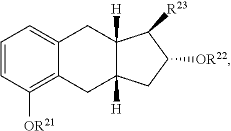
wherein R²¹ is a phenolic protecting group or CH₂COOR²⁴; R²² is an alcohol protecting group; R²³ is a hydroxy terminated alkyl, —C═CH₂, —C≡CH or 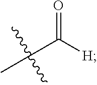
R²⁴ is a carboxylic acid protecting group.

24. The compound of claim 23, wherein R²³ is a hydroxy terminated alkyl.

25. The compound of claim 23 having formula (31):

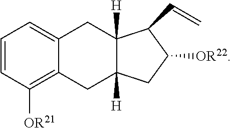

26. The compound of claim 23 having formula (32):

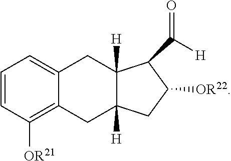

27. The compound of claim 23 having formula (33):

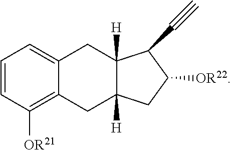

28. The compound of claim 23, wherein R²¹ is C₁-C₄ alkyl, a substituted or unsubstituted benzyl or CH₂COOR²⁴, wherein R²⁴ is C₁-C₄ alkyl or a substituted or unsubstituted benzyl.

29. The compound of claim 23, wherein R²² is an acetyl group or a silyl containing group.
